SEQUENCE LISTING (82 sequences)

SEQ ID NO: 1-2 - skipped (2 entries)

SEQ ID NO: 3 - protein, 9 aa, synthetic construct
  REGION 1..9: note = Peroxisome targeting sequence
LGRGRRSKL

SEQ ID NO: 4 - skipped

SEQ ID NO: 5 - protein, 8 aa, synthetic construct
  REGION 1..8: note = tag
DYKDDDDK

SEQ ID NO: 6 - protein, 6 aa, synthetic construct
  REGION 1..6: note = tag
HHHHHH

SEQ ID NO: 7 - protein, 26 aa, synthetic construct
  REGION 1..26: note = calmodulin tag
KRRWKKNFIAVSAANRFKKISSSGAL

SEQ ID NO: 8 - protein, 9 aa, synthetic construct
  REGION 1..9: note = HA tag
YPYDVPDYA

SEQ ID NO: 9 - protein, 10 aa, synthetic construct
  REGION 1..10: note = Myc tag
EQKLISEEDL

SEQ ID NO: 10 - protein, 38 aa, synthetic construct
  REGION 1..38: note = SBP tag
MDEKTTGWRGGHVVEGLAGELEQLRARLEHHPQGQREP

SEQ ID NO: 11 - protein, 8 aa, synthetic construct
  REGION 1..8: note = Strp tag
WSHPQFEK

SEQ ID NO: 12 - DNA, 27 bp, synthetic construct
  misc_feature 1..27: note = ePTS1 tag
  source 1..27: mol_type = other DNA
ttgggaagaggtagaagatccaaattg

SEQ ID NO: 13 - protein, 238 aa, synthetic construct
  REGION 1..238: note = GFP tag
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 14 - DNA, 714 bp, synthetic construct
  misc_feature 1..714: note = GFP tag
  source 1..714: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaa

SEQ ID NO: 15 - DNA, 4392 bp, synthetic construct
  misc_feature 1..4392: note = Btau COL1A1
  source 1..4392: mol_type = other DNA
atgttcagctttgtggacctccggctcctgctcctcttagcggccaccgccctcctgacgcacggccaagaggagggccaggaagaaggccaagaagaagacatcccaccagtcacctgcgtacagaacggcctcaggtaccatgaccgagacgtgtggaaacccgtgccctgccagatctgtgtctgcgacaacggcaacgtgctgtgcgatgacgtgatctgcgacgaacttaaggactgtcctaacgccaaagtccccacggacgaatgctgccccgtctgccccgaaggccaggaatcacccacggaccaagaaaccaccggagtcgagggaccgaaaggagacactggcccccgaggcccaaggggacccgccggcccccccggccgagatggcatccctggacaacctggacttcccggaccccctggaccccccggacctcccggaccccctggcctcggaggaaactttgctccccagttgtcttacggctatgatgagaaatcaacaggaatttccgtgcctggtcccatgggtccttctggtcctcgtggtctccctggcccccctggcgcacctggtccccaaggtttccaaggcccccctggtgagcctggcgagccaggagcctcaggtcccatgggtccccgtggtccccctggcccccctggcaagaacggagatgatggcgaagctggaaagcctggtcgtcctggtgagcgcgggcctcccggacctcagggtgctcggggattgcctggaacagctggcctccctggaatgaagggacacagaggtttcagtggtttggatggtgccaagggagatgctggtcctgctggccccaagggcgagcctggtagccccggtgaaaatggagctcctggtcagatgggcccccgtggtctgcctggtgagagaggtcgccctggagcccctggccctgctggtgctcgaggaaatgatggtgcgactggtgctgctgggccccctggtcccactggccccgctggtcctcctggtttccctggtgctgtgggtgctaagggtgaaggtggtccccaaggaccccgaggttctgaaggtccccagggtgtacgtggtgagcctggcccccctggccctgctggtgctgctggccctgctggcaaccctggtgctgatggacagcctggtgctaaaggagccaatggcgctcctggtattgctggtgctcctggcttccctggtgcccgaggcccctctggaccccagggccccagcggcccccctggccccaagggtaacagcggtgaacctggtgctcctggcagcaaaggagacactggcgccaagggagaacccggtcccactggtattcaaggcccccctggccccgctggggaagaaggaaagcgaggagcccgaggtgaacctggacctgctggcctgcctggaccccctggcgagcgtggtggacctggaagccgtggtttccctggcgccgacggtgttgctggtcccaagggtcctgctggtgaacgcggtgctcctggccctgctggccccaaaggttctcctggtgaagctggtcgccccggtgaagctggtctgcccggtgccaagggtctgactggaagccctggcagcccgggtcctgatggcaaaactggcccccctggtcccgccggtcaagatggccgccctggacctccaggccctcccggtgcccgtggtcaggctggcgtgatgggtttccctggacctaaaggtgctgctggagagcctggaaaagctggagagcgaggtgttcctggaccccctggcgctgttggtcctgctggcaaagacggagaagctggagctcagggacccccaggacctgctggccccgctggtgagagaggcgaacaaggccctgctggctcccctggattccagggtctccccggccctgctggtcctcctggtgaagcaggcaaacctggtgaacagggtgttcctggagatcttggtgcccccggcccctctggagcaagaggcgagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccggacccaaaggtgctcgtggcagcgctggtccccctggtgctactggtttcccaggtgctgctggccgagtcggtccccccggcccctctggaaatgctggaccccctggccctcctggccctgctggcaaagaaggcagcaaaggcccccgcggtgagactggccccgctgggcgtcccggtgaagtcggtccccctggtccccctggccccgctggtgagaaaggagcccctggtgctgacggacctgctggagctcctggcactcctggacctcaaggtattgctggacagcgtggtgtggtcggcctgcctggtcagagaggagaaagaggcttccctggtcttcctggcccctctggtgaacccggcaaacaaggtccttctggagcaagtggtgaacgtggcccccctggtcccatgggcccccctggattggctggaccccctggcgagtctggacgtgagggagctcctggtgctgaaggatcccctggacgagatggttctcctggcgccaagggtgaccgtggtgagaccggccctgctggacctcctggtgctcctggcgctcccggtgcccccggccctgtcggacctgccggcaagagcggtgatcgtggtgagaccggtcctgctggtcctgctggtcccattggccccgttggtgcccgtggccccgctggaccccaaggcccccgtggtgacaagggtgagacaggcgaacagggcgacagaggcattaagggtcaccgtggcttctctggtctccagggtccccccggccctcccggctctcctggtgagcaaggtccttccggagcctctggtcctgctggtccccgcggtccccctggctctgctggttctcccggcaaagatggactcaatggtctcccaggccccatcggtccccctgggcctcgaggtcgcactggtgatgctggtcctgctggtcctcccggccctcctggaccccctggtcccccaggtcctcccagcggcggctacgacttgagcttcctgccccagccacctcaagagaaggctcacgatggtggccgctactaccgggctgatgatgccaatgtggtccgtgaccgtgacctcgaggtggacaccaccctcaagagcctgagccagcagatcgagaacatccggagccctgaaggcagccgcaagaaccccgcccgcacctgccgtgacctcaagatgtgccactctgactggaagagcggagaatactggattgaccccaaccaaggctgcaacctggatgccattaaggtcttctgcaacatggaaaccggtgagacctgtgtataccccactcagcccagcgtggcccagaagaactggtatatcagcaagaaccccaaggaaaagaggcacgtctggtacggcgagagcatgaccggcggattccagttcgagtatggcggccaggggtccgatcctgccgatgtggccatccagctgactttcctgcgcctgatgtccaccgaggcctcccagaacatcacctaccactgcaagaacagcgtggcctacatggaccagcagactggcaacctcaagaaggccctgctcctccagggctccaacgagatcgagatccgggccgagggcaacagccgcttcacctacagcgtcacctacgatggctgcacgagtcacaccggagcctggggcaagacagtgatcgaatacaaaaccaccaagacctcccgcttgcccatcatcgatgtggcccccttggacgttggcgccccagaccaggaattcggcttcgacgttggccctgcctgcttcctgtaa

SEQ ID NO: 16 - protein, 1463 aa, synthetic construct
  REGION 1..1463: note = Btau COL1A1
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 17 - DNA, 4095 bp, synthetic construct
  misc_feature 1..4095: note = Btau COL1A1
  source 1..4095: mol_type = other DNA
atgctcagctttgtggatacgcggactttgttgctgcttgcagtaacttcgtgcctagcaacatgccaatccttacaagaggcaactgcaagaaagggcccaagtggagatagaggaccacgcggagaaaggggtccaccaggcccaccaggcagagatggtgatgacggcatcccaggccctcctggcccccctggccctcctggcccccctggtcttggcgggaactttgctgctcagtttgatgcaaaaggaggtggccctggaccaatggggctgatgggacctcgcggccctcctggggcttctggagcccctggccctcaaggtttccagggacctccgggtgagcctggtgaacctggtcagactggtcctgcaggtgctcgtggcccgcctggccctcctggcaaggctggtgaggatggtcaccctggaaaacctggacgacctggtgagagaggggttgttggaccacagggtgctcgtggctttcctggaactcctggactccctggcttcaagggcattaggggtcacaatggtctggatggattgaagggacagcctggtgctccaggtgtgaagggtgaacctggtgcccctggtgaaaatggaactccaggtcaaacgggagcccgtggtcttcctggtgagagaggacgtgttggtgcccctggcccagctggtgcccgtggaagtgatggaagtgtgggtcctgtgggccctgctggtcccattgggtctgctggccctccaggcttcccaggtgctcctggccccaagggtgaactcggacctgttggtaaccctggccctgctggtcccgcgggtccccgtggtgaagtgggtctcccaggcctttctggccctgtcggacctcctggaaaccccggagccaatgggcttcctggcgctaagggtgctgctggccttcccggtgttgctggggctcccggcctccctggaccccggggtattcctggccctgttggcgctgctggtgctactggcgccagaggacttgttggtgagcccggcccagctggttcgaaaggagagagcggcaacaagggcgagcctggtgctgttgggcagccaggtcctcctggccccagtggtgaagaaggaaagagaggctccactggagaaatcggacccgctggccccccaggacctcctgggctgaggggaaatcctggctcccgtggtctacctggagctgacggcagagctggtgtcatgggtcctgctggtagccgtggtgcaactggccctgctggtgtgcgaggtcccaatggagattctggtcgccctggagagcctggcctcatgggaccccgaggtttcccaggttcccctggaaatatcggcccagctggtaaagaaggtcctgtgggtctccctggtattgacggcagacctgggcccattggcccagcgggagcaagaggagagcctggcaacattggattccctggacccaaaggccccagtggtgatcctggcaaagctggtgaaaaaggtcatgctggtcttgctggtgctcggggcgctccaggtcccgatggcaacaacggtgctcagggaccccctggactacagggtgtccaaggtggaaaaggtgaacagggtcctgctggtcctccaggcttccagggtctgcctggccctgcaggcacagctggtgaagctggcaaaccaggagaaaggggtatccctggtgaatttggtctccctggccctgctggtgcaagaggggagcgggggcccccaggtgaaagtggtgctgctgggcctactgggcctattggaagccgaggtccttctggacccccagggcctgatggaaacaagggtgaaccgggtgtggttggcgctccaggcactgctggcccatctggtcctagcggactcccaggagagaggggtgcggctggcattcctggaggcaagggagaaaagggtgaaactggtctcagaggtgacattggtagccctggtagagatggtgctcgtggtgctcctggtgctattggtgctcctggccctgctggagccaatggggaccggggtgaagctggtcccgctggccctgctggccctgctggtcctcgtggtagccctggtgaacgtggtgaggtcggtcccgctggccccaacggatttgctggtcctgctggtgctgctggtcaacctggtgctaaaggagagagaggaaccaaaggacccaagggtgaaaatggtcctgttggtcccacaggccccgttggagctgccggtccgtctggtccaaatggcccacctggtcctgctggaagtcgtggtgatggagggccccctggggctactggtttccctggtgctgctggacggactggtccccctggaccctctggtatctctggcccccctggcccccctggtcctgctggtaaagaagggcttcgtgggcctcgtggtgaccaaggtccagttggtcgaagtggagagacaggtgcctctggccctcctggctttgttggtgagaagggtccctctggagagcctggtactgctgggcctcctggaaccccaggtccacaaggccttcttggtgctcctggttttctgggtctcccaggctctagaggtgagcgtggtctaccaggtgtcgctggatctgtgggtgaacctggccccctcggcatcgcaggcccacctggggcccgtggtccccctggtaatgtcggtaatcctggcgtcaatggtgctcctggtgaagccggtcgtgacggcaaccctgggaatgacggtcccccaggccgcgatggtcaacccggacacaagggggagcgtggttaccccggtaacgcaggtcctgttggtgctgccggtgctcctggccctcaaggccctgtgggtcccgttggtaaacacggaaaccgtggtgaaccgggtcctgccggtgctgttggtcctgctggtgccgttggcccaagaggtcccagtggcccacaaggtattcgaggtgacaagggagagcctggtgataagggtcccagaggtcttcctggcttaaagggacacaatgggttgcaaggtctcccgggtcttgctggtcatcatggcgatcaaggtgctcccggtgctgtgggtcccgctggtcccaggggccctgctggtccttctggccccgctggcaaagacggtcgcattggacagcctggtgcagtcggacctgctggcattcgtggctctcagggtagccaaggtcctgctggccctcctggtccccctggccctcctggacctcctggcccaagtggtggtggttacgagtttggttttgatggagacttctacagggctgaccagcctcgctcaccaacttctctcagacccaaggattatgaagttgatgctactctgaaatctctcaacaaccagattgagacccttcttactccagaaggctctaggaagaacccagctcgcacatgccgagacttgagactcagccacccagaatggagcagtggttactactggattgaccctaaccaaggatgtactatggatgctatcaaagtatactgtgatttctctactggcgaaacctgcatccgggctcaacctgaagacatcccagtcaagaactggtacagaaattccaaggccaagaagcatgtctgggtaggagaaactatcaacggtggtacccagtttgaatataatgttgaaggagtaaccaccaaggaaatggctacccaacttgccttcatgcgtctgctggccaaccatgcctctcagaacatcacctaccattgcaagaacagcattgcatacatggatgaggaaactggcaacctgaaaaaggctgtcattctgcaaggatccaatgatgtcgaacttgttgccgagggcaacagcagattcacttacactgttcttgtagatggctgctctaaaaagacaaatgaatggcagaagacaatcattgaatataaaacaaacaagccatctcgcctgcctatccttgatattgcacctttggacatcggtggcgctgaccaagaaatcagattgaacattggcccagtctgtttcaaataa

SEQ ID NO: 18 - protein, 1364 aa, synthetic construct
  REGION 1..1364: note = Btau COL1A1
MLSFVDTRTLLLLAVTSCLATCQSLQEATARKGPSGDRGPRGERGPPGPPGRDGDDGIPGPPGPPGPPGPPGLGGNFAAQFDAKGGGPGPMGLMGPRGPPGASGAPGPQGFQGPPGEPGEPGQTGPAGARGPPGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGELGPVGNPGPAGPAGPRGEVGLPGLSGPVGPPGNPGANGLPGAKGAAGLPGVAGAPGLPGPRGIPGPVGAAGATGARGLVGEPGPAGSKGESGNKGEPGAVGQPGPPGPSGEEGKRGSTGEIGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAGARGERGPPGESGAAGPTGPIGSRGPSGPPGPDGNKGEPGVVGAPGTAGPSGPSGLPGERGAAGIPGGKGEKGETGLRGDIGSPGRDGARGAPGAIGAPGPAGANGDRGEAGPAGPAGPAGPRGSPGERGEVGPAGPNGFAGPAGAAGQPGAKGERGTKGPKGENGPVGPTGPVGAAGPSGPNGPPGPAGSRGDGGPPGATGFPGAAGRTGPPGPSGISGPPGPPGPAGKEGLRGPRGDQGPVGRSGETGASGPPGFVGEKGPSGEPGTAGPPGTPGPQGLLGAPGFLGLPGSRGERGLPGVAGSVGEPGPLGIAGPPGARGPPGNVGNPGVNGAPGEAGRDGNPGNDGPPGRDGQPGHKGERGYPGNAGPVGAAGAPGPQGPVGPVGKHGNRGEPGPAGAVGPAGAVGPRGPSGPQGIRGDKGEPGDKGPRGLPGLKGHNGLQGLPGLAGHHGDQGAPGAVGPAGPRGPAGPSGPAGKDGRIGQPGAVGPAGIRGSQGSQGPAGPPGPPGPPGPPGPSGGGYEFGFDGDFYRADQPRSPTSLRPKDYEVDATLKSLNNQIETLLTPEGSRKNPARTCRDLRLSHPEWSSGYYWIDPNQGCTMDAIKVYCDFSTGETCIRAQPEDIPVKNWYRNSKAKKHVWVGETINGGTQFEYNVEGVTTKEMATQLAFMRLLANHASQNITYHCKNSIAYMDEETGNLKKAVILQGSNDVELVAEGNSRFTYTVLVDGCSKKTNEWQKTIIEYKTNKPSRLPILDIAPLDIGGADQEIRLNIGPVCFK

SEQ ID NO: 19 - DNA, 4368 bp, synthetic construct
  misc_feature 1..4368: note = Amis COL1A1
  source 1..4368: mol_type = other DNA
atgttcagctttgtggattctcggttactgctgttgatagcagcgactgtactactcaccaaaggtcaaggagaagaagacattcaaactggaagctgcatacaggatggactagcgtacaacaacacagacgtatggaaacccgagccctgccagatctgcgtatgcgacaatggcaacatcctgtgtgacgatgtcatctgtgatgatacctcggactgtaccaatgctgagatcccctttggagaatgctgtcccatctgtcctgacaccgctggctcttctacctaccccaaatccactggagtagagggtcctaagggagacactggccccagaggacagaggggactcccaggcccacctggcagagatggcattcctggacagcctggtctccctggactcccaggacctccaggccctcctggccttggtggaaacttcgctcctcaaatggcttacggttacggagatgaaaccaaatctgctggcatttctgtccctggacccatgggtccagctggcccccgtggtctccccggcccccctggttctcctggtcctcaaggtttccaaggtcctcctggagagcctggagagcctggtgcttcaggtccaatgggtccccgtggtccagccggcccccctggcaagaacggagatgatggtgaagctggaaagcccggccgtcccggtgagcgcggccctcctggcccccagggtgcacgtggtctgcccggaactgctggcctgccaggcatgaagggtcacagaggtttcagtggtctggatggtgctaagggtgatgctggtccatccggccccaagggtgagcctggtagccctggtgagaacggagctcctggacaaatgggccctcgtggtcttcccggtgagagaggccgccctggtccatctggccctgctggtgctcgtggtaacgatggtagtcctggtgctgctggccctccaggtccaactggcccagctggcccccctggcttccctggtgctgctggtgctaagggtgaaactggtcctcaaggttctcgtggtagtgaaggcccacagggtgctcgtggtgagcctggtcctcctggccctgctggtgctgctggtcctgctggcaaccctggttctgatggtcaagctggtgccaaaggtgcaactggtgctcctggtattgctggtgctcctggcttccctggcgctcgtggcccatctggaccccagggtcccagcggtgctcctggccccaagggtaacagtggtgaacccggtgctcaaggcaacaagggagacactggtgcaaaaggagagcctggtcctgctggtgtccaaggcccacctggtccagctggtgaagaaggcaagagaggagcccgtggtgagcccggccctggaggtcttcctggccctgctggcgaacgtggtgctcctggaagccgtggtttccctggcgctgatggcatttctggtcccaagggtccccctggtgaacgtggttcccctggccctgctggtcccaaaggatctactggtgaatctggacgccctggtgagcctggtctccctggtgccaagggtcttactggaagcccaggtagcccaggtcctgatggcaagactggtccacctggccccgctggtcaagatggtcgcccaggacccccaggcccacctggtgccagaggtcaggctggtgtgatgggtttccctggacctaaaggtgctgctggtgagcctggcaaacctggtgagagaggagctcctggaccccctggtgctgttggcgcagctggtaaggatggtgaagctggtgcccaaggttctcctggcgctgctggtcctgctggagagagaggtgaacaaggtcctgctggtgctcctggattccagggtctgcccggtcctgctggcccatctggtgaatctggcaagcctggtgaacagggtgttcctggagatgctggtgctcctggtccagctggtgcaagaggcgagagaggtttccctggtgagcgtggtgtccaaggtcaaccaggtccacagggtccacgtggtgctaacggtgctcccggtaacgatggtgctaagggtgatgctggtgctcctggtgctcctggtggccaaggtcctcccggtctgcagggtatgcctggtgagcgtggtgctgctggtctgcctggttccaagggtgacagaggcgatcctggtcccaaaggcactgatggtgctcctggcaaagatggcgtcagaggtctaactggccctattggtcctcctggcccagctggtgcccctggtgacaagggtgaagctggtccttctggccctgctggtcccactggttctcgtggtgcccctggagatcgtggtgagcctggtccacctggccctgctggattcgctggtccccctggtgctgatggacaacctggtgctaaaggtgaatctggtgatgctggtgctaaaggtgatgctggtcctccaggccctgctggacccactggtgctcctggaccttctggcgctgttggtgctcctggacccaaaggtgctcgtggtagtgctggaccccctggtgctactggtttccctggtgctgctggaagagttggtccacctggccctgctggtaacgtcggtcttcctggcccatcaggccccagtggaaaagaaggctctaaaggaccccgtggtgagactggccctgctggacgccccggtgaacctggacctgctggcccaccaggaccttctggcgagaagggctctcctggtggtgatggtcccgctggtgctcctggtactccaggcccacagggtattgctggacagcgtggtgtagttggtcttcctggacagagaggcgagagaggtttccctggtctccccggcccatctggcgaacctggcaaacaaggtccatctggctcctctggtgaacgcggtcctcctggtccaatgggaccacctggcttggctggacctcctggtgaagctggacgtgagggtgctcctggttctgaaggtgctcctggtcgcgatggcgctgctggtcccaagggtgaccgtggtgagactggcccctctggtcctcctggtgctcccggtgcccctggagctcctggccctattggccctgctggcaagaatggagatcgtggtgagactggtccttctggtcctgctggccctgccggtcctgctggtgctcgtggtcctgctggtccacaaggtgcccgtggtgacaaaggtgaaactggagaacatggtgacagaggcatgaagggtcacagaggattccctggtccccagggtccctctggtcctgctggctctcctggtgaacaaggtccttctggagcttccggccctgctggtccaagaggtcctcctggctctgctggcacccctggcaaagatggtctgaatggtctccctggccctattggtccacctggtccccggggtcgcactggtgatgttggtcctgctggtccccctggacctcctgggcccccaggtcctcctggtgcacccagcggcggctttgacttcagcttcatgccccagcctcctcaggagaaagcccatgatcctggccgctactacagagctgatgacgccaacgtgatgcgtgaccgtgacctggaggtggacaccaccctcaagagcctgagccagcagatcgagaacatccgcagccccgagggcaccaggaagaaccctgcccgcacctgccgtgacctgaagatgtgccacaatgactggaagagcggcgagtactggattgaccccaaccagggctgcaatctggatgccatcaaggtctactgtaacatggagactggcgagacttgcgtccacccaacccaggccaccatcgctcagaagaactggtacatgagcaagaaccccaaggagaagaaacacatctggtttggcgagacaatgagcgatggcttccagttcgaatatggtggggagggctccaacccagctgacgttgccatccaactgaccttcctgcgcctgatgtccactgaggcctcccagaacatcacctaccactgcaagaacagcgtggcttacatggaccaggagactggcaacctgaagaaggctctgctccttcagggctccaacgagatcgagatcagagcagaaggcaacagccgcttcacctatggagtcactgaggatggctgcacaactcacaccggtgcctggggcaagacagtcattgaatacaaaacaacaaaaacctctcgcctgcccgtcattgacgtggctcccatggacgttggagcacaagatcaggaattcggaattgtcatcggacctgtctgcttcttgtaa

SEQ ID NO: 20 - protein, 1455 aa, synthetic construct
  REGION 1..1455: note = Amis COL1A1
MFSFVDSRLLLLIAATVLLTKGQGEEDIQTGSCIQDGLAYNNTDVWKPEPCQICVCDNGNILCDDVICDDTSDCTNAEIPFGECCPICPDTAGSSTYPKSTGVEGPKGDTGPRGQRGLPGPPGRDGIPGQPGLPGLPGPPGPPGLGGNFAPQMAYGYGDETKSAGISVPGPMGPAGPRGLPGPPGSPGPQGFQGPPGEPGEPGASGPMGPRGPAGPPGKNGDDGEAGKPGRPGERGPPGPQGARGLPGTAGLPGMKGHRGFSGLDGAKGDAGPSGPKGEPGSPGENGAPGQMGPRGLPGERGRPGPSGPAGARGNDGSPGAAGPPGPTGPAGPPGFPGAAGAKGETGPQGSRGSEGPQGARGEPGPPGPAGAAGPAGNPGSDGQAGAKGATGAPGIAGAPGFPGARGPSGPQGPSGAPGPKGNSGEPGAQGNKGDTGAKGEPGPAGVQGPPGPAGEEGKRGARGEPGPGGLPGPAGERGAPGSRGFPGADGISGPKGPPGERGSPGPAGPKGSTGESGRPGEPGLPGAKGLTGSPGSPGPDGKTGPPGPAGQDGRPGPPGPPGARGQAGVMGFPGPKGAAGEPGKPGERGAPGPPGAVGAAGKDGEAGAQGSPGAAGPAGERGEQGPAGAPGFQGLPGPAGPSGESGKPGEQGVPGDAGAPGPAGARGERGFPGERGVQGQPGPQGPRGANGAPGNDGAKGDAGAPGAPGGQGPPGLQGMPGERGAAGLPGSKGDRGDPGPKGTDGAPGKDGVRGLTGPIGPPGPAGAPGDKGEAGPSGPAGPTGSRGAPGDRGEPGPPGPAGFAGPPGADGQPGAKGESGDAGAKGDAGPPGPAGPTGAPGPSGAVGAPGPKGARGSAGPPGATGFPGAAGRVGPPGPAGNVGLPGPSGPSGKEGSKGPRGETGPAGRPGEPGPAGPPGPSGEKGSPGGDGPAGAPGTPGPQGIAGQRGVVGLPGQRGERGFPGLPGPSGEPGKQGPSGSSGERGPPGPMGPPGLAGPPGEAGREGAPGSEGAPGRDGAAGPKGDRGETGPSGPPGAPGAPGAPGPIGPAGKNGDRGETGPSGPAGPAGPAGARGPAGPQGARGDKGETGEHGDRGMKGHRGFPGPQGPSGPAGSPGEQGPSGASGPAGPRGPPGSAGTPGKDGLNGLPGPIGPPGPRGRTGDVGPAGPPGPPGPPGPPGAPSGGFDFSFMPQPPQEKAHDPGRYYRADDANVMRDRDLEVDTTLKSLSQQIENIRSPEGTRKNPARTCRDLKMCHNDWKSGEYWIDPNQGCNLDAIKVYCNMETGETCVHPTQATIAQKNWYMSKNPKEKKHIWFGETMSDGFQFEYGGEGSNPADVAIQLTFLRLMSTEASQNITYHCKNSVAYMDQETGNLKKALLLQGSNEIEIRAEGNSRFTYGVTEDGCTTHTGAWGKTVIEYKTTKTSRLPVIDVAPMDVGAQDQEFGIVIGPVCFL

SEQ ID NO: 21 - DNA, 4092 bp, synthetic construct
  misc_feature 1..4092: note = Amis COL1A2
  source 1..4092: mol_type = other DNA
atgctcagctttgtggatacacggattttgttgctgctcgcagtaacttcgtacctagcaacatgtcaacaagcaaatgaggcaactgcaggacggaagggcccaagaggagacaaagggccacagggagaaaggggtccaccaggtccaccaggcagagatggtgaagatggtccaccagggcctccagggccccctggtcctccaggtcttggcggaaactttgctgctcagtatgacggagcaaaagcaggtgactatggctcaggaccaatgggtttaatgggacccagaggcccacctggaacaagtggacctcctggtcctcctggcttccaaggacctcatggtgagcctggtgaacctggtcaaacaggtccccagggtccccgtggtccatctggtcctcctggaaaggctggtgaagatggccatcctggaaaatctggacgatctggtgagaggggcgtctctggtcctcagggtgctcgtggtttccctggaactcctggtctgcctggctttaagggaattagaggacacaatggtctggatggtcagaagggacaacctggtactccaggcattaagggtgaatccggtgcccctggtgaaaatggtaccccaggacaatctggtgctcgtggccttcccggtgaaagaggaagaattggtgcacctggcccagctggtgcccgtggcagcgatggtagcactggtcccactggtcctgctggccctatcggttctgctggtgctccaggtttcccaggtgctcctggagccaagggtgaaattggagctgctggtaatgtaggtccttctggccctgctggtccacgaggagaggctggacttcctggttcttctggtcccgttggccctcctggaaaccctggttctaatggtcttgctggtgctaaaggtgcaactggtcttcctggtgttgctggtgctcctggcttgcctggtccacgtggtattcctggaccttctggccctgccggagctgctggcaccagaggtcttgttggtgaaccaggccctgctggtgccaagggagaaagtggtaacaagggtgaacccggtgctgctggtccatcaggtcccgctggtccaagtggtgaagaaggcaagaaaggtactactggtgaacctggctcttctggcccccctggtccagctggtctaagaggcgttcctggatctcgtggtctccctggagctgacggcagagctggtgttatgggacctgctggcagccgtggtgctactggtcctgctggtgctaaaggtcctagtggtgataatggtcgccctggtgagcctggccttatgggtccaagaggtctccctggtcaacctggaagctcaggccctgctggcaaggaaggtcctgttggtttccctggtgcagatggtagagttggcccaactggtccagctggtgcaagaggtgagcctggcaacattggattccctggacccaaaggccccactggtgaccctggcaaacctggtgacagaggccatgctggtcttgctggtgctcggggtgcgcctggtcctgagggcaacaatggggctcaaggtcctcctggtgttgctggcaaccctggtgcaaaaggtgaacaaggtccagctggtcctcccggtttccagggtctcccaggcccctcaggtccagctggtgaagctggcaaaccaggtgaaaggggtatggctggtgaatttggtgcccctggccctgcgggttcaagaggtgaacgtggtcctccaggcgaaagtggtgctgttggtcctgtaggtcccattggaagccgtggtccatctggtccaccaggcactgatggcaacaagggtgaacctggtaatgttggtaatgctggtactgcaggcccctctggcgctggtggagccccaggagagagaggcattgctggtattccaggacccaagggtgaaaagggtgctacaggtctgagaggggatactggcgcaacaggaagagatggtgctcgtggtgctcctggtgctattggagcccctggccccgctggtggagctggtgagcggggtgaaggtggtcctgctggtgctgctggcccttctggtgcccgtggtattcctggtgaacgtggtgagcctggtcctgctggccctactggatttgctggacctgctggtgcagctggccaacctggtgctaaaggtgaacgaggtacaaaaggacccaagggtgaaaatggtccacaaggtgctgttggcccagttggttcttctggaccatcaggtcctgttggtgcctctggtcctgctggtcctcgtggtgatggtggtcctcctggtgtcactggtttccctggagctgctggcagaactggtcctcccggcccctctggtatcactggcccccctggtccccctggctcagctggcaaagatggtatgagaggcccacgtggtgatactggtccagttggccgcactggagaacaaggcattgttggcccacctggcttcagtggtgagaaaggtccatctggagagcctggtgctgctggtccccctggtaccccaggtcctcagggtattcttggtgctcctggtatccttggtctgcctggctctcggggagaacgtggtcttccaggcatctctggagcaacaggtgaaccaggtcctcttggtatttccggtcctcctggtgcacgtggtccctctggccccgtgggttctgctggtctgaatggtgcccctggtgaagctggccgtgatggcaatcctggccatgatggtgctccaggccgtgatggtgctcctggtttcaagggtgagcgtggtgctcctgggaacaatggacctgctggtgctgttggtgctcctggcgcccatggtcaagttggtcctgctggaaagcctggaaatcgtggtgatcctggtcctgttggtccttctggtcctgctggtgcttttggtgcaaggggtccttctggcccacaaggtgcacgtggtgagaagggagaaacaggtgaaaagggacacagaggtatgcctggatttaaggggcacaatggacttcagggtctgcctggtcttgctggccaacatggagatcaaggtcctccaggttctactggccccgctggcccaaggggtccctctggtccttctggtcctgctggaaaagatggtcgcaatggactccctggccctattggacctgctggtgtgcgtggttctcagggtagccaaggtccttcgggtccacctggcccacctggtctccctggtccccctggtgcaaatggtggtggatacgaagttggctatgatcttgaatactaccgggctgatcagcctgctctcagacctaaggactatgaagttgatgccactctgaaaacattgaacaaccaaattgagaccctcctgaccccagaaggctccaggaagaacccagctcgcacctgccgtgacctgagactcagccacccagaatggaccagtggtttctactggattgatcccaaccagggctgtactatggatgccattagagtgtattgtgacttctccactggtgagacttgcatacatgccaatctagaaaacatccccactaagaactggtatgtcagcaagaactccaaggaaaagaagcacatgtggtttggtgaaactatcaatggtggtacccagtttgaatataacgatgaaggagtgacttccaaggacatggctacccaacttgccttcatgcgtctgctggccaaccatgcctcccagaacatcacctaccactgcaagaacagtattgcatacatggatgaagaaactggcaaccttaagaaggctgtaatactgcagggatccaatgatgttgaactacgagctgaaggcaacagcagattcactttcagtgttctggaagatggctgctctagaaagaacaacgcatggggcaaaacaatcattgaatatagaacaaacaaaccatctcgcttgcccatccttgacattgcacctttggacattggtggagctgatcaagaattcggtttggacattggcccagtctgtttcaaatga

SEQ ID NO: 22 - protein, 1363 aa, synthetic construct
  REGION 1..1363: note = Amis COL1A2
MLSFVDTRILLLLAVTSYLATCQQANEATAGRKGPRGDKGPQGERGPPGPPGRDGEDGPPGPPGPPGPPGLGGNFAAQYDGAKAGDYGSGPMGLMGPRGPPGTSGPPGPPGFQGPHGEPGEPGQTGPQGPRGPSGPPGKAGEDGHPGKSGRSGERGVSGPQGARGFPGTPGLPGFKGIRGHNGLDGQKGQPGTPGIKGESGAPGENGTPGQSGARGLPGERGRIGAPGPAGARGSDGSTGPTGPAGPIGSAGAPGFPGAPGAKGEIGAAGNVGPSGPAGPRGEAGLPGSSGPVGPPGNPGSNGLAGAKGATGLPGVAGAPGLPGPRGIPGPSGPAGAAGTRGLVGEPGPAGAKGESGNKGEPGAAGPSGPAGPSGEEGKKGTTGEPGSSGPPGPAGLRGVPGSRGLPGADGRAGVMGPAGSRGATGPAGAKGPSGDNGRPGEPGLMGPRGLPGQPGSSGPAGKEGPVGFPGADGRVGPTGPAGARGEPGNIGFPGPKGPTGDPGKPGDRGHAGLAGARGAPGPEGNNGAQGPPGVAGNPGAKGEQGPAGPPGFQGLPGPSGPAGEAGKPGERGMAGEFGAPGPAGSRGERGPPGESGAVGPVGPIGSRGPSGPPGTDGNKGEPGNVGNAGTAGPSGAGGAPGERGIAGIPGPKGEKGATGLRGDTGATGRDGARGAPGAIGAPGPAGGAGERGEGGPAGAAGPSGARGIPGERGEPGPAGPTGFAGPAGAAGQPGAKGERGTKGPKGENGPQGAVGPVGSSGPSGPVGASGPAGPRGDGGPPGVTGFPGAAGRTGPPGPSGITGPPGPPGSAGKDGMRGPRGDTGPVGRTGEQGIVGPPGFSGEKGPSGEPGAAGPPGTPGPQGILGAPGILGLPGSRGERGLPGISGATGEPGPLGISGPPGARGPSGPVGSAGLNGAPGEAGRDGNPGHDGAPGRDGAPGFKGERGAPGNNGPAGAVGAPGAHGQVGPAGKPGNRGDPGPVGPSGPAGAFGARGPSGPQGARGEKGETGEKGHRGMPGFKGHNGLQGLPGLAGQHGDQGPPGSTGPAGPRGPSGPSGPAGKDGRNGLPGPIGPAGVRGSQGSQGPSGPPGPPGLPGPPGANGGGYEVGYDLEYYRADQPALRPKDYEVDATLKTLNNQIETLLTPEGSRKNPARTCRDLRLSHPEWTSGFYWIDPNQGCTMDAIRVYCDFSTGETCIHANLENIPTKNWYVSKNSKEKKHMWFGETINGGTQFEYNDEGVTSKDMATQLAFMRLLANHASQNITYHCKNSIAYMDEETGNLKKAVILQGSNDVELRAEGNSRFTFSVLEDGCSRKNNAWGKTIIEYRTNKPSRLPILDIAPLDIGGADQEFGLDIGPVCFK

SEQ ID NO: 23 - DNA, 90 bp, synthetic construct
  misc_feature 1..90: note = COLsyn1a
  source 1..90: mol_type = other DNA
ggtcctaagggtccaaagggccctaagggacccaaaggtccacctggccctccaggcgatccaggtgaccctggcgaccccggagatcca

SEQ ID NO: 24 - protein, 30 aa, synthetic construct
  REGION 1..30: note = COLsyn1a
GPKGPKGPKGPKGPPGPPGDPGDPGDPGDP

SEQ ID NO: 25 - DNA, 136 bp, synthetic construct
  misc_feature 1..136: note = COLsyn2
  source 1..136: mol_type = other DNA
gcatcgtctcatcggtctcattctggtcctaaaggacccgacggaccaaagggcccagacggaccccctggtccaccaggtgaccccggcaagccaggagatcccggtaaaccaatcctgagacctgagacggcat

SEQ ID NO: 26 - protein, 30 aa, synthetic construct
  REGION 1..30: note = COLsyn2
GPKGPDGPKGPDGPPGPPGDPGKPGDPGKP

SEQ ID NO: 27 - DNA, 90 bp, synthetic construct
  misc_feature 1..90: note = COLsyn3
  source 1..90: mol_type = other DNA
ggaccaaagggacccaaaggaccagacggcccagatggccccccaggacctcctggcgacccaggtgacccaggtaagcctggcaagcct

SEQ ID NO: 28 - protein, 30 aa, synthetic construct
  REGION 1..30: note = COLsyn3
GPKGPKGPDGPDGPPGPPGDPGDPGKPGKP

SEQ ID NO: 29 - DNA, 90 bp, synthetic construct
  misc_feature 1..90: note = COLsyn4
  source 1..90: mol_type = other DNA
ggtcctaaaggaccaaagggtcccaagggcccaaagggtcctccaggagctcctggaccacctggccctccaggtgtcccaggtccacca

SEQ ID NO: 30 - protein, 30 aa, synthetic construct
  REGION 1..30: note = COLsyn4
GPKGPKGPKGPKGPPGAPGPPGPPGVPGPP

SEQ ID NO: 31 - DNA, 90 bp, synthetic construct
  misc_feature 1..90: note = COLsyn5
  source 1..90: mol_type = other DNA
ggtcctgacggacctgatggaccagatggtcctgatggtcctccaggagctcctggaccacctggccctccaggtgtcccaggtccacca

SEQ ID NO: 32 - protein, 30 aa, synthetic construct
  REGION 1..30: note = COLsyn5
GPDGPDGPDGPDGPPGAPGPPGPPGVPGPP

SEQ ID NO: 33 - DNA, 90 bp, synthetic construct
  misc_feature 1..90: note = COLsyn6
  source 1..90: mol_type = other DNA
ggtttagctggtcccccaggtcctgcaggagctcccggtcctccaggagctcctggaccacctggccctccaggtgtcccaggtccacca

SEQ ID NO: 34 - protein, 30 aa, synthetic construct
  REGION 1..30: note = COLsyn6
GLAGPPGPAGAPGPPGAPGPPGPPGVPGPP

SEQ ID NO: 35 - DNA, 877 bp, synthetic construct
  misc_feature 1..877: note = GFP-COLsyn2-ePTS1
  source 1..877: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaagcatcgtctcatcggtctcattctggtcctaaaggacccgacggaccaaagggcccagacggaccccctggtccaccaggtgaccccggcaagccaggagatcccggtaaaccaatcctgagacctgagacggcatttgggaagaggtagaagatccaaattg

SEQ ID NO: 36 - protein, 277 aa, synthetic construct
  REGION 1..277: note = GFP-COLsyn2-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGPKGPDGPKGPDGPPGPPGDPGKPGDPGKPLGRGRRSKL

SEQ ID NO: 37 - DNA, 831 bp, synthetic construct
  misc_feature 1..831: note = GFP-COLsyn3-ePTS1
  source 1..831: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggaccaaagggacccaaaggaccagacggcccagatggccccccaggacctcctggcgacccaggtgacccaggtaagcctggcaagcctttgggaagaggtagaagatccaaattg

SEQ ID NO: 38 - protein, 277 aa, synthetic construct
  REGION 1..277: note = GFP-COLsyn3-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGPKGPKGPDGPDGPPGPPGDPGDPGKPGKPLGRGRRSKL

SEQ ID NO: 39 - DNA, 831 bp, synthetic construct
  misc_feature 1..831: note = GFP-COLsyn6-ePTS1
  source 1..831: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggtttagctggtcccccaggtcctgcaggagctcccggtcctccaggagctcctggaccacctggccctccaggtgtcccaggtccaccattgggaagaggtagaagatccaaattg

SEQ ID NO: 40 - protein, 277 aa, synthetic construct
  REGION 1..277: note = GFP-COLsyn6-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGLAGPPGPAGAPGPPGAPGPPGPPGVPGPPLGRGRRSKL

SEQ ID NO: 41 - DNA, 1605 bp, synthetic construct
  misc_feature 1..1605: note = BtauP4HA1
  source 1..1605: mol_type = other DNA
atgatctggtatattttagttgtagggattctacttccccagtctttggcccatccaggcttttttacttctattggtcagatgactgatttgattcatactgaaaaagatctggtgacttccctgaaagactatataaaggcagaagaggacaaattagaacaaataaaaaaatgggcagagaaattagatcgattaaccagcacagcgacaaaagatccagaaggatttgttggacaccctgtaaatgcattcaaattaatgaaacgtctgaacactgagtggagtgagttggagaatctggtccttaaggatatgtcagatggttttatctctaacctaaccattcagagacagtacttccctaatgatgaagatcaggttggggcagccaaagctctgttgcgtctacaggacacctacaatttggatacagataccatctcaaagggtgatcttccaggagtaaaacacaaatcttttctaacagttgaggactgttttgagttgggcaaagtggcctacacagaagcagattattaccatacagagctgtggatggaacaagcactgaggcagctggatgaaggcgaggtttctaccgttgataaagtctctgttctggattatttgagctatgcagtataccagcagggagacctggataaggcgcttttgctcacaaagaagcttcttgaactagatcctgaacatcagagagctaacggtaacttaaaatactttgagtatataatggctaaagaaaaagatgccaataagtcttcttcagatgaccaatctgatcagaaaaccacactgaagaagaaaggtgctgctgtggattacctgccagagagacagaagtacgaaatgctgtgccgtggggagggtatcaaaatgactcctcggagacagaaaaaactcttctgtcgctaccatgatggaaaccggaatcctaaatttatcctggctccagccaaacaggaggatgagtgggacaagcctcgtattatccgcttccatgatattatttctgatgcagaaattgaagtcgttaaagatctagcaaaaccaaggctgaggcgagccaccatttcaaacccaataacaggagacttggagacggtacattacagaattagcaaaagtgcctggctgtctggctatgaaaaccctgtggtgtcacgaattaatatgagaatccaagatctgacaggactagatgtctccacagcagaggaattacaggtagcaaattatggagttggaggacagtatgaaccccattttgattttgcacggaaagatgagccagatgctttcaaagagctggggacaggaaatagaattgctacatggctgttttatatgagtgatgtgttagcaggaggagccactgtttttcctgaagtaggagctagtgtttggcccaaaaagggaactgctgttttctggtataatctgtttgccagtggagaaggagattatagtacacggcatgcagcctgtccagtgctggttggaaacaaatgggtatccaataaatggctccatgaacgtggacaggaatttcgaagaccatgcaccttgtcagaattggaatga

SEQ ID NO: 42 - protein, 534 aa, synthetic construct
  REGION 1..534: note = BtauP4HA1
MIWYILVVGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGDLPGVKHKSFLTVEDCFELGKVAYTEADYYHTELWMEQALRQLDEGEVSTVDKVSVLDYLSYAVYQQGDLDKALLLTKKLLELDPEHQRANGNLKYFEYIMAKEKDANKSSSDDQSDQKTTLKKKGAAVDYLPERQKYEMLCRGEGIKMTPRRQKKLFCRYHDGNRNPKFILAPAKQEDEWDKPRIIRFHDIISDAEIEVVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVLAGGATVFPEVGASVWPKKGTAVFWYNLFASGEGDYSTRHAACPVLVGNKWVSNKWLHERGQEFRRPCTLSELE

SEQ ID NO: 43 - DNA, 1533 bp, synthetic construct
  misc_feature 1..1533: note = BtauP4HB
  source 1..1533: mol_type = other DNA
atgctgcgccgcgctctgctctgcctggccctgaccgcgctattccgcgcgggtgccggcgcccccgacgaggaggaccacgtcctggtgctccataagggcaacttcgacgaggcgctggcggcccacaagtacctgctggtggagttctacgccccatggtgcggccactgcaaggctctggccccggagtatgccaaagcagctgggaagctgaaggcagaaggttctgagatcagactggccaaggtggatgccactgaagagtctgacctggcccagcagtatggtgtccgaggctaccccaccatcaagttcttcaagaatggagacacagcttcccccaaagagtacacagctggccgagaagcggatgatatcgtgaactggctgaagaagcgcacgggccccgctgccagcacgctgtccgacggggctgctgcagaggccttggtggagtccagtgaggtggccgtcattggcttcttcaaggacatggagtcggactccgcaaagcagttcttcttggcagcagaggtcattgatgacatccccttcgggatcacatctaacagcgatgtgttctccaaataccagctggacaaggatggggttgtcctctttaagaagtttgacgaaggccggaacaactttgagggggaggtcaccaaagaaaagcttctggacttcatcaagcacaaccagttgcccctggtcattgagttcaccgagcagacagccccgaagatcttcggaggggaaatcaagactcacatcctgctgttcctgccgaaaagcgtgtctgactatgagggcaagctgagcaacttcaaaaaagcggctgagagcttcaagggcaagatcctgtttatcttcatcgacagcgaccacactgacaaccagcgcatcctggaattcttcggcctaaagaaagaggagtgcccggccgtgcgcctcatcacgctggaggaggagatgaccaaatataagccagagtcagatgagctgacggcagagaagatcaccgagttctgccaccgcttcctggagggcaagattaagccccacctgatgagccaggagctgcctgacgactgggacaagcagcctgtcaaagtgctggttgggaagaactttgaagaggttgcttttgatgagaaaaagaacgtctttgtagagttctatgccccgtggtgcggtcactgcaagcagctggcccccatctgggataagctgggagagacgtacaaggaccacgagaacatagtcatcgccaagatggactccacggccaacgaggtggaggcggtgaaagtgcacagcttccccacgctcaagttcttccccgccagcgccgacaggacggtcatcgactacaatggggagcggacactggatggttttaagaagttcctggagagtggtggccaggatggggccggagatgatgacgatctagaagatcttgaagaagcagaagagcctgatctggaggaagatgatgatcaaaaagctgtgaaagatgaactgtaa

SEQ ID NO: 44 - protein, 510 aa, synthetic construct
  REGION 1..510: note = BtauP4HB
MLRRALLCLALTALFRAGAGAPDEEDHVLVLHKGNFDEALAAHKYLLVEFYAPWCGHCKALAPEYAKAAGKLKAEGSEIRLAKVDATEESDLAQQYGVRGYPTIKFFKNGDTASPKEYTAGREADDIVNWLKKRTGPAASTLSDGAAAEALVESSEVAVIGFFKDMESDSAKQFFLAAEVIDDIPFGITSNSDVFSKYQLDKDGVVLFKKFDEGRNNFEGEVTKEKLLDFIKHNQLPLVIEFTEQTAPKIFGGEIKTHILLFLPKSVSDYEGKLSNFKKAAESFKGKILFIFIDSDHTDNQRILEFFGLKKEECPAVRLITLEEEMTKYKPESDELTAEKITEFCHRFLEGKIKPHLMSQELPDDWDKQPVKVLVGKNFEEVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDLEEDDDQKAVKDEL

SEQ ID NO: 45 - DNA, 1470 bp, synthetic construct
  misc_feature 1..1470: note = BtP4HB
  source 1..1470: mol_type = other DNA
gcccccgacgaggaggaccacgtcctggtgctccataagggcaacttcgacgaggcgctggcggcccacaagtacctgctggtggagttctacgccccatggtgcggccactgcaaggctctggccccggagtatgccaaagcagctgggaagctgaaggcagaaggttctgagatcagactggccaaggtggatgccactgaagagtctgacctggcccagcagtatggtgtccgaggctaccccaccatcaagttcttcaagaatggagacacagcttcccccaaagagtacacagctggccgagaagcggatgatatcgtgaactggctgaagaagcgcacgggccccgctgccagcacgctgtccgacggggctgctgcagaggccttggtggagtccagtgaggtggccgtcattggcttcttcaaggacatggagtcggactccgcaaagcagttcttcttggcagcagaggtcattgatgacatccccttcgggatcacatctaacagcgatgtgttctccaaataccagctggacaaggatggggttgtcctctttaagaagtttgacgaaggccggaacaactttgagggggaggtcaccaaagaaaagcttctggacttcatcaagcacaaccagttgcccctggtcattgagttcaccgagcagacagccccgaagatcttcggaggggaaatcaagactcacatcctgctgttcctgccgaaaagcgtgtctgactatgagggcaagctgagcaacttcaaaaaagcggctgagagcttcaagggcaagatcctgtttatcttcatcgacagcgaccacactgacaaccagcgcatcctggaattcttcggcctaaagaaagaggagtgcccggccgtgcgcctcatcacgctggaggaggagatgaccaaatataagccagagtcagatgagctgacggcagagaagatcaccgagttctgccaccgcttcctggagggcaagattaagccccacctgatgagccaggagctgcctgacgactgggacaagcagcctgtcaaagtgctggttgggaagaactttgaagaggttgcttttgatgagaaaaagaacgtctttgtagagttctatgccccgtggtgcggtcactgcaagcagctggcccccatctgggataagctgggagagacgtacaaggaccacgagaacatagtcatcgccaagatggactccacggccaacgaggtggaggcggtgaaagtgcacagcttccccacgctcaagttcttccccgccagcgccgacaggacggtcatcgactacaatggggagcggacactggatggttttaagaagttcctggagagtggtggccaggatggggccggagatgatgacgatctagaagatcttgaagaagcagaagagcctgatctggaggaagatgatgatcaaaaagctgtgaaagatgaactg

SEQ ID NO: 46 - protein, 490 aa, synthetic construct
  REGION 1..490: note = BtP4HB
APDEEDHVLVLHKGNFDEALAAHKYLLVEFYAPWCGHCKALAPEYAKAAGKLKAEGSEIRLAKVDATEESDLAQQYGVRGYPTIKFFKNGDTASPKEYTAGREADDIVNWLKKRTGPAASTLSDGAAAEALVESSEVAVIGFFKDMESDSAKQFFLAAEVIDDIPFGITSNSDVFSKYQLDKDGVVLFKKFDEGRNNFEGEVTKEKLLDFIKHNQLPLVIEFTEQTAPKIFGGEIKTHILLFLPKSVSDYEGKLSNFKKAAESFKGKILFIFIDSDHTDNQRILEFFGLKKEECPAVRLITLEEEMTKYKPESDELTAEKITEFCHRFLEGKIKPHLMSQELPDDWDKQPVKVLVGKNFEEVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDLEEDDDQKAVKDEL

SEQ ID NO: 47 - DNA, 2211 bp, synthetic construct
  misc_feature 1..2211: note = GFP-BtP4HB-ePTS1
  source 1..2211: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaagcccccgacgaggaggaccacgtcctggtgctccataagggcaacttcgacgaggcgctggcggcccacaagtacctgctggtggagttctacgccccatggtgcggccactgcaaggctctggccccggagtatgccaaagcagctgggaagctgaaggcagaaggttctgagatcagactggccaaggtggatgccactgaagagtctgacctggcccagcagtatggtgtccgaggctaccccaccatcaagttcttcaagaatggagacacagcttcccccaaagagtacacagctggccgagaagcggatgatatcgtgaactggctgaagaagcgcacgggccccgctgccagcacgctgtccgacggggctgctgcagaggccttggtggagtccagtgaggtggccgtcattggcttcttcaaggacatggagtcggactccgcaaagcagttcttcttggcagcagaggtcattgatgacatccccttcgggatcacatctaacagcgatgtgttctccaaataccagctggacaaggatggggttgtcctctttaagaagtttgacgaaggccggaacaactttgagggggaggtcaccaaagaaaagcttctggacttcatcaagcacaaccagttgcccctggtcattgagttcaccgagcagacagccccgaagatcttcggaggggaaatcaagactcacatcctgctgttcctgccgaaaagcgtgtctgactatgagggcaagctgagcaacttcaaaaaagcggctgagagcttcaagggcaagatcctgtttatcttcatcgacagcgaccacactgacaaccagcgcatcctggaattcttcggcctaaagaaagaggagtgcccggccgtgcgcctcatcacgctggaggaggagatgaccaaatataagccagagtcagatgagctgacggcagagaagatcaccgagttctgccaccgcttcctggagggcaagattaagccccacctgatgagccaggagctgcctgacgactgggacaagcagcctgtcaaagtgctggttgggaagaactttgaagaggttgcttttgatgagaaaaagaacgtctttgtagagttctatgccccgtggtgcggtcactgcaagcagctggcccccatctgggataagctgggagagacgtacaaggaccacgagaacatagtcatcgccaagatggactccacggccaacgaggtggaggcggtgaaagtgcacagcttccccacgctcaagttcttccccgccagcgccgacaggacggtcatcgactacaatggggagcggacactggatggttttaagaagttcctggagagtggtggccaggatggggccggagatgatgacgatctagaagatcttgaagaagcagaagagcctgatctggaggaagatgatgatcaaaaagctgtgaaagatgaactgttgggaagaggtagaagatccaaattg

SEQ ID NO: 48 - protein, 737 aa, synthetic construct
  REGION 1..737: note = GFP-BtP4HB-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKAPDEEDHVLVLHKGNFDEALAAHKYLLVEFYAPWCGHCKALAPEYAKAAGKLKAEGSEIRLAKVDATEESDLAQQYGVRGYPTIKFFKNGDTASPKEYTAGREADDIVNWLKKRTGPAASTLSDGAAAEALVESSEVAVIGFFKDMESDSAKQFFLAAEVIDDIPFGITSNSDVFSKYQLDKDGVVLFKKFDEGRNNFEGEVTKEKLLDFIKHNQLPLVIEFTEQTAPKIFGGEIKTHILLFLPKSVSDYEGKLSNFKKAAESFKGKILFIFIDSDHTDNQRILEFFGLKKEECPAVRLITLEEEMTKYKPESDELTAEKITEFCHRFLEGKIKPHLMSQELPDDWDKQPVKVLVGKNFEEVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDLEEDDDQKAVKDELLGRGRRSKL

SEQ ID NO: 49 - DNA, 708 bp, synthetic construct
  misc_feature 1..708: note = TEV protease
  source 1..708: mol_type = other DNA
ggagagtccctgtttaaaggacccagagactataacccgattagtagcactatttgtcatcttacaaacgaaagtgatggtcacacgactagtctttacggaatcggattcggcccatttattatcacaaacaagcatctgttcagaagaaataacgggacgttgttggtccaatctcttcatggagtatttaaggtaaagaacactacaactcttcagcagcatctgatcgacggtagggatatgatcatcatccgtatgccgaaagactttccaccttttcctcagaagttgaagtttagagaaccccagcgtgaggagcgtatctgtttagtaacaacaaatttccaaacgaaatctatgtcatcaatggttagcgataccagttgtactttccccagttcagatgggattttctggaagcactggattcagacaaaggacggtcagtgtggtagtccgcttgtttctacaagggacggatttattgtcgggatacacagtgcttctaactttacgaatacaaacaactacttcacgtctgtccctaaaaattttatggagctgttgactaatcaggaagcccaacagtgggtatctggctggcgtttgaacgcggattccgtactgtggggtggccacaaggtttttatggttaagcctgaagagccgttccaacctgtgaaggaggcaacacagctaatgaat

SEQ ID NO: 50 - protein, 236 aa, synthetic construct
  REGION 1..236: note = TEV protease
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 51 - DNA, 1182 bp, synthetic construct
  misc_feature 1..1182: note = mRuby-BantP4H-ePTS1
  source 1..1182: mol_type = other DNA
atggtgtccaaaggagaggagttaatcaaggaaaacatgagaatgaaagttgtcatggagggctccgttaatggtcaccaattcaagtgtacaggggaaggtgaaggtaatccttacatgggtacacaaactatgagaattaaagtaattgaaggcggaccactaccatttgcatttgacattctggcaacgtcattcatgtacggatcacgaactttcatcaagtaccctaaaggtataccagactttttcaagcaatcttttccagagggttttacatgggaaagggttacaagatacgaagatgggggtgtcgtcacagttatgcaagatacttcattagaagatggctgccttgtctatcatgtgcaagtaagaggggtgaattttccttctaacggacctgtgatgcagaaaaagaccaaaggttgggaaccaaatactgaaatgatgtacccagctgatggaggtttgagaggctacacacacatggcgcttaaagttgatggtggaggtcatttgtcttgtagttttgttaccacttatcgttctaaaaagactgttggcaatatcaaaatgccaggaatacatgctgtagaccacagactagaaagactcgaagagagcgataacgaaatgttcgttgtacagagagagcatgccgtagccaaatttgctggcttaggcggtggtatggatgaattgtataagggttctaaaactttaggctatatccttatggagaatggcgagaagattgacttggagttctttcccgaggaagcacctaaaacagtagaaaacttcaaaaagctggctgagcaaggtttctacgatggtgttacattccacagggttatcccaggattcgtgtcacaaggtggagatccaaccggtacgggagctggcggtccaggttactcaatcccatgtgaaacggacggcaatccccataggcaccttgttggcagtctaagtatggcacatgctggtcgtaacaccggcggttcacagttcttcatagtacacgaaccccaaccccaccttgacggagtgcacacagtctttggtaaggctacctcaggtatcgaaacggtactaaatatgaggcaaggtgacgtaatgaaagaggtcaaggtctgggaggaaggatccttgggaagaggtagaagatccaaattg

SEQ ID NO: 52 - protein, 394 aa, synthetic construct
  REGION 1..394: note = mRuby-BantP4H-ePTS1
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYKGSKTLGYILMENGEKIDLEFFPEEAPKTVENFKKLAEQGFYDGVTFHRVIPGFVSQGGDPTGTGAGGPGYSIPCETDGNPHRHLVGSLSMAHAGRNTGGSQFFIVHEPQPHLDGVHTVFGKATSGIETVLNMRQGDVMKEVKVWEEGSLGRGRRSKL

SEQ ID NO: 53 - DNA, 1053 bp, synthetic construct
  misc_feature 1..1053: note = GFP-BTCol1A1 403-0P-ePTS1
  source 1..1053: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctggatttgctggccctaaaggagccgcaggagaggctggtaaagcaggcgaacgtggcgtagctggcccagctggtgctgtgggtccagttggtaaagatggagaggccggtgcccaaggtccagccggtcctgtaggcccagcaggtgaaagaggagaacaaggtcccgcaggctcagctggattccaaggattagcaggacctgccggcccagttggcgaagccggtaaagccggagagcaaggcgtggctggtgacctaggtgcagttggccctagtggcgccaggggagaaagaggatccttgggaagaggtagaagatccaaattgtaa

SEQ ID NO: 54 - protein, 350 aa, synthetic construct
  REGION 1..350: note = GFP-BTCol1A1 403-0P-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGFAGPKGAAGEAGKAGERGVAGPAGAVGPVGKDGEAGAQGPAGPVGPAGERGEQGPAGSAGFQGLAGPAGPVGEAGKAGEQGVAGDLGAVGPSGARGERGSLGRGRRSKL

SEQ ID NO: 55 - DNA, 1053 bp, synthetic construct
  misc_feature 1..1053: note = GFP-BTCol1A1 403-11P-ePTS1
  source 1..1053: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctggatttcctggccctaagggagccgcaggagagcccggtaaagcaggcgaaagaggcgtacctggtccacccggtgctgtgggtccagctggtaaagatggagaggccggtgcccaaggtcctcctggtcctgctggcccagcaggtgaaagaggagaacaaggtcccgcaggctcacctggattccaaggattaccaggtccagccggaccacctggcgaagccggtaaacccggagagcaaggcgtgcctggtgacctaggtgcaccaggacctagtggcgccaggggagaaagaggatccttgggaagaggtagaagatccaaattgtaa

SEQ ID NO: 56 - protein, 350 aa, synthetic construct
  REGION 1..350: note = GFP-BTCol1A1 403-11P-ePTS1
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGSLGRGRRSKL

SEQ ID NO: 57 - DNA, 5094 bp, synthetic construct
  misc_feature 1..5094: note = AmisCOL1A1-TEV-GFP-HIS-ePTS1
  source 1..5094: mol_type = other DNA
atgcaaggagaagaagacattcaaactggaagctgcatacaggatggactagcgtacaacaacacagacgtatggaaacccgagccctgccagatctgcgtatgcgacaatggcaacatcctgtgtgacgatgtcatctgtgatgatacctcggactgtaccaatgctgagatcccctttggagaatgctgtcccatctgtcctgacaccgctggctcttctacctaccccaaatccactggagtagagggtcctaagggagacactggccccagaggacagaggggactcccaggcccacctggcagagatggcattcctggacagcctggtctccctggactcccaggacctccaggccctcctggccttggtggaaacttcgctcctcaaatggcttacggttacggagatgaaaccaaatctgctggcatttctgtccctggacccatgggtccagctggcccccgtggtctccccggcccccctggttctcctggtcctcaaggtttccaaggtcctcctggagagcctggagagcctggtgcttcaggtccaatgggtccccgtggtccagccggcccccctggcaagaacggagatgatggtgaagctggaaagcccggccgtcccggtgagcgcggccctcctggcccccagggtgcacgtggtctgcccggaactgctggcctgccaggcatgaagggtcacagaggtttcagtggtctggatggtgctaagggtgatgctggtccatccggccccaagggtgagcctggtagccctggtgagaacggagctcctggacaaatgggccctcgtggtcttcccggtgagagaggccgccctggtccatctggccctgctggtgctcgtggtaacgatggtagtcctggtgctgctggccctccaggtccaactggcccagctggcccccctggcttccctggtgctgctggtgctaagggtgaaactggtcctcaaggttctcgtggtagtgaaggcccacagggtgctcgtggtgagcctggtcctcctggccctgctggtgctgctggtcctgctggcaaccctggttctgatggtcaagctggtgccaaaggtgcaactggtgctcctggtattgctggtgctcctggcttccctggcgctcgtggcccatctggaccccagggtcccagcggtgctcctggccccaagggtaacagtggtgaacccggtgctcaaggcaacaagggagacactggtgcaaaaggagagcctggtcctgctggtgtccaaggcccacctggtccagctggtgaagaaggcaagagaggagcccgtggtgagcccggccctggaggtcttcctggccctgctggcgaacgtggtgctcctggaagccgtggtttccctggcgctgatggcatttctggtcccaagggtccccctggtgaacgtggttcccctggccctgctggtcccaaaggatctactggtgaatctggacgccctggtgagcctggtctccctggtgccaagggtcttactggaagcccaggtagcccaggtcctgatggcaagactggtccacctggccccgctggtcaagatggtcgcccaggacccccaggcccacctggtgccagaggtcaggctggtgtgatgggtttccctggacctaaaggtgctgctggtgagcctggcaaacctggtgagagaggagctcctggaccccctggtgctgttggcgcagctggtaaggatggtgaagctggtgcccaaggttctcctggcgctgctggtcctgctggagagagaggtgaacaaggtcctgctggtgctcctggattccagggtctgcccggtcctgctggcccatctggtgaatctggcaagcctggtgaacagggtgttcctggagatgctggtgctcctggtccagctggtgcaagaggcgagagaggtttccctggtgagcgtggtgtccaaggtcaaccaggtccacagggtccacgtggtgctaacggtgctcccggtaacgatggtgctaagggtgatgctggtgctcctggtgctcctggtggccaaggtcctcccggtctgcagggtatgcctggtgagcgtggtgctgctggtctgcctggttccaagggtgacagaggcgatcctggtcccaaaggcactgatggtgctcctggcaaagatggcgtcagaggtctaactggccctattggtcctcctggcccagctggtgcccctggtgacaagggtgaagctggtccttctggccctgctggtcccactggttctcgtggtgcccctggagatcgtggtgagcctggtccacctggccctgctggattcgctggtccccctggtgctgatggacaacctggtgctaaaggtgaatctggtgatgctggtgctaaaggtgatgctggtcctccaggccctgctggacccactggtgctcctggaccttctggcgctgttggtgctcctggacccaaaggtgctcgtggtagtgctggaccccctggtgctactggtttccctggtgctgctggaagagttggtccacctggccctgctggtaacgtcggtcttcctggcccatcaggccccagtggaaaagaaggctctaaaggaccccgtggtgagactggccctgctggacgccccggtgaacctggacctgctggcccaccaggaccttctggcgagaagggctctcctggtggtgatggtcccgctggtgctcctggtactccaggcccacagggtattgctggacagcgtggtgtagttggtcttcctggacagagaggcgagagaggtttccctggtctccccggcccatctggcgaacctggcaaacaaggtccatctggctcctctggtgaacgcggtcctcctggtccaatgggaccacctggcttggctggacctcctggtgaagctggacgtgagggtgctcctggttctgaaggtgctcctggtcgcgatggcgctgctggtcccaagggtgaccgtggtgagactggcccctctggtcctcctggtgctcccggtgcccctggagctcctggccctattggccctgctggcaagaatggagatcgtggtgagactggtccttctggtcctgctggccctgccggtcctgctggtgctcgtggtcctgctggtccacaaggtgcccgtggtgacaaaggtgaaactggagaacatggtgacagaggcatgaagggtcacagaggattccctggtccccagggtccctctggtcctgctggctctcctggtgaacaaggtccttctggagcttccggccctgctggtccaagaggtcctcctggctctgctggcacccctggcaaagatggtctgaatggtctccctggccctattggtccacctggtccccggggtcgcactggtgatgttggtcctgctggtccccctggacctcctgggcccccaggtcctcctggtgcacccagcggcggctttgacttcagcttcatgccccagcctcctcaggagaaagcccatgatcctggccgctactacagagctgatgacgccaacgtgatgcgtgaccgtgacctggaggtggacaccaccctcaagagcctgagccagcagatcgagaacatccgcagccccgagggcaccaggaagaaccctgcccgcacctgccgtgacctgaagatgtgccacaatgactggaagagcggcgagtactggattgaccccaaccagggctgcaatctggatgccatcaaggtctactgtaacatggagactggcgagacttgcgtccacccaacccaggccaccatcgctcagaagaactggtacatgagcaagaaccccaaggagaagaaacacatctggtttggcgagacaatgagcgatggcttccagttcgaatatggtggggagggctccaacccagctgacgttgccatccaactgaccttcctgcgcctgatgtccactgaggcctcccagaacatcacctaccactgcaagaacagcgtggcttacatggaccaggagactggcaacctgaagaaggctctgctccttcagggctccaacgagatcgagatcagagcagaaggcaacagccgcttcacctatggagtcactgaggatggctgcacaactcacaccggtgcctggggcaagacagtcattgaatacaaaacaacaaaaacctctcgcctgcccgtcattgacgtggctcccatggacgttggagcacaagatcaggaattcggaattgtcatcggacctgtctgcttcttgggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 58 - protein, 1697 aa, synthetic construct
  REGION 1..1697: note = AmisCOL1A1-TEV-GFP-HIS-ePTS1
MQGEEDIQTGSCIQDGLAYNNTDVWKPEPCQICVCDNGNILCDDVICDDTSDCTNAEIPFGECCPICPDTAGSSTYPKSTGVEGPKGDTGPRGQRGLPGPPGRDGIPGQPGLPGLPGPPGPPGLGGNFAPQMAYGYGDETKSAGISVPGPMGPAGPRGLPGPPGSPGPQGFQGPPGEPGEPGASGPMGPRGPAGPPGKNGDDGEAGKPGRPGERGPPGPQGARGLPGTAGLPGMKGHRGFSGLDGAKGDAGPSGPKGEPGSPGENGAPGQMGPRGLPGERGRPGPSGPAGARGNDGSPGAAGPPGPTGPAGPPGFPGAAGAKGETGPQGSRGSEGPQGARGEPGPPGPAGAAGPAGNPGSDGQAGAKGATGAPGIAGAPGFPGARGPSGPQGPSGAPGPKGNSGEPGAQGNKGDTGAKGEPGPAGVQGPPGPAGEEGKRGARGEPGPGGLPGPAGERGAPGSRGFPGADGISGPKGPPGERGSPGPAGPKGSTGESGRPGEPGLPGAKGLTGSPGSPGPDGKTGPPGPAGQDGRPGPPGPPGARGQAGVMGFPGPKGAAGEPGKPGERGAPGPPGAVGAAGKDGEAGAQGSPGAAGPAGERGEQGPAGAPGFQGLPGPAGPSGESGKPGEQGVPGDAGAPGPAGARGERGFPGERGVQGQPGPQGPRGANGAPGNDGAKGDAGAPGAPGGQGPPGLQGMPGERGAAGLPGSKGDRGDPGPKGTDGAPGKDGVRGLTGPIGPPGPAGAPGDKGEAGPSGPAGPTGSRGAPGDRGEPGPPGPAGFAGPPGADGQPGAKGESGDAGAKGDAGPPGPAGPTGAPGPSGAVGAPGPKGARGSAGPPGATGFPGAAGRVGPPGPAGNVGLPGPSGPSGKEGSKGPRGETGPAGRPGEPGPAGPPGPSGEKGSPGGDGPAGAPGTPGPQGIAGQRGVVGLPGQRGERGFPGLPGPSGEPGKQGPSGSSGERGPPGPMGPPGLAGPPGEAGREGAPGSEGAPGRDGAAGPKGDRGETGPSGPPGAPGAPGAPGPIGPAGKNGDRGETGPSGPAGPAGPAGARGPAGPQGARGDKGETGEHGDRGMKGHRGFPGPQGPSGPAGSPGEQGPSGASGPAGPRGPPGSAGTPGKDGLNGLPGPIGPPGPRGRTGDVGPAGPPGPPGPPGPPGAPSGGFDFSFMPQPPQEKAHDPGRYYRADDANVMRDRDLEVDTTLKSLSQQIENIRSPEGTRKNPARTCRDLKMCHNDWKSGEYWIDPNQGCNLDAIKVYCNMETGETCVHPTQATIAQKNWYMSKNPKEKKHIWFGETMSDGFQFEYGGEGSNPADVAIQLTFLRLMSTEASQNITYHCKNSVAYMDQETGNLKKALLLQGSNEIEIRAEGNSRFTYGVTEDGCTTHTGAWGKTVIEYKTTKTSRLPVIDVAPMDVGAQDQEFGIVIGPVCFLGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 59 - DNA, 4818 bp, synthetic construct
  misc_feature 1..4818: note = AmisCOL1A2-TEV-GFP-HIS-ePTS1
  source 1..4818: mol_type = other DNA
atgcaacaagcaaatgaggcaactgcaggacggaagggcccaagaggagacaaagggccacagggagaaaggggtccaccaggtccaccaggcagagatggtgaagatggtccaccagggcctccagggccccctggtcctccaggtcttggcggaaactttgctgctcagtatgacggagcaaaagcaggtgactatggctcaggaccaatgggtttaatgggacccagaggcccacctggaacaagtggacctcctggtcctcctggcttccaaggacctcatggtgagcctggtgaacctggtcaaacaggtccccagggtccccgtggtccatctggtcctcctggaaaggctggtgaagatggccatcctggaaaatctggacgatctggtgagaggggcgtctctggtcctcagggtgctcgtggtttccctggaactcctggtctgcctggctttaagggaattagaggacacaatggtctggatggtcagaagggacaacctggtactccaggcattaagggtgaatccggtgcccctggtgaaaatggtaccccaggacaatctggtgctcgtggccttcccggtgaaagaggaagaattggtgcacctggcccagctggtgcccgtggcagcgatggtagcactggtcccactggtcctgctggccctatcggttctgctggtgctccaggtttcccaggtgctcctggagccaagggtgaaattggagctgctggtaatgtaggtccttctggccctgctggtccacgaggagaggctggacttcctggttcttctggtcccgttggccctcctggaaaccctggttctaatggtcttgctggtgctaaaggtgcaactggtcttcctggtgttgctggtgctcctggcttgcctggtccacgtggtattcctggaccttctggccctgccggagctgctggcaccagaggtcttgttggtgaaccaggccctgctggtgccaagggagaaagtggtaacaagggtgaacccggtgctgctggtccatcaggtcccgctggtccaagtggtgaagaaggcaagaaaggtactactggtgaacctggctcttctggcccccctggtccagctggtctaagaggcgttcctggatctcgtggtctccctggagctgacggcagagctggtgttatgggacctgctggcagccgtggtgctactggtcctgctggtgctaaaggtcctagtggtgataatggtcgccctggtgagcctggccttatgggtccaagaggtctccctggtcaacctggaagctcaggccctgctggcaaggaaggtcctgttggtttccctggtgcagatggtagagttggcccaactggtccagctggtgcaagaggtgagcctggcaacattggattccctggacccaaaggccccactggtgaccctggcaaacctggtgacagaggccatgctggtcttgctggtgctcggggtgcgcctggtcctgagggcaacaatggggctcaaggtcctcctggtgttgctggcaaccctggtgcaaaaggtgaacaaggtccagctggtcctcccggtttccagggtctcccaggcccctcaggtccagctggtgaagctggcaaaccaggtgaaaggggtatggctggtgaatttggtgcccctggccctgcgggttcaagaggtgaacgtggtcctccaggcgaaagtggtgctgttggtcctgtaggtcccattggaagccgtggtccatctggtccaccaggcactgatggcaacaagggtgaacctggtaatgttggtaatgctggtactgcaggcccctctggcgctggtggagccccaggagagagaggcattgctggtattccaggacccaagggtgaaaagggtgctacaggtctgagaggggatactggcgcaacaggaagagatggtgctcgtggtgctcctggtgctattggagcccctggccccgctggtggagctggtgagcggggtgaaggtggtcctgctggtgctgctggcccttctggtgcccgtggtattcctggtgaacgtggtgagcctggtcctgctggccctactggatttgctggacctgctggtgcagctggccaacctggtgctaaaggtgaacgaggtacaaaaggacccaagggtgaaaatggtccacaaggtgctgttggcccagttggttcttctggaccatcaggtcctgttggtgcctctggtcctgctggtcctcgtggtgatggtggtcctcctggtgtcactggtttccctggagctgctggcagaactggtcctcccggcccctctggtatcactggcccccctggtccccctggctcagctggcaaagatggtatgagaggcccacgtggtgatactggtccagttggccgcactggagaacaaggcattgttggcccacctggcttcagtggtgagaaaggtccatctggagagcctggtgctgctggtccccctggtaccccaggtcctcagggtattcttggtgctcctggtatccttggtctgcctggctctcggggagaacgtggtcttccaggcatctctggagcaacaggtgaaccaggtcctcttggtatttccggtcctcctggtgcacgtggtccctctggccccgtgggttctgctggtctgaatggtgcccctggtgaagctggccgtgatggcaatcctggccatgatggtgctccaggccgtgatggtgctcctggtttcaagggtgagcgtggtgctcctgggaacaatggacctgctggtgctgttggtgctcctggcgcccatggtcaagttggtcctgctggaaagcctggaaatcgtggtgatcctggtcctgttggtccttctggtcctgctggtgcttttggtgcaaggggtccttctggcccacaaggtgcacgtggtgagaagggagaaacaggtgaaaagggacacagaggtatgcctggatttaaggggcacaatggacttcagggtctgcctggtcttgctggccaacatggagatcaaggtcctccaggttctactggccccgctggcccaaggggtccctctggtccttctggtcctgctggaaaagatggtcgcaatggactccctggccctattggacctgctggtgtgcgtggttctcagggtagccaaggtccttcgggtccacctggcccacctggtctccctggtccccctggtgcaaatggtggtggatacgaagttggctatgatcttgaatactaccgggctgatcagcctgctctcagacctaaggactatgaagttgatgccactctgaaaacattgaacaaccaaattgagaccctcctgaccccagaaggctccaggaagaacccagctcgcacctgccgtgacctgagactcagccacccagaatggaccagtggtttctactggattgatcccaaccagggctgtactatggatgccattagagtgtattgtgacttctccactggtgagacttgcatacatgccaatctagaaaacatccccactaagaactggtatgtcagcaagaactccaaggaaaagaagcacatgtggtttggtgaaactatcaatggtggtacccagtttgaatataacgatgaaggagtgacttccaaggacatggctacccaacttgccttcatgcgtctgctggccaaccatgcctcccagaacatcacctaccactgcaagaacagtattgcatacatggatgaagaaactggcaaccttaagaaggctgtaatactgcagggatccaatgatgttgaactacgagctgaaggcaacagcagattcactttcagtgttctggaagatggctgctctagaaagaacaacgcatggggcaaaacaatcattgaatatagaacaaacaaaccatctcgcttgcccatccttgacattgcacctttggacattggtggagctgatcaagaattcggtttggacattggcccagtctgtttcaaaggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 60 - protein, 1605 aa, synthetic construct
  REGION 1..1605: note = AmisCOL1A2-TEV-GFP-HIS-ePTS1
MQQANEATAGRKGPRGDKGPQGERGPPGPPGRDGEDGPPGPPGPPGPPGLGGNFAAQYDGAKAGDYGSGPMGLMGPRGPPGTSGPPGPPGFQGPHGEPGEPGQTGPQGPRGPSGPPGKAGEDGHPGKSGRSGERGVSGPQGARGFPGTPGLPGFKGIRGHNGLDGQKGQPGTPGIKGESGAPGENGTPGQSGARGLPGERGRIGAPGPAGARGSDGSTGPTGPAGPIGSAGAPGFPGAPGAKGEIGAAGNVGPSGPAGPRGEAGLPGSSGPVGPPGNPGSNGLAGAKGATGLPGVAGAPGLPGPRGIPGPSGPAGAAGTRGLVGEPGPAGAKGESGNKGEPGAAGPSGPAGPSGEEGKKGTTGEPGSSGPPGPAGLRGVPGSRGLPGADGRAGVMGPAGSRGATGPAGAKGPSGDNGRPGEPGLMGPRGLPGQPGSSGPAGKEGPVGFPGADGRVGPTGPAGARGEPGNIGFPGPKGPTGDPGKPGDRGHAGLAGARGAPGPEGNNGAQGPPGVAGNPGAKGEQGPAGPPGFQGLPGPSGPAGEAGKPGERGMAGEFGAPGPAGSRGERGPPGESGAVGPVGPIGSRGPSGPPGTDGNKGEPGNVGNAGTAGPSGAGGAPGERGIAGIPGPKGEKGATGLRGDTGATGRDGARGAPGAIGAPGPAGGAGERGEGGPAGAAGPSGARGIPGERGEPGPAGPTGFAGPAGAAGQPGAKGERGTKGPKGENGPQGAVGPVGSSGPSGPVGASGPAGPRGDGGPPGVTGFPGAAGRTGPPGPSGITGPPGPPGSAGKDGMRGPRGDTGPVGRTGEQGIVGPPGFSGEKGPSGEPGAAGPPGTPGPQGILGAPGILGLPGSRGERGLPGISGATGEPGPLGISGPPGARGPSGPVGSAGLNGAPGEAGRDGNPGHDGAPGRDGAPGFKGERGAPGNNGPAGAVGAPGAHGQVGPAGKPGNRGDPGPVGPSGPAGAFGARGPSGPQGARGEKGETGEKGHRGMPGFKGHNGLQGLPGLAGQHGDQGPPGSTGPAGPRGPSGPSGPAGKDGRNGLPGPIGPAGVRGSQGSQGPSGPPGPPGLPGPPGANGGGYEVGYDLEYYRADQPALRPKDYEVDATLKTLNNQIETLLTPEGSRKNPARTCRDLRLSHPEWTSGFYWIDPNQGCTMDAIRVYCDFSTGETCIHANLENIPTKNWYVSKNSKEKKHMWFGETINGGTQFEYNDEGVTSKDMATQLAFMRLLANHASQNITYHCKNSIAYMDEETGNLKKAVILQGSNDVELRAEGNSRFTFSVLEDGCSRKNNAWGKTIIEYRTNKPSRLPILDIAPLDIGGADQEFGLDIGPVCFKGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 61 - DNA, 5118 bp, synthetic construct
  misc_feature 1..5118: note = BtCOL1A1-TEV-GFP-HIS-ePTS1
  source 1..5118: mol_type = other DNA
atgcaagaggagggccaggaagaaggccaagaagaagacatcccaccagtcacctgcgtacagaacggcctcaggtaccatgaccgagacgtgtggaaacccgtgccctgccagatctgtgtctgcgacaacggcaacgtgctgtgcgatgacgtgatctgcgacgaacttaaggactgtcctaacgccaaagtccccacggacgaatgctgccccgtctgccccgaaggccaggaatcacccacggaccaagaaaccaccggagtcgagggaccgaaaggagacactggcccccgaggcccaaggggacccgccggcccccccggccgagatggcatccctggacaacctggacttcccggaccccctggaccccccggacctcccggaccccctggcctcggaggaaactttgctccccagttgtcttacggctatgatgagaaatcaacaggaatttccgtgcctggtcccatgggtccttctggtcctcgtggtctccctggcccccctggcgcacctggtccccaaggtttccaaggcccccctggtgagcctggcgagccaggagcctcaggtcccatgggtccccgtggtccccctggcccccctggcaagaacggagatgatggcgaagctggaaagcctggtcgtcctggtgagcgcgggcctcccggacctcagggtgctcggggattgcctggaacagctggcctccctggaatgaagggacacagaggtttcagtggtttggatggtgccaagggagatgctggtcctgctggccccaagggcgagcctggtagccccggtgaaaatggagctcctggtcagatgggcccccgtggtctgcctggtgagagaggtcgccctggagcccctggccctgctggtgctcgaggaaatgatggtgcgactggtgctgctgggccccctggtcccactggccccgctggtcctcctggtttccctggtgctgtgggtgctaagggtgaaggtggtccccaaggaccccgaggttctgaaggtccccagggtgtacgtggtgagcctggcccccctggccctgctggtgctgctggccctgctggcaaccctggtgctgatggacagcctggtgctaaaggagccaatggcgctcctggtattgctggtgctcctggcttccctggtgcccgaggcccctctggaccccagggccccagcggcccccctggccccaagggtaacagcggtgaacctggtgctcctggcagcaaaggagacactggcgccaagggagaacccggtcccactggtattcaaggcccccctggccccgctggggaagaaggaaagcgaggagcccgaggtgaacctggacctgctggcctgcctggaccccctggcgagcgtggtggacctggaagccgtggtttccctggcgccgacggtgttgctggtcccaagggtcctgctggtgaacgcggtgctcctggccctgctggccccaaaggttctcctggtgaagctggtcgccccggtgaagctggtctgcccggtgccaagggtctgactggaagccctggcagcccgggtcctgatggcaaaactggcccccctggtcccgccggtcaagatggccgccctggacctccaggccctcccggtgcccgtggtcaggctggcgtgatgggtttccctggacctaaaggtgctgctggagagcctggaaaagctggagagcgaggtgttcctggaccccctggcgctgttggtcctgctggcaaagacggagaagctggagctcagggacccccaggacctgctggccccgctggtgagagaggcgaacaaggccctgctggctcccctggattccagggtctccccggccctgctggtcctcctggtgaagcaggcaaacctggtgaacagggtgttcctggagatcttggtgcccccggcccctctggagcaagaggcgagagaggtttccccggcgagcgtggtgtgcaagggccgcccggtcctgcaggtccccgtggggccaatggtgcccctggcaacgatggtgctaagggtgatgctggtgcccctggagcccccggtagccagggtgcccctggccttcaaggaatgcctggtgaacgaggtgcagctggtcttccaggccctaagggtgacagaggggatgctggtcccaaaggtgctgatggtgctcctggcaaagatggcgtccgtggtctgactggtcccatcggtcctcctggccccgctggtgcccctggtgacaagggtgaagctggtcctagtggcccagccggtcccactggagctcgtggtgcccccggtgaccgtggtgagcctggtccccccggccctgctggcttcgctggcccccctggtgctgatggccaacctggtgctaaaggcgaacctggtgatgctggtgctaaaggtgacgctggtccccccggccctgctgggcccgctggaccccccggccccattggtaacgttggtgctcccggacccaaaggtgctcgtggcagcgctggtccccctggtgctactggtttcccaggtgctgctggccgagtcggtccccccggcccctctggaaatgctggaccccctggccctcctggccctgctggcaaagaaggcagcaaaggcccccgcggtgagactggccccgctgggcgtcccggtgaagtcggtccccctggtccccctggccccgctggtgagaaaggagcccctggtgctgacggacctgctggagctcctggcactcctggacctcaaggtattgctggacagcgtggtgtggtcggcctgcctggtcagagaggagaaagaggcttccctggtcttcctggcccctctggtgaacccggcaaacaaggtccttctggagcaagtggtgaacgtggcccccctggtcccatgggcccccctggattggctggaccccctggcgagtctggacgtgagggagctcctggtgctgaaggatcccctggacgagatggttctcctggcgccaagggtgaccgtggtgagaccggccctgctggacctcctggtgctcctggcgctcccggtgcccccggccctgtcggacctgccggcaagagcggtgatcgtggtgagaccggtcctgctggtcctgctggtcccattggccccgttggtgcccgtggccccgctggaccccaaggcccccgtggtgacaagggtgagacaggcgaacagggcgacagaggcattaagggtcaccgtggcttctctggtctccagggtccccccggccctcccggctctcctggtgagcaaggtccttccggagcctctggtcctgctggtccccgcggtccccctggctctgctggttctcccggcaaagatggactcaatggtctcccaggccccatcggtccccctgggcctcgaggtcgcactggtgatgctggtcctgctggtcctcccggccctcctggaccccctggtcccccaggtcctcccagcggcggctacgacttgagcttcctgccccagccacctcaagagaaggctcacgatggtggccgctactaccgggctgatgatgccaatgtggtccgtgaccgtgacctcgaggtggacaccaccctcaagagcctgagccagcagatcgagaacatccggagccctgaaggcagccgcaagaaccccgcccgcacctgccgtgacctcaagatgtgccactctgactggaagagcggagaatactggattgaccccaaccaaggctgcaacctggatgccattaaggtcttctgcaacatggaaaccggtgagacctgtgtataccccactcagcccagcgtggcccagaagaactggtatatcagcaagaaccccaaggaaaagaggcacgtctggtacggcgagagcatgaccggcggattccagttcgagtatggcggccaggggtccgatcctgccgatgtggccatccagctgactttcctgcgcctgatgtccaccgaggcctcccagaacatcacctaccactgcaagaacagcgtggcctacatggaccagcagactggcaacctcaagaaggccctgctcctccagggctccaacgagatcgagatccgggccgagggcaacagccgcttcacctacagcgtcacctacgatggctgcacgagtcacaccggagcctggggcaagacagtgatcgaatacaaaaccaccaagacctcccgcttgcccatcatcgatgtggcccccttggacgttggcgccccagaccaggaattcggcttcgacgttggccctgcctgcttcctgggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 62 - protein, 1705 aa, synthetic construct
  REGION 1..1705: note = BtCOL1A1-TEV-GFP-HIS-ePTS1
MQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFLGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 63 - DNA, 4080 bp, synthetic construct
  misc_feature 1..4080: note = BtCOL1A2-TEV-GFP-HIS-ePTS1
  source 1..4080: mol_type = other DNA
atgcaatccttacaagaggcaactgcaagaaagggcccaagtggagatagaggaccacgcggagaaaggggtccaccaggcccaccaggcagagatggtgatgacggcatcccaggccctcctggcccccctggccctcctggcccccctggtcttggcgggaactttgctgctcagtttgatgcaaaaggaggtggccctggaccaatggggctgatgggacctcgcggccctcctggggcttctggagcccctggccctcaaggtttccagggacctccgggtgagcctggtgaacctggtcagactggtcctgcaggtgctcgtggcccgcctggccctcctggcaaggctggtgaggatggtcaccctggaaaacctggacgacctggtgagagaggggttgttggaccacagggtgctcgtggctttcctggaactcctggactccctggcttcaagggcattaggggtcacaatggtctggatggattgaagggacagcctggtgctccaggtgtgaagggtgaacctggtgcccctggtgaaaatggaactccaggtcaaacgggagcccgtggtcttcctggtgagagaggacgtgttggtgcccctggcccagctggtgcccgtggaagtgatggaagtgtgggtcctgtgggccctgctggtcccattgggtctgctggccctccaggcttcccaggtgctcctggccccaagggtgaactcggacctgttggtaaccctggccctgctggtcccgcgggtccccgtggtgaagtgggtctcccaggcctttctggccctgtcggacctcctggaaaccccggagccaatgggcttcctggcgctaagggtgctgctggccttcccggtgttgctggggctcccggcctccctggaccccggggtattcctggccctgttggcgctgctggtgctactggcgccagaggacttgttggtgagcccggcccagctggttcgaaaggagagagcggcaacaagggcgagcctggtgctgttgggcagccaggtcctcctggccccagtggtgaagaaggaaagagaggctccactggagaaatcggacccgctggccccccaggacctcctgggctgaggggaaatcctggctcccgtggtctacctggagctgacggcagagctggtgtcatgggtcctgctggtagccgtggtgcaactggccctgctggtgtgcgaggtcccaatggagattctggtcgccctggagagcctggcctcatgggaccccgaggtttcccaggttcccctggaaatatcggcccagctggtaaagaaggtcctgtgggtctccctggtattgacggcagacctgggcccattggcccagcgggagcaagaggagagcctggcaacattggattccctggacccaaaggccccagtggtgatcctggcaaagctggtgaaaaaggtcatgctggtcttgctggtgctcggggcgctccaggtcccgatggcaacaacggtgctcagggaccccctggactacagggtgtccaaggtggaaaaggtgaacagggtcctgctggtcctccaggcttccagggtctgcctggccctgcaggcacagctggtgaagctggcaaaccaggagaaaggggtatccctggtgaatttggtctccctggccctgctggtgcaagaggggagcgggggcccccaggtgaaagtggtgctgctgggcctactgggcctattggaagccgaggtccttctggacccccagggcctgatggaaacaagggtgaaccgggtgtggttggcgctccaggcactgctggcccatctggtcctagcggactcccaggagagaggggtgcggctggcattcctggaggcaagggagaaaagggtgaaactggtctcagaggtgacattggtagccctggtagagatggtgctcgtggtgctcctggtgctattggtgctcctggccctgctggagccaatggggaccggggtgaagctggtcccgctggccctgctggccctgctggtcctcgtggtagccctggtgaacgtggtgaggtcggtcccgctggccccaacggatttgctggtcctgctggtgctgctggtcaacctggtgctaaaggagagagaggaaccaaaggacccaagggtgaaaatggtcctgttggtcccacaggccccgttggagctgccggtccgtctggtccaaatggcccacctggtcctgctggaagtcgtggtgatggagggccccctggggctactggtttccctggtgctgctggacggactggtccccctggaccctctggtatctctggcccccctggcccccctggtcctgctggtaaagaagggcttcgtgggcctcgtggtgaccaaggtccagttggtcgaagtggagagacaggtgcctctggccctcctggctttgttggtgagaagggtccctctggagagcctggtactgctgggcctcctggaaccccaggtccacaaggccttcttggtgctcctggttttctgggtctcccaggctctagaggtgagcgtggtctaccaggtgtcgctggatctgtgggtgaacctggccccctcggcatcgcaggcccacctggggcccgtggtccccctggtaatgtcggtaatcctggcgtcaatggtgctcctggtgaagccggtcgtgacggcaaccctgggaatgacggtcccccaggccgcgatggtcaacccggacacaagggggagcgtggttaccccggtaacgcaggtcctgttggtgctgccggtgctcctggccctcaaggccctgtgggtcccgttggtaaacacggaaaccgtggtgaaccgggtcctgccggtgctgttggtcctgctggtgccgttggcccaagaggtcccagtggcccacaaggtattcgaggtgacaagggagagcctggtgataagggtcccagaggtcttcctggcttaaagggacacaatgggttgcaaggtctcccgggtcttgctggtcatcatggcgatcaaggtgctcccggtgctgtgggtcccgctggtcccaggggccctgctggtccttctggccccgctggcaaagacggtcgcattggacagcctggtgcagtcggacctgctggcattcgtggctctcagggtagccaaggtcctgctggccctcctggtccccctggccctcctggacctcctggcccaagtggtggtggttacgagtttggttttgatggagacttctacagggctggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 64 - protein, 1359 aa, synthetic construct
  REGION 1..1359: note = BtCOL1A2-TEV-GFP-HIS-ePTS1
MQSLQEATARKGPSGDRGPRGERGPPGPPGRDGDDGIPGPPGPPGPPGPPGLGGNFAAQFDAKGGGPGPMGLMGPRGPPGASGAPGPQGFQGPPGEPGEPGQTGPAGARGPPGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGELGPVGNPGPAGPAGPRGEVGLPGLSGPVGPPGNPGANGLPGAKGAAGLPGVAGAPGLPGPRGIPGPVGAAGATGARGLVGEPGPAGSKGESGNKGEPGAVGQPGPPGPSGEEGKRGSTGEIGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAGARGERGPPGESGAAGPTGPIGSRGPSGPPGPDGNKGEPGVVGAPGTAGPSGPSGLPGERGAAGIPGGKGEKGETGLRGDIGSPGRDGARGAPGAIGAPGPAGANGDRGEAGPAGPAGPAGPRGSPGERGEVGPAGPNGFAGPAGAAGQPGAKGERGTKGPKGENGPVGPTGPVGAAGPSGPNGPPGPAGSRGDGGPPGATGFPGAAGRTGPPGPSGISGPPGPPGPAGKEGLRGPRGDQGPVGRSGETGASGPPGFVGEKGPSGEPGTAGPPGTPGPQGLLGAPGFLGLPGSRGERGLPGVAGSVGEPGPLGIAGPPGARGPPGNVGNPGVNGAPGEAGRDGNPGNDGPPGRDGQPGHKGERGYPGNAGPVGAAGAPGPQGPVGPVGKHGNRGEPGPAGAVGPAGAVGPRGPSGPQGIRGDKGEPGDKGPRGLPGLKGHNGLQGLPGLAGHHGDQGAPGAVGPAGPRGPAGPSGPAGKDGRIGQPGAVGPAGIRGSQGSQGPAGPPGPPGPPGPPGPSGGGYEFGFDGDFYRAGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 65 - DNA, 1092 bp, synthetic construct
  misc_feature 1..1092: note = BtCOL1A1 403-0P-TEV-GFP-HIS-ePTS1
  source 1..1092: mol_type = other DNA
atgggatttgctggccctaaaggagccgcaggagaggctggtaaagcaggcgaacgtggcgtagctggcccagctggtgctgtgggtccagttggtaaagatggagaggccggtgcccaaggtccagccggtcctgtaggcccagcaggtgaaagaggagaacaaggtcccgcaggctcagctggattccaaggattagcaggacctgccggcccagttggcgaagccggtaaagccggagagcaaggcgtggctggtgacctaggtgcagttggccctagtggcgccaggggagaaagaggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 66 - protein, 363 aa, synthetic construct
  REGION 1..363: note = BtCOL1A1 403-0P-TEV-GFP-HIS-ePTS1
MGFAGPKGAAGEAGKAGERGVAGPAGAVGPVGKDGEAGAQGPAGPVGPAGERGEQGPAGSAGFQGLAGPAGPVGEAGKAGEQGVAGDLGAVGPSGARGERGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 67 - DNA, 1092 bp, synthetic construct
  misc_feature 1..1092: note = BtCOL1A1 403-11P-TEV-GFP-HIS-ePTS1
  source 1..1092: mol_type = other DNA
atgggatttcctggccctaagggagccgcaggagagcccggtaaagcaggcgaaagaggcgtacctggtccacccggtgctgtgggtccagctggtaaagatggagaggccggtgcccaaggtcctcctggtcctgctggcccagcaggtgaaagaggagaacaaggtcccgcaggctcacctggattccaaggattaccaggtccagccggaccacctggcgaagccggtaaacccggagagcaaggcgtgcctggtgacctaggtgcaccaggacctagtggcgccaggggagaaagaggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaa

SEQ ID NO: 68 - protein, 363 aa, synthetic construct
  REGION 1..363: note = BtCOL1A1 403-11P-TEV-GFP-HIS-ePTS1
MGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGSENLYFQGRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHHLGRGRRSKL

SEQ ID NO: 69 - DNA, 81 bp, synthetic construct
  misc_feature 1..81: note = T4 fibritin foldon domain
  source 1..81: mol_type = other DNA
ggttacatccccgaagctcctcgtgacggccaggcttacgtcaggaaagatggcgagtgggttcttttgtccacttttctg

SEQ ID NO: 70 - protein, 27 aa, synthetic construct
  REGION 1..27: note = T4 fibritin foldon domain
GYIPEAPRDGQAYVRKDGEWVLLSTFL

SEQ ID NO: 71 - protein, 99 aa, synthetic construct
  REGION 1..99: note = Collagen substrate 1
GFAGPKGAAGEAGKAGERGVAGPAGAVGPVGKDGEAGAQGPAGPVGPAGERGEQGPAGSAGFQGLAGPAGPVGEAGKAGEQGVAGDLGAVGPSGARGER

SEQ ID NO: 72 - protein, 99 aa, synthetic construct
  REGION 1..99: note = Collagen substrate 2
GFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGER

SEQ ID NO: 73 - DNA, 6481 bp, synthetic construct
  misc_feature 1..6481: note = OgPDI Construct
  source 1..6481: mol_type = other DNA
ttgcttgtgtgcagttgcggtgtttttcgggggaggtttcgtttattcgcttttcaaatataaccagggccgtcaatcagtttctcactggaaacgcaattccttcacagctggagtgaaaagccttgctcaggtaagtcaacgtgaagagatttacaaataacgagatactaatatcgactcaatcggctatagagggaatttatgcacaatcgataccgtgataatgtgccagaattcaatagctttttcagctcattttcaggagatgaaaacgagttgattgatgcactggacgcgcaacagaatgaaaactaatagaccatttagcagtctatatcaaaggtgaacaattaagttacatcttattacccggcattaacgttccgggtaaccaaacttataaggccgaaaatttttgcgttgaggcgaactgaataaggtgggaattgacttgaaaaattttcttcagcaggtttttcaaagactgaatactataaatatgatatgcaatggtcaatggaccagtgtcggctcagccggcttgtactacaccatcaaggctgatagtatgtgcgtagacatacactatacggacggcttcattcaaccttcctgtcagggcctacaggttattggaccatgcaaccgttatcagaacggccctagggattttgtagcctgccagacatctggaggtagtggccaccccatctgtattcagtccaccaacggcaacattgagctgtgcgctaactgttactgtcctcaaggttctgagaatctttattttcagggccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggttctcatcatcatcatcatcacttgggaagaggtagaagatccaaattgtaactcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatgctgacggggtcatcacggctcatcatgcgccaaacaaatgtgtgcaatacacgctcggatgactgcatgatgaccgcactgactggggacagcagatccacctaagcctgtgagagaagcagacacccgacagatcaaggcagttaaacgccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaaagatctatggtgtccaaaggagaggagttaatcaaggaaaacatgagaatgaaagttgtcatggagggctccgttaatggtcaccaattcaagtgtacaggggaaggtgaaggtaatccttacatgggtacacaaactatgagaattaaagtaattgaaggcggaccactaccatttgcatttgacattctggcaacgtcattcatgtacggatcacgaactttcatcaagtaccctaaaggtataccagactttttcaagcaatcttttccagagggttttacatgggaaagggttacaagatacgaagatgggggtgtcgtcacagttatgcaagatacttcattagaagatggctgccttgtctatcatgtgcaagtaagaggggtgaattttccttctaacggacctgtgatgcagaaaaagaccaaaggttgggaaccaaatactgaaatgatgtacccagctgatggaggtttgagaggctacacacacatggcgcttaaagttgatggtggaggtcatttgtcttgtagttttgttaccacttatcgttctaaaaagactgttggcaatatcaaaatgccaggaatacatgctgtagaccacagactagaaagactcgaagagagcgataacgaaatgttcgttgtacagagagagcatgccgtagccaaatttgctggcttaggcggtggtatggatgaattgtataagggttctgctgtggccaaaggtgacgccgacgaagccgccattgcgtcgccggattccgctgttgtgaagttgacagcagaatccttcgagtcgtttatcaaggagaacccgctcgttttggctgagttctttgcgccatggtgtggccactgcaagcgtcttggaccagagtttagcgctgctgccgacaaacttgtcgagaaagacatcaagttggcccagattgattgcacccaagagagagatctatgtgcggactatggtatccgtggttatccatctctcaaggtcttcagaggcaacaacacgccatccgagtaccagggccaaagagaacaagatgcaattgtcagctacatgatcaagcaagccctacctccagtgtcgttgcttgaggatacggctgatctgctggacgctctggccgatctgagcgaaccaatgatcttgcaagttctgccacctgactcgaagtcttccggcaacgaaacgttccattcgttggccaaccgtcttagaaacgacttcaggttcgtgtctacctccaaccctgagtatgttgagaaatacgtcaaggaaaagtccactccaacctacgttgttttcagaccgggtgaaaagattgaggacgcatctgttctcaccaacaagactatagacgaagagggattgcagagattcattagtgttgagactaagcctcttttcggcgaggtcaccggtgccacgttccaggcctacatggactccaaacttcctttagcatactttttctatgaagaggagtctcagaaggctgctgtcgcagacgaaatcactaagttggccaagaaatatagaggcgagatcaatttcgccggactggaggccaagaaatacggaatgcacgctaaaaacctcaacatgcaagaaaagttcccactgtttgccattcacgatctgcaaggcgacctaaagtacggcatcccacaagataaggatctggacttctctgagattcctaaatttgtcgaaaacttcaagaagggcaagctgaagccaattgtcaagagcgagcctattccagagactcaagaggaggctgtctaccacttggtcggctacgagcacgacaagatcgtcaaccaaaagaaggacgttctggtcgagtactatgctccatggtgtggtcactgcaagagacttgcccctacatatgaggagctggctgctatctacaagaacgacaccgctgctagtgccaaggtcgtgatcgccaagattgaccacaccgctaacgatgttgcgggcgtcgagatcaccggataccctaccattttcctttatccagctgacggttctggtccggtcaattacgagggacaaagaactttggagtccctagcttctttcattcaagagaagggtacctttggtgttgacggtttggccatcagaggcgctaagagcggcggagctgataaaccggagtccgacactaaggacagtactggatccttgggaagaggtagaagatccaaattgtaactcgagtggcgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcgctggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccacgtctcaagcagttacagagatgttacgaaccactagtgcactgcagtacagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctccagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgggtactaccttagatgatacagcctacagatacagaacatcagtccctggtgatgctgaagcaattgaggctttagacggttcattcaccaccgacaccgtctttagagtaaccgccaccggtgatggatttaccttaagagaagtcccagtcgaccctccattaactaaagtctttccagatgatgaatctgatgacgaaagcgacgacggagaagatggtgacccagattcaagaactttcgtagcatacggtgatgacggtgatttggctggttttgtagtcgtttcttattcaggttggaatagaaggttgaccgttgaagatatagaagtcgccccagagcatagaggtcatggtgtaggaagagctttgatgggtttggctacagaatttgcaagagagagaggagccggtcatttatggttagaagttactaatgttaacgcccctgctatccatgcttatagaagaatgggtttcacattatgtggtttagatactgctttatatgatggaacagcatctgacggtgaacaggccttgtatatgtctatgccttgcccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactg

SEQ ID NO: 74 - protein, 54 aa, synthetic construct
  REGION 1..54: note = Human insulin precursor
MFVNQHLCGSHLVEALYLVCGERGFFYTPKAAKGIVEQCCTSICSLYQFENYCN

SEQ ID NO: 75 - protein, 166 aa, synthetic construct
  REGION 1..166: note = Alpha interferon
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 76 - protein, 89 aa, synthetic construct
  REGION 1..89: note = Mapacalcine
ICNGQWTSVGSAGLYYTIKADSMCVDIHYTDGFIQPSCQGLQVIGPCNRYQNGPRDFVACQTSGGSGHPICIQSTNGNIELCANCYCPQ

SEQ ID NO: 77 - protein, 496 aa, synthetic construct
  REGION 1..496: note = OgPDI
MAVAKGDADEAAIASPDSAVVKLTAESFESFIKENPLVLAEFFAPWCGHCKRLGPEFSAAADKLVEKDIKLAQIDCTQERDLCADYGIRGYPSLKVFRGNNTPSEYQGQREQDAIVSYMIKQALPPVSLLEDTADLLDALADLSEPMILQVLPPDSKSSGNETFHSLANRLRNDFRFVSTSNPEYVEKYVKEKSTPTYVVFRPGEKIEDASVLTNKTIDEEGLQRFISVETKPLFGEVTGATFQAYMDSKLPLAYFFYEEESQKAAVADEITKLAKKYRGEINFAGLEAKKYGMHAKNLNMQEKFPLFAIHDLQGDLKYGIPQDKDLDFSEIPKFVENFKKGKLKPIVKSEPIPETQEEAVYHLVGYEHDKIVNQKKDVLVEYYAPWCGHCKRLAPTYEELAAIYKNDTAASAKVVIAKIDHTANDVAGVEITGYPTIFLYPADGSGPVNYEGQRTLESLASFIQEKGTFGVDGLAIRDAKSGGADKPESDTKDST

SEQ ID NO: 78 - DNA, 675 bp, synthetic construct
  misc_feature 1..675: note = Human beta-Casein II
  source 1..675: mol_type = other DNA
atgaaagtccttattttagcttgccttgtcgcattggctctggcaagagagacgattgaatcactaagtagttccgaagaaagtatcaccgaatataaaaaggtcgagaaggtgaagcatgaagaccagcaacagggcgaagacgagcatcaagacaagatttaccctagtttccaaccacagcctttaatttatcccttcgtggaaccaataccatatggcttcctgccacaaaatatcctgcccttagcccaacccgccgtcgttctgccagtgcctcaacctgagatcatggaagttccaaaagccaaggatactgtttatactaaaggacgtgtgatgcctgttttaaaatctcccaccattcctttctttgatccccaaatcccaaaacttactgaccttgagaacctacatctacccctaccacttttacagccactaatgcaacaggtgcctcagcctattcctcagaccctagctctaccaccacagcccctttggtctgtcccccaacccaaggttcttcccatacctcaacaagtagttccatacccacaacgtgctgtccctgtgcaggctctgctactgaaccaggaattgttactgaatcctacccaccaaatctacccagtgactcagcccttagccccagtacataatcccatcagtgtt

SEQ ID NO: 79 - DNA, 645 bp, synthetic construct
  misc_feature 1..645: note = Casein Kinase II
  source 1..645: mol_type = other DNA
atgtcatcctccgaggaagtcagttggatctcatggttctgcggtctgaggggcaacgagtttttctgtgaggtagatgaagactatattcaagacaagttcaatctgacgggacttaatgaacaggttcctcactacagacaagcactagacatgatattagacctggagcctgacgaagaactagaagataaccccaatcagtcagatctaatcgaacaagccgcagagatgttgtatggcttgatacacgccagatacatattaactaaccgtggtattgcacagatgttggaaaagtatcagcaaggtgattttggatattgccccagagtatattgcgagaaccaacctatgttacccataggactttctgatattcctggagaggctatggtgaaattgtactgccctaaatgtatggatgtttacactcctaaatcttcccgtcatcatcatacggatggcgcttattttggaactggttttccccacatgttgttcatggtccaccctgagtataggccaaaaagacctgcaaatcaatttgttcctagactttatggatttaagatacatccaatggcttaccaactgcagttacaagctgctagtaattttaaatctccagtcaaaaccattaga

SEQ ID NO: 80 - DNA, 1155 bp, synthetic construct
  misc_feature 1..1155: note = Ovalbumin
  source 1..1155: mol_type = other DNA
ggttctatcggagcagctagtatggaattctgtttcgacgtgttcaaggaattaaaagtccatcatgctaatgaaaacatattctactgcccaattgccattatgtcagccctggccatggtgtacctaggtgccaaagattccacgagaactcaaataaataaggttgttagattcgacaagttgccaggtttcggtgatagtattgaagcccagtgcggaacgtctgttaacgttcacagttccctaagagacattttaaatcaaatcacaaagcccaacgacgtgtattcattttccttagcctccaggctgtacgccgaggaaagatatccaattttgcccgaatacctgcagtgcgtaaaagagctgtatagaggcggacttgaaccaataaatttccagaccgctgctgaccaagcccgtgagttgataaactcctgggtcgagtcccaaactaatggaatcatacgtaacgtgctgcaaccaagtagtgttgactcacagaccgcaatggttttggttaacgctatcgtatttaaaggtttgtgggagaaagcattcaaagacgaagacacacaagccatgcctttcagagtgacggagcaggagagtaagccagtacagatgatgtaccagattggccttttcagagttgcttccatggcctcagagaagatgaaaatcctggagcttccatttgcttctggtactatgtccatgctagtgcttctgcccgacgaagtgtctggattggagcaattagaatctatcataaactttgaaaagttaacagagtggacttcttcaaacgtaatggaggaacgtaagattaaagtttacctaccacgtatgaaaatggaagaaaagtataatttgacctctgttctgatggcaatgggcataaccgacgtattcagttcttcagcaaatttatccggcatatcctctgcagaatctctaaagatatctcaggccgtacacgccgctcacgcagaaattaacgaggccggacgtgaggtagtaggatccgctgaggccggtgtggacgctgcatctgtgtctgaggagtttagagccgaccacccttttctattctgtattaaacatatagccactaatgccgttcttttcttcggtaggtgcgtctcacca

SEQ ID NO: 81 - DNA, 540 bp, synthetic construct
  misc_feature 1..540: note = NatB complex Naa20
  source 1..540: mol_type = other DNA
atgaccgatacaagaaaatttaaggcaacagatttattttcctttaacaacataaatctggacccacttaccgaaacatttaacatatcattttacctttcatatcttaacaagtggccctccttatgcgtagtacaagagtccgatctttcagaccccacgttgatgggttatattatgggcaagtccgaaggaacaggcaaagagtggcacacgcatgttaccgccatcacagtggccccaaattcacgtagattgggccttgccaggacaatgatggattacttggagacggtcggtaactctgagaacgccttcttcgtggacctgttcgtcagggcatccaacgccctagcaattgacttttacaaaggattgggctactctgtctaccgtagagtgattggttattacagtaaccctcatggcaaagatgaagactctttcgacatgagaaaaccattgtctagggatgtaaacagggagtcaatcagagaaaacggtgagaactttaagtgctcacccgcagatgtgagtttt

SEQ ID NO: 82 - DNA, 2433 bp, synthetic construct
  misc_feature 1..2433: note = NatB complex Naa25
  source 1..2433: mol_type = other DNA
atgaggaggagtggctccaaggagagtactattgtatactcagctttgagtcttgctcaggccggaagaggacccgaagcactggccttgcttgagcccctgaagtcaactccaatcaattctcttgagttattggatatcatacaggctgtatacgatgatcaaaagaaaggagaagaatccttcgttttctgggagaagttccttcagacttatggtaagcaggagaaaaatttactggcttacttcaaggcttctattagaattaaatctctgagtcaccaacgtaaggctgcagtggagctgcaaaagaactttccaagtaggaaacacacgttatgggttattagtagtctttatttactgtccaaaaaatccgagaacgaagttgagcagaggctgctgaaggctctagctgaaaaaaccgctaaattaatttttgaaaaaccaaccggatatattgattcttgtgaggaattccatttgtatctggacgtactacttctagtaggtgataaagatagggctcttgacgctttaattcaccaagacgcagatagattcgtcgacgctgatgctgacctactgcttcgtaaattagaactattagcaagttgtgcaagatgggattcactgtttactttctccctgagtttgttccagactggtaacacggactggaaagtctgtaaagcactactagattctgcctccaacgatgatagtaagttagtaccattgaaggattgcatacttaaagcattatctacgtcttctactaaaagaaatttacacctgctatggattgaagcatccgcacgtttctttcccgaggaacacgagtcagcactattaggctacataaagaaactttacatgaagcccattgtcttcgaggatcttaggccatatctgctgaaactaaacgtcgatgcacagcaccgtttgttggacgctttcaagctagctgatttgggcgagtcaaatgagtcacagaaggtcgataaattatacgctgaggttcttctgttgaaaatccacttcctgctattcgagagtttcacagccgagagtgtggtagactacgttcgtcgttgtttcgttgcctttgagaaaggactttcactgtctaaaggacttttgccaacggacttcactcatggatatgaggctcttctgttagcagtacattcccttatttatatgtgggaaggtaacaaggatttaaaaccagcagaaaagcaggcattaattttcgacgctatttgcctgttagaaaagggtataacatacagtcaacataatttccacctgaaacttcccctgataaggttatacctactgcttgacggaggattccccgcagcagcaaaagtttacgatactatgagtataaagcaaattcagaacgatacattagatcactatttactgaccagggctaccacatattacccctcttccgtcacgtcacattatataaattcatccttaaagatatatggctctaacgagttcgaaaccccagaaatgatttctatggcatatgaggacggcgcatacagtcagatcgaggacatgcgtaattttagatctaggcttgaccattccacctggaagagtatatccctagtcgaaagggccaggatacactatcttaccgcatttaagcctcctaaacagtacctacccaagtgttccagtcctaaagataaccgtgacctaaaggtgttcgctgattacggatcagacaagcttcctaccgtggaggaaagtctaaggaactcccccaagcccgatacgttgtggatccacctaactgtaatcggtcattccttagttcaggatagtattgtgaatggcgattttgagaaggccgttctgtcagccaaagaaatggaagtcttgtgtgaaaataacgatctgtctaagcaactaacatcagaagagatcgtgcacatgaagctactaatccaattaggacttttaagtgtgaaggttaagaatggagattatgaaaactcctcttttgagactatcgagaaccttatagaaagtttcgattatgaaaacagtactcccctaagtcagttgacaaagtataccgagatcatcaatgatttaatcacctgcttgaactcatttttgtatcatgtaagtgctactaagaaaaaggaattcacacgtcaataccagttgctgaaaaatataagttccaacaaacttggatcaatctctggtatcaccaaacataagaagaaagctgccagaaagtacgtttccgagctgttgagtaattcttggctaagtaacctatctgagacccaggtcccttacgatccaaagtttgcaaagcaagtgggtgagggtatgatcgactcttatatacagacaacggacgcagtgtcaaaattaccaaagttcgtgaagttt

(19) United States
(12) Patent Application Publication
(10) Pub. No.: US 2026/0234647 A1
(43) Pub. Date: Aug. 13, 2026

(54) EXPRESSION OF MODIFIED PROTEINS IN A PEROXISOME

(71) Applicant: Provenance Bio, LLC, San Francisco, CA (US)

(72) Inventors: Genevieve M. Vidanes, San Francisco, CA (US); John Dueber, San Francisco, CA (US); Zev Gartner, Pacifica, CA (US); Chirag Vazirani, Berkeley, CA (US)

(73) Assignee: The Regents of the University of California, Oakland, CA (US)

(21) Appl. No.: 19/260,158

(22) Filed: Jul. 3, 2025

Related U.S. Application Data
(63) Continuation of application No. 17/595,293, filed on May 12, 2020, now abandoned.
(60) Provisional application No. 62/847,769, filed on May 14, 2019.

(51) Int. Cl. C12N 15/81; C07K 14/47; C07K 14/65; C07K 14/77; C07K 14/78; C12N 9/02; C12N 9/12; C12N 9/50; C12N 9/90; C12N 15/52; C12N 15/62

(52) U.S. Cl. CPC C12N 15/81; C07K 14/4732; C07K 14/65; C07K 14/77; C07K 14/78; C12N 9/0071; C12N 9/12; C12N 9/506; C12N 9/90; C12N 15/52; C12N 15/62; C12Y 114/11002; C12Y 207/11001; C12Y 304/22044; C12Y 503/04001; C07K 2319/50; C12N 2800/22

(57) ABSTRACT

Disclosed herein include methods and compositions for making proteins in peroxisomes as well as methods of making cells for producing proteins in peroxisomes. Also disclosed herein are cells for producing a protein in a peroxisome, and methods for producing a protein in a eukaryotic cell containing a peroxisome as described herein.

### CROSS REFERENCE TO RELATED APPLICATION

This application is a continuation application of the U.S. application Ser. No. 17/595,293, filed on Nov. 12, 2021, which is a continuation of International Application No. PCT/US2020/032512, filed May 12, 2020, which claims the benefit of U.S. Provisional Application No. 62/847,769, filed May 14, 2019, which is hereby incorporated by reference in its entirety.

### REFERENCE TO SEQUENCE LISTING AND TABLES IN ELECTRONIC FORMAT

This application is filed with an electronic sequence listing entitled PBFAB.001C1SEQLISTING.xml, created on Jul. 3, 2025 which is 182,986 bytes in size. The information in the electronic sequence listing is hereby incorporated by reference in its entirety.

### FIELD

Methods and compositions are provided herein for genetically modifying cells to produce proteins and protein precursors that for example may be used in artificial materials.

### BACKGROUND

There is a need in the art for improved methods of producing and modifying proteins in cells. Proteins produced and modified in cells find use in a variety of ways.

### SUMMARY

Described herein are methods for producing proteins that can act as precursors for materials, such as substrates for products in film development; capsules for pills (gelatin in drug and nutraceuticals); food additives (e.g. all things gelatin) and collagen for food stuffs and synthetic meats, textiles such as synthetic leather, beauty products, and biomedical materials (scaffolds, sutures, grafts, expanding cells, gels, etc.) are contemplated. The use of such methods may also provide materials that would reduce the product carbon footprint from standard manufacturing methods that are used today.

Protein precursors that may be used in the production of materials are contemplated. For example, a next generation fabric is contemplated, such as artificially made textiles, using cell engineering and tissue engineering techniques that lower greenhouse gas emissions as compared to conventionally produced textiles.

The protein precursors may be used as collagen derived products that can be found in face creams, injectable drugs and wound dressings, for example.

Methods and compositions are provided herein for genetically modifying cells to produce proteins and protein precursors, for example those can be used in artificial materials.

Some embodiments provided herein relate to methods and compositions for making genetically modified cells to produce modified proteins in peroxisomes. Modified proteins described herein may be used as building blocks for producing materials, such as textiles, artificial skins or other materials. Production of proteins found in some textiles are contemplated for use in a cell production system.

Some embodiments provided herein relate to methods of making a cell for producing a modified protein in a peroxisome. In some embodiments, the methods include the steps: providing a cell, introducing a first nucleic acid into the cell and introducing a second nucleic acid into the cell. In some embodiments, the first nucleic acid includes a first sequence encoding a heterologous protein fused to a peroxisome-targeting sequence. In some embodiments, the second nucleic acid includes a second sequence encoding heterologous modification enzyme fused to a peroxisome-targeting sequence. In some embodiments, the cell is a bacterial or archaebacteria. In some embodiments, the cell is a eukaryotic cell. In some embodiments, the cell is a yeast cell. In some embodiments, the cell is a yeast cell. In some embodiments, the cell is selected from the genera Arxula, Candida, Hansenula, Kluyveromyces, Komagataella, Ogataea, Pichia, Saccharomyces or Yarrowia. In some embodiments, the first and/or second nucleic acid includes a promoter(s). In some embodiments, the promoter is constitutive or inducible. In some embodiments, the peroxisome-targeting sequence includes a sequence set forth in SEQ ID NO: 1 (SLK), SEQ ID NO: 2 (RLXXXXX(H/Q)L), or SEQ ID NO: 3 (LGRGRRSKL). In some embodiments, the protein includes a tag. In some embodiments, the tag is cleavable. In some embodiments, the method further includes introducing a third nucleic acid into the cell. In some embodiments, the third nucleic acid includes a third sequence encoding a second heterologous modification enzyme fused to a peroxisome-targeting sequence. In some embodiments, the heterologous protein has a molecular weight of 1 Da, 5 Da, 10 Da, 20 Da, 30 Da, 40 Da, 50 Da, 60 Da, 70 Da, 80 Da, 90 Da, 100 Da, 200 Da, 300 Da, 400 Da, 500 Da, 600 Da, 700 Da, 800 Da, 900 Da, 1 kDa, 5 kDa, 10 kDa, 20 kDa, 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, or 300 kDa, or any size in between a range defined by any two aforementioned values. In some embodiments, the enzyme creates a modification. In some embodiments, the modification is folding of the protein. In some embodiments, the protein is unfolded. In some embodiments, the modification is protein folding, hydroxylation, glycosyl transfer, oxidation, and/or isomerization. In some embodiments, the enzyme includes prolyl hydroxylases, glycosyltransferase, lysyl oxidases, a protein chaperone, or prolyl isomerase. In some embodiments, the enzyme is a glycosyltransferase, prolyl isomerase, a protein disulfide isomerase, a hydroxyl transferase, or a prolyl hydroxylase. In some embodiments, the protein includes collagen, gelatin, or silk protein. In some embodiments, the enzyme includes glycosyl transferase, prolyl hydroxylase, or prolyl isomerase. In some embodiments, wherein the protein is collagen, the collagen is modified resulting in a Type I heterotrimer, Type 1 alpha homotrimer, or Type III homotrimer collagen. In some embodiments, the collagen includes Col1A1 or Col1A2. In some embodiments, the prolyl-4-hydroxylase is genetically modified to have a deletion of a PDI domain. In some embodiments, the enzymes are genetically modified for improved expression and import into the peroxisome. In some embodiments, the proteins are genetically modified for improved expression and import into the peroxisome. In some embodiments, the nucleic acid is codon optimized for protein expression in a eukaryotic cell, such as a yeast cell. In some embodiments, fusion of the heterologous protein to the peroxisome targeting sequence results in targeting of the heterologous protein to the peroxisome, thereby separating the heterologous protein from an enzyme not targeted to the peroxisome. In some embodiments, fusion of the modification enzyme to the peroxisome targeting sequence results in targeting of the modification enzyme to the peroxisome, thereby separating the modification enzyme from a substrate or enzyme not targeted to the peroxisome. In some embodiments, the heterologous protein includes COLsyn1, COLsyn2, COLsyn3, COLsyn4, or an amino acid sequence at least 80%, 85%, 90%, 95%, 97%, 98%, or 99% identical to the amino acid sequence of COLsyn1, COLsyn2, COLsyn3, or COLsyn4. In some embodiments, the first nucleic acid is engineered to replace at least one hydrophobic amino acid with a hydrophilic or non-hydrophobic amino acids in the heterologous protein as compared to an unmodified or naturally occurring first nucleic acid.

Some embodiments provided herein relate to eukaryotic cells for producing a protein in a peroxisome, manufactured by any method provided herein.

Some embodiments provided herein relate to eukaryotic cells for producing a protein in a peroxisome. In some embodiments, the cells include a first nucleic acid including a sequence encoding a heterologous protein fused to a peroxisome-targeting sequence and a second nucleic acid encoding a heterologous modification enzyme fused to a peroxisome-targeting sequence.

Some embodiments provided herein relate to eukaryotic cells that include a peroxisome for producing a modified protein. In some embodiments, the eukaryotic cells are capable of expressing a heterologous protein fused to a peroxisome-targeting sequence, and a heterologous modification enzyme fused to a peroxisome-targeting sequence. In some embodiments, the protein is modified in the peroxisome. In some embodiments, the cell is Pastoris. In some embodiments, the peroxisome-targeting sequence includes a sequence set forth in SEQ ID NO: 1, 2, or 3. In some embodiments, the cell further includes a third nucleic acid encoding a second protein fused to a peroxisome-targeting sequence.

Some embodiments provided herein relate to methods of producing a modified protein in a eukaryotic cell containing a peroxisome. In some embodiments, the eukaryotic cells express a heterologous modification enzyme fused to a peroxisome-targeting sequence. In some embodiments, the methods include: providing a cell manufactured by the method or a cell of any one of the alternatives described herein, expressing a heterologous protein in the eukaryotic cell and culturing the eukaryotic cell under conditions such that the heterologous modification enzyme modifies the heterologous protein in the peroxisome to produce a modified protein. In some embodiments, the heterologous protein is fused to a peroxisome-targeting sequence. In some embodiments, the method further includes increasing cargo of the peroxisome. In some embodiments, increasing cargo of the peroxisome is performed by providing oleic acid or methanol to the eukaryotic cell.

Some embodiments provided herein relate to methods of producing a modified protein in a eukaryotic cell containing a peroxisome. In some embodiments, the eukaryotic cells express a heterologous modification enzyme fused to a peroxisome-targeting sequence. In some embodiments, the methods include expressing a heterologous protein in a eukaryotic cell and culturing the eukaryotic cell under conditions such that the heterologous modification enzyme modifies the heterologous protein in a peroxisome to produce a modified protein. In some embodiments, the heterologous protein is fused to a peroxisome-targeting sequence. In some embodiments, the methods further include increasing cargo of the peroxisome. In some embodiments, increasing cargo of the peroxisome is performed by providing oleic acid or methanol to the eukaryotic cell.

Some embodiments provided herein relate to methods of producing a modified protein. In some embodiments, the methods include culturing a eukaryotic cell containing a peroxisome under conditions such that the modified protein is produced. In some embodiments, the eukaryotic cell expresses: a heterologous protein fused to a peroxisome-targeting sequence, and a heterologous modification enzyme fused to a peroxisome-targeting sequence. In some embodiments, the heterologous modification enzyme modifies the heterologous protein to produce the modified protein in the peroxisome under the culture conditions. In some embodiments, the methods further include increasing cargo of the peroxisome. In some embodiments, increasing cargo of the peroxisome is performed by providing oleic acid or methanol to the eukaryotic cell.

Some embodiments provided herein relate to methods of increasing yield of a modified protein. In some embodiments, the methods include culturing a eukaryotic cell containing a peroxisome under conditions such that the modified protein is produced. In some embodiments, the eukaryotic cell expresses a heterologous protein fused to a peroxisome-targeting sequence and a heterologous modification enzyme fused to a peroxisome-targeting sequence. In some embodiments, expression of the heterologous protein is under the influence of a promoter. In some embodiments, the heterologous modification enzyme modifies the heterologous protein to produce the modified protein in the peroxisome under the culture conditions and inducing production of the heterologous protein by addition of a chemical inducer. In some embodiments, the methods further include increasing cargo of the peroxisome. In some embodiments, increasing cargo of the peroxisome is performed by providing oleic acid or methanol to the eukaryotic cell.

Some embodiments relate to kits for producing a modified protein in a peroxisome in a cell. In some embodiments, the kits include: a first nucleic acid construct including GFP-x-ePTS1 or x-FLAG-ePTS1 and a second nucleic acid construct including GFP-y-ePTS1 or y-FLAG-ePTS1. In some embodiments, x is a nucleic acid sequence encoding a heterologous protein to be targeted to a peroxisome. In some embodiments, y is a nucleic acid sequence encoding a modification enzyme to be targeted to the peroxisome. In some embodiments, the modification enzyme is an enzyme capable of modifying the heterologous protein in the peroxisome.

### BRIEF DESCRIPTION OF THE DRAWINGS

FIG. 1 shows a schematic representing an example of directing a protein and an enzyme into the peroxisome of the cell.

FIG. 2 shows a schematic of the fermentation of the genetically modified yeast, purification of the translationally modified proteins in accordance with some embodiments.

FIG. 3 depicts images of microscopy data of S. cerevisiae strains that are wild type (top row) or modified with deleted PEX5 gene (bottom row) and expressing fusion proteins. The fusions include N-terminal GFP and C-terminal ePTS1 fused to synthetic collagen peptides and a collagen modifying enzyme.

FIG. 4 shows fluorescence localization of collagen variants fused to GFP and a C-terminal ePTS1 in strains PB000095, PB000163, PB000297 that are representative of different industrial yeast hosts, PBH001, PBH002, and PBH004, respectively.

FIG. 5 shows colony growth of strains that have been serially diluted on YPD or YP galactose plates. Strains express GAL-SigD1-351-ePTS1 (top) or GAL-SigD1-351 (bottom).

FIG. 6 shows an image of a Western blot of peroxisome-localized TEV-FLAG-ePTS1 protease activity on peroxisome-localized RFP-tev-TFP-ePTS1 substrate (panel A) or on cytoplasmic RFP-tev-YFP substrate (panel B). The TEV protease expression was controlled by different constitutive or inducible promoters and growth conditions: (1) pTEF1, (2) pRPL18B, (3) pGAL1, repressed by dextrose, (4) pGAL1, repressed by raffinose and dextrose, and (5) pGAL1, induced by raffinose and galactose. Western blots were probed with an anti-tRFP antibody to recognize the full length 54 kDa substrate or 27 kDa cleavage product.

FIG. 7 shows Bant P4H hydroxylase activity on collagen in the peroxisome. Panel A depicts list of strains. The Bant P4H is expressed from the TDH3 promoter and the collagen substrate from the TEF1 promoter. Panel B shows alignment of collagen substrate from each of the strains with Geneious software. The consensus sequence shows that 1. PB000224; 2. PB000248; and 3. PB000249 exhibit the same sequence (SEQ ID NO: 71), and 4. PB000225; 5. PB000254; and 6. PB000255 exhibit the same sequence (SEQ ID NO: 72). The gray boxes below an amino acid denote the proline positions identified to be oxidized by LCMSMS. Panel C shows details of the LCMSMS results at each modified site.

FIG. 8 shows in vivo fluorescence localization of ePTS1-tagged full-length collagen, AmCol1A or AmCol1A2, fused to a GFP tag and ePTS1-tagged BantP4H hydroxylase enzyme fused to an mRuby tag in S. cerevisiae. Images are shown as individual FITC and TexasRed channels for GFP and mRuby detection, respectively. The merged image is an overlap of the FITC and TexasRed channels implying colocalization of both proteins.

### DETAILED DESCRIPTION

### Definitions

The titles, headings and subheadings provided herein should not be interpreted as limiting the various aspects of the disclosure. Accordingly, the terms defined immediately below are more fully defined by reference to the specification in its entirety.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Systeme International de Unites (SI) accepted form. Numeric ranges are inclusive of the numbers defining the range. Measured values are understood to be approximate, taking into account significant digits and the error associated with the measurement.

As utilized in accordance with the present disclosure, the following terms, unless otherwise indicated, shall be understood to have the following meanings:

As used herein, the term “about” refers to a numeric value, including, for example, whole numbers, fractions, and percentages, whether or not explicitly indicated. The term “about” generally refers to a range of numerical values (e.g., +1-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

“Peroxisome” has its plain and ordinary meaning when read in light of the specification, and may include but is not limited to, for example, an organelle for the catabolism of very long chain fatty acids, branched chain fatty acids, D-amino acids, and polyamines, reduction of reactive oxygen species, biosynthesis of plasmalogens, (i.e., ether phospholipids critical for the normal function of mammalian brains and lungs). Peroxisomes may also function for the glyoxylate cycle, glycolysis and methanol and/or amine oxidation and assimilation in some yeasts. Peroxisomes may also have their own natural enzymes. Without being limiting, the enzymes may include, catalases for oxidative enzymes, such as D-amino acid oxidase and uric acid oxidase, for example. In the embodiments herein, the peroxisome may function for making protein or for modification of proteins.

“Modifications” to a protein has its plain and ordinary meaning when read in light of the specification. Without being limiting, modifications may include changes to a protein at the primary, secondary, tertiary, and quaternary structure; addition of a covalent modification, folding of a protein, assembly of proteins into a quaternary structure of a multi-subunit complex, and post-translational modifications. Other modifications in addition to prolyl hydroxylation are also achievable in the peroxisome. The peroxisome is naturally permeable to many small molecules that serve as modifying substrates by the modifying enzymes. In fact, the peroxisome has been determined to have a size gating where molecules smaller than approximately 700 Daltons can freely diffuse into this organelle. Substrates that cannot freely diffuse into the peroxisome must be transported. Transport could be imported, either specifically or promiscuously, via a membrane protein targeted to the peroxisome membrane.

“Nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives, a nucleic acid sequence comprising a sequence encoding a heterologous protein fused to a peroxisome-targeting sequence is provided. In some alternatives, the nucleic acid is RNA or DNA

“Eukaryotic” cells include, but are not limited to, algae cells, fungal cells (such as yeast), plant cells, animal cells, mammalian cells, and human cells (e.g., T-cells). In some embodiments, the cell is selected from a genus of methylotrophic yeasts consisting of Komagataella, Pichia, Hansenula, and Ogataea. In some embodiments, the cell is selected from additional budding yeast genera, Arxula, Candida, Kluveromyces, Saccharomyces and Yarrowia.

“Bacterial cells” has its plain and ordinary meaning when read in light of the specification. Bacterial cells are surrounded by a cell membrane which is made primarily of phospholipids. This membrane encloses the contents of the cell and acts as a barrier to hold nutrients, proteins and other essential components of the cytoplasm within the cell. However, unlike eukaryotic cells, bacteria usually lack large membrane-bound structures in their cytoplasm such as a nucleus, mitochondria, chloroplasts and the other organelles present in eukaryotic cells. Bacteria, for protein expression, may include E. coli, for example.

“Archaebacteria” has its plain and ordinary meaning when read in light of the specification. Archaebacteria or Archaea may live in extreme environments such as at the bottom of the sea by extremely hot hydrothermal vents. Both Archaea and Bacteria are very similar. They both are single-celled prokaryotes that have cell walls and cell-membranes. The main difference between is their chemical structure and where they live. Example may include but are not limited to thermophiles, halophiles, and methanogenes.

A “promoter” has its plain and ordinary meaning when read in light of the specification, and may include, for example, a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SP1, cAMP response element binding protein (CREB; Loeken, Gene Expr. 3:253 (1993); incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety)), and Lemaigre and Rousseau, Biochem. J. 303:1 (1994); incorporated by reference in its entirety). As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. In some embodiments herein, the nucleic acids provided comprise a promoter sequence. In some embodiments, the promoter is a yeast promoter for protein translation. In some embodiments, wherein the cell is Pichia, the promoter comprises methanol inducible promoter, P_{AOX}_{₁} or constitutive promoter P_{GAP}. In some embodiments, the promoter comprises pAOX, pGal, pCup, pGEM, or pZPM.

A peroxisomal targeting signal (PTS) has its plain and ordinary meaning when read in light of the specification, and may include, for example, a region of the peroxisomal protein that receptors recognize and bind to. Proteins containing this motif are localized to the peroxisome. In some embodiments herein, nucleic acids are provided that comprise protein sequences operably linked to a PTS.

A “protein tag” or “tag” has its plain and ordinary meaning when read in light of the specification, and may include, for example, peptide sequences genetically grafted onto a recombinant protein. Often these tags are removable by chemical agents or by enzymatic means, such as proteolysis or intein splicing. Tags are attached to proteins for various purposes, such as, for example, as an affinity tag for purification or solubilization. A tag may also be added to a protein or an enzyme for protein stability while in a peroxisome. In some embodiments herein, the protein expressed for modification in the peroxisome comprises a tag. In some embodiments, the tag is selected from a group consisting of histidine (e.g., HIS6), maltose-binding protein, GST. FLAG, Fc domain, and a Strep-tag.

“Protein” has its plain and ordinary meaning when read in light of the specification, and may include, for example, a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, and selenocysteine. A protein may also comprise unnatural amino acids. In some embodiments, unnatural amino acid incorporation is performed by amber codon suppression. A protein can also comprise non-peptide components, such as carbohydrate groups, for example. Carbohydrates and other non-peptide substituents can be added to a protein by the cell in which the protein is produced, and will vary with the type of cell. Proteins are defined herein in terms of their amino acid backbone structures; substituents such as carbohydrate groups are generally not specified, but can be present nonetheless. In some alternatives described herein, a method of making a modified protein in a peroxisome is provided. In some embodiments, the modified protein comprises collagen, gelatin or a silk protein. In some textiles, proteins such as globulin-like proteins, keratin, collagen hydrolysate, collagen peptides and collagen are also considered.

“Collagen” has its plain and ordinary meaning when read in light of the specification, and may include, for example, a structural protein that is found in skin and other connective tissues. In some embodiments herein, collagen is modified in a peroxisome.

“Gelatin,” has its plain and ordinary meaning when read in light of the specification, and may include, for example, a water-soluble protein prepared from collagen. In some embodiments, gelatin is provided for modification in a peroxisome.

“Isomerases” have their plain and ordinary meaning when read in light of the specification, and may include, for example, an enzyme that catalyzes the conversion of a specified compound to an isomer. Those of skill in the art would understand that there are many types of isomerases, such as, for example, racemases, epimerases, Cis-trans isomerases, and Intramolecular transferases.

“Hydroxyl transferases” have their plain and ordinary meaning when read in light of the specification, and may include, for example, enzymes such as prolyl hydroxylases and lysyl oxidases.

“Glycosyltransferases” have their plain and ordinary meaning when read in light of the specification, and may include, for example, enzymes that establish glycosidic linkages.

Those skilled in the art will appreciate that gene expression levels are dependent on many factors, such as promoter sequences and regulatory elements. Another factor for maximal protein selection is adaptation of codons of the transcript gene to the typical codon usage of a host. As noted for most bacteria and yeast cells, for example, small subsets of codons are recognized by tRNA species leading to translational selection, which can be an important limit on protein expression. In this aspect, many synthetic genes can be designed to increase their protein expression level. The design process of codon optimization can be to alter rare codons to codons known to increase maximum protein expression efficiency. In some alternatives, codon selection is described, wherein codon selection is performed by using algorithms that are known to those skilled in the art to create synthetic genetic transcripts optimized for higher levels of transcription and protein yield. Programs containing algorithms for codon optimization are known to those skilled in the art. Programs can include, for example, OptimumGene™, GeneGPS® algorithms, etc. Additionally, synthetic codon optimized sequences can be obtained commercially for example from Integrated DNA Technologies and other commercially available DNA sequencing services. In some alternatives, proteins are prepared such that the genes for protein for modification are codon optimized for expression in yeast, such as Pichia, for example. In some alternatives, proteins or enzymes are described, wherein the genes for the complete gene transcript for the protein or enzyme are codon optimized for expression in eukaryotic cells, such as yeast, which can increase the concentration of proteins for modification in a yeast peroxisome.

“Purification” has its plain and ordinary meaning when read in light of the specification, and may include, for example, the isolation of highly purified cells, peroxisomes and protein, for example. In a method of cell purification, cells can be isolated, separated, or selected by their capacity to bind to ligand that is attached to a support, such as a plastic or poly carbonate surface, bead, particle, plate, or well. Cells can bind on the basis of particular cell surface markers, which allow them to be purified. In the cases of peroxisome, those of skill in the art would understand the methods for peroxisome purification, such as centrifugation, for example. Proteins can also be purified. Methods of protein purification are known to those of skill in that art, such as, for example, size exclusion, and affinity chromatography.

Textiles and accessories are consumer products that are purchased frequently and replaced most often. Furthermore, most clothing does not last long and requires frequent replacement. For clothing, the high turn-over, large production volumes and energy-intensive use make clothing an important product category in terms of resource consumption and greenhouse gas emissions.

In order to obviate the problems associated with making clothes, several areas will need to be addressed such as the carbon footprint of clothing and accessories. The carbon footprint can be described as a total set of greenhouse gas emissions caused by an organization, event, product or person. As addressed herein are methods and cells to lower the carbon footprint associated with textile production. The carbon footprint of an item of clothing for example, is the total amount of carbon dioxide (CO₂) and other greenhouse gases emitted over the life cycle of that item, expressed as kilograms of CO₂ equivalents. This includes all greenhouse gases generated in the manufacture of the raw materials, fabrication of the item, transport of materials and finished items, packaging, the use phase including numerous washing and drying cycles, and end-of-life disposal.

Protein precursors for other materials are also contemplated. The proteins produced by the cells may be precursors to several materials such as products for film development; capsules for pills (gelatin in drug and nutraceuticals); food additives (e.g. all things gelatin) and collagen for food stuffs and synthetic meats, synthetic leather, beauty products, and biomedical materials (scaffolds, sutures, grafts, expanding cells, gels, etc.) are contemplated.

In order to obviate the problems associated with a high carbon footprint, the methods of making precursors for producing a textile are described. As described in the embodiments herein are methods of making modified proteins in cells within organelles, such as the peroxisome. Peroxisomes are ubiquitous and multifunctional organelles that are primarily known for their role in cellular lipid metabolism. Peroxisomes comprise peroxisomal enzymes that may catalyze redox reactions as part of their normal function, these organelles are also increasingly recognized as potential regulators of oxidative stress-related signaling pathways.

In order for processing to occur within the peroxisome, a protein may be directed by signaling sequence to be translocated to the peroxisome. The sequence encoding the signaling sequence may be operably linked to the sequence encoding the protein. Following translation of the protein, the protein is thus directed to a peroxisome.

Peroxisomes have been well described since their discovery in 1965 (Sabatini et al.; PNAS Aug. 13, 2013. 110 (33) 13234-13235 and Purdue et al.; Annu. Rev. Cell Dev. Biol. 2001. 17:701-52; incorporated by reference in their entirety herein). Peroxisomes are small organelles lacking DNA and ribosomes and are lined by a single membrane. Peroxisomal proteins are encoded by nuclear genes, synthesized on ribosomes free in the cytosol, and then incorporated into pre-existing peroxisomes. During the lifespan of the cell, the peroxisomes may enlarge by the addition of protein and lipids, for example, and may eventually divide, forming new one peroxisomes.

The size and enzyme composition of peroxisomes may be varied. However, the peroxisomes may all contain enzymes that use molecular oxygen to oxidize various substrates, forming hydrogen peroxide (H2O2). Peroxisomes are known for H2O2-based respiration as well as fatty acid β-oxidation. Without being limiting, functions of the peroxisomes may include ether lipid (plasmalogen) synthesis and cholesterol synthesis, glyoxylate cycle in germinating seeds (“glyoxysomes”), photorespiration, glycolysis in trypanosomes (“glycosomes”), and methanol and/or amine oxidation and assimilation in yeast, for example.

Proteins that are directed for processing in the peroxisome may have C- and/or N-terminal targeting sequences direct entry of folded proteins into the peroxisomal matrix. After translation and release from cytosolic ribosomes, newly synthesized proteins targeted for the peroxisome, may fold into their mature conformation in the cytosol before import into the organelle. Folding may also occur by the assistance of chaperone proteins. Protein import into peroxisomes requires ATP hydrolysis, however, unlike some transport systems, there is no electrochemical gradient across the peroxisomal membrane. Tags for transport have been described previously (Purdue et al.). In some embodiments, the protein is folded by the assistance of chaperone proteins.

The uptake-targeting signal for some proteins targeted for the peroxisome is a Ser-Lys-Leu sequence (SKL in one-letter code) or a related sequence at the C-terminus of the protein. The SKL signal may bind to a soluble receptor protein, such as a peroxin, in the cytosol. There are several classes of peroxins (PTSs), such as PTS1 and PTS2. The resulting PTS1R-catalase complex then binds to a receptor protein. Cytosolic receptors have been identified, such as Pex5p for PTS1 and Pex7p for PTS2, in the peroxisome membrane, following which a targeted protein is transported inwards into the peroxisome. The SKL sequence is not cleaved from catalase after its entry into a peroxisome.

Without being limiting, matrix proteins may be synthesized as precursors with an N-terminal uptake-targeting sequence. Proteins with this type of uptake-targeting signal bind to a different cytosolic receptor protein named PTS2R that, like PTS1R, escorts the precursor protein to the Pex14p receptor on the peroxisomal membrane. Following import of such proteins, the N-terminal targeting sequence is cleaved. Peroxisomal membrane proteins are also synthesized on free polyribosomes and incorporated into peroxisomes after their synthesis. The signals that target proteins to the peroxisomal membrane do not contain an SKL sequence, but little else is known about this uptake process.

Other modifications in addition to prolyl hydroxylation are also achievable in the peroxisome. For example, protein substrates such as collagen can be glycosylated by co-importing a glycosyltransferase enzyme into the peroxisome through tagging with a peroxisome import tag. The peroxisome is naturally permeable to many small molecules that serve as modifying substrates by the modifying enzymes. Substrates that cannot freely diffuse into the peroxisome must be transported. Transport could be imported, either specifically or promiscuously, via a membrane protein targeted to the peroxisome membrane.

Modifications may also occur in the cytoplasmic surface of a peroxisome. Without being limiting, these modifications may include ubiquitination and phosphorylation, for example.

Chaperone proteins may also be tagged for peroxisome translocation. As such, chaperones may be used in the peroxisome for proper folding of the translocated protein in the peroxisome.

### Methods of Making Genetically Modified Cells for the Production of Modified Proteins

In some embodiments, a method of making a cell for producing a modified protein in a peroxisome is provided. The steps may comprise providing a cell, introducing a first nucleic acid into the cell, wherein the first nucleic acid comprises a first sequence encoding a heterologous protein fused to a peroxisome-targeting sequence and introducing a second nucleic acid into the cell, wherein the second nucleic acid comprises a second sequence encoding a heterologous modification enzyme fused to a peroxisome-targeting sequence. The cell may be a eukaryotic cell. In some embodiments, the introducing is performed in the presence of calcium chloride. In some embodiments, the introducing is performed by standard transformation techniques that are known to those of skill in the art, such as electroporation.

In some embodiments the cell is a yeast cell, such as Saccharomyces cerevisiae, Pichia pastoris and Ogataea polymorpha. For Pastoris cells, for example, the nucleic acid may have a promoter that allows induction of protein in the presence of methanol.

In some embodiments, the first and/or second nucleic acid comprises a promoter(s). In some embodiments, the promoter is constitutive or inducible.

In some embodiments, the peroxisome-targeting sequence comprises a sequence set forth in SEQ ID NO: 1 (SLK), SEQ ID NO: 2 (RLXXXXX(H/Q)L), or SEQ ID NO: 3 (LGRGRRSKL).

In some embodiments, the protein comprises a tag. In some embodiments, the tag is cleavable. The tag may be a tag that allows solubility of the protein or stability of a protein within the environment of the peroxisome.

In some embodiments, the method further comprises introducing a third nucleic acid into the cell, wherein the third nucleic acid comprises a third sequence encoding a second heterologous modification enzyme fused to a peroxisome-targeting sequence.

In some embodiments, the enzyme catalyzes a modification selected from a group of modifications selected from hydroxylation, oxidation, glycosyl transfer and isomerization.

In some embodiments, the enzyme comprises glycosyl transferases, isomerases (e.g., prolyl and disulfide), hydroxyl transferases (e.g., prolyl hydroxylases and lysyl oxidases).

In some embodiments, the enzyme is selected from a glycosyl transferase, an isomerase, a prolyl isomerase, hydroxyl transferase or a prolyl hydroxylase.

In some embodiments, the protein comprises collagen, gelatin or silk protein.

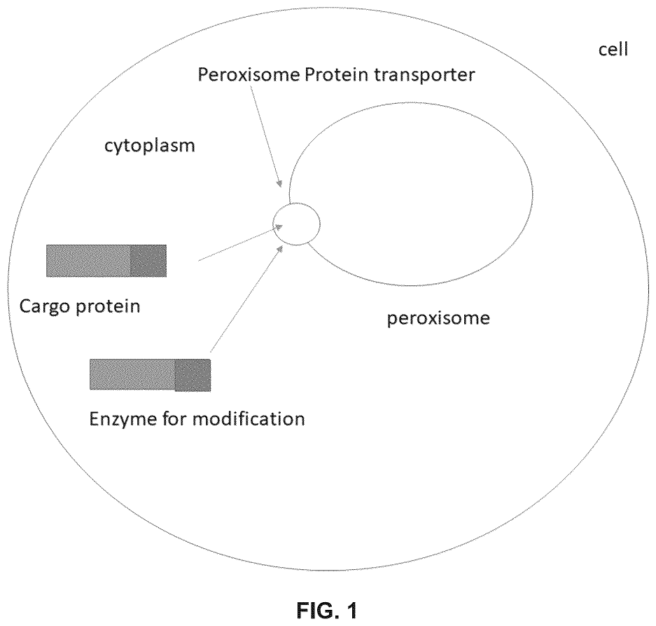

As shown in FIG. 1, the cell comprises nucleic acids encoding proteins and enzymes that are tagged for translocation in the peroxisome. Following translation, the C-terminal or N-terminal tags signal the translocation of the protein and enzyme into the peroxisome where they are further processed.

### Cells

In some embodiments, a eukaryotic cell for producing a protein in a peroxisome, manufactured by a method of any one of the embodiments described herein. In some embodiments, the cell comprises a first nucleic acid comprising a sequence encoding a heterologous protein fused to a peroxisome-targeting sequence and a second nucleic acid encoding a heterologous modification enzyme fused to a peroxisome-targeting sequence. In some embodiments, the cell comprises a peroxisome for producing a modified protein, wherein the eukaryotic cell is capable of expressing a heterologous protein fused to a peroxisome-targeting sequence, and a heterologous modification enzyme fused to a peroxisome-targeting sequence. In some embodiments, the cell comprises a peroxisome for producing a modified protein, wherein the eukaryotic cell comprises: a first nucleic acid sequence encoding a heterologous protein fused to a peroxisome-targeting sequence, and a second nucleic acid sequence encoding a heterologous modification enzyme fused to a peroxisome-targeting sequence (see FIG. 1)

In some embodiments, a eukaryotic cell is provided, comprising a peroxisome, for producing a modified protein, wherein the peroxisome comprises: a heterologous protein fused to a peroxisome-targeting sequence, and a heterologous modification enzyme fused to a peroxisome-targeting sequence.

In some embodiments, the protein is modified in the peroxisome. In some embodiments, the cell is Pastoris. In some embodiments, the peroxisome-targeting sequence comprises a sequence set forth in SEQ ID NO: 1, 2, or 3. The cell further comprises a third nucleic acid encoding a second protein fused to a peroxisome-targeting sequence.

The cells may be used for fermentation in standard fermentation broth. Those of skill in the art would appreciate the standard methods for growing cells for protein production. In some embodiments, fermentation may be performed in the presence of an inducing agent or in the presence of methanol.

In some embodiments, wherein a large amount of protein is required in large-scale production, the cells are grown in a fermenter. An advantage of Saccharomyces cerevisiae, Pichia pastoris and Ogataea polymorpha is that they may grow at a prolific growth rate. A fermenter may be used for preventing limitations due to pH control, oxygen limitation, nutrient limitation and temperature fluctuation. The fermenter enables dissolved oxygen (DO) levels to be raised, not just by increasing agitation, but by increasing air flow, by supplementing the air stream with pure oxygen. Nutrient limitation can also be minimized, since fermenters can be run in “fed mode” where fresh media or growth limiting nutrients can be pumped into the vessel at a rate that is capable of replenishing the nutrients that are depleted. The fermenter may also enable methanol flow rates to be controlled to condition the cells to the presence of the methanol, as well as provide methanol at the proper rate to allow addition of just enough methanol for protein synthesis while preventing excess methanol addition which may cause toxicity.

### Methods of Producing Modified Proteins

In some embodiments, a method of producing a modified protein in a eukaryotic cell containing a peroxisome is provided, wherein the eukaryotic cell expresses a heterologous modification enzyme fused to a peroxisome-targeting sequence. The method comprises providing a cell manufactured by the method of or a cell of any one of the embodiments herein, expressing a heterologous protein in the eukaryotic cell, wherein the heterologous protein is fused to a peroxisome-targeting sequence, and culturing the eukaryotic cell under conditions such that the heterologous modification enzyme modifies the heterologous protein in the peroxisome to produce a modified protein.

In some embodiments, a method of producing a modified protein in a eukaryotic cell containing a peroxisome is provided, wherein the eukaryotic cell expresses a heterologous modification enzyme fused to a peroxisome-targeting sequence. The method may comprise the steps of expressing a heterologous protein in a eukaryotic cell, wherein the heterologous protein is fused to a peroxisome-targeting sequence, and culturing the eukaryotic cell under conditions such that the heterologous modification enzyme modifies the heterologous protein in a peroxisome to produce a modified protein.

In some embodiments, a method of producing a modified protein in a eukaryotic cell containing method of producing a modified protein is provided. The method comprises the following steps: culturing a eukaryotic cell containing a peroxisome under conditions such that the modified protein is produced, wherein the eukaryotic cell expresses: a heterologous protein fused to a peroxisome-targeting sequence, and a heterologous modification enzyme fused to a peroxisome-targeting sequence, wherein the heterologous modification enzyme modifies the heterologous protein to produce the modified protein in the peroxisome under the culture conditions.

In some embodiments, a method of producing a modified protein in a eukaryotic cell containing method of increasing yield of a modified protein. In some embodiments, the eukaryotic cell is from Saccharomyces cerevisiae, Pichia pastoris or Ogataea polymorpha. The method comprise culturing a eukaryotic cell containing a peroxisome under conditions such that the modified protein is produced, wherein the eukaryotic cell expresses: a heterologous protein fused to a peroxisome-targeting sequence, wherein expression of the heterologous protein is under the influence of a promoter, and a heterologous modification enzyme fused to a peroxisome-targeting sequence; wherein the heterologous modification enzyme modifies the heterologous protein to produce the modified protein in the peroxisome under the culture conditions. In some embodiments, the method further comprises inducing production of the heterologous protein by addition of a chemical inducer. In some embodiments, the method further comprises increasing cargo of the peroxisome, wherein increasing cargo of the peroxisome is performed by providing oleic acid or methanol to the eukaryotic cell.

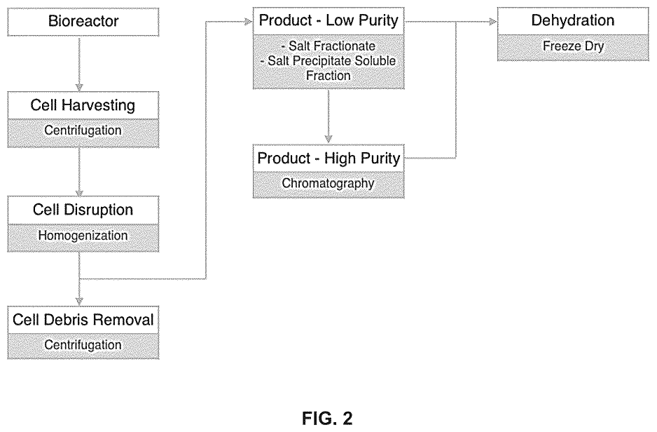

In some embodiments, cells are transformed with one or more nucleic acids as described herein (see, for example, FIG. 2). In some embodiments, the transformed cells are allowed to ferment. In some embodiments, after fermentation and inducing the protein for translation, which is followed by translocation, the cells are then harvested. Cells are centrifuged in some embodiments.

In some embodiments, the cells are then prepared for lysis. Homogenizers can be used to disrupt yeast cells. The homogenizers may lyse cells by pressurizing the cell suspension and suddenly releasing the pressure. This creates a liquid shear capable of lysing cells. Typical operating pressures for the older type of homogenizers, the French press and Manton-Gaulin homogenizer, are 6000-10,000 psi. Multiple (at least 3) passes are required to achieve a reasonable degree of lysis. The high operating pressures, however, may result in a rise in operating temperatures. Therefore, pressure cells are cooled (4° C.) prior to use in some embodiments. In addition to temperature control, care should be taken in some embodiments to avoid inactivating proteins by foaming. As such, pressure may be applied in increments. Lysis must also be done in the presence of inhibitors of proteases in some embodiments.

Modern homogenizers are more suited to lyse yeast cells since they can be operated at much higher pressures. An Avestin Emulsiflex-C5, for example, may be used to lyse Pichia pastoris cells at 30,000 psi (200 MPa).

Glass bead vortexing may also be used for cell lysis which disrupts yeast cells by agitation with glass beads (0.4-0.5 mm). Several cycles of agitation (30-60 sec) must be interspersed with cycles of cooling on ice to avoid overheating of the cell suspension. Breakage is variable, but can be well over 50% (up to 95%). Above the method is described for small volumes (up to 15 ml) but it can be scaled up to many liters using specialized apparatus.

Enzymatic lysis may also be used for lysing the cells. The enzymatic lysis of yeast cells is based on the digestion of the cell wall by a number of enzymes, such as zymolase and lyticase are the most widely used.

In some embodiments, following lysis, the supernatant is spun down and may also be filtered to remove particulate matter. Purification of peroxisomes is known to those of skill in the art and may be performed by gradient in a centrifuge. Peroxisomes may also be isolated by a commercial kit (e.g. Peroxisome Isolation Kit by Sigma Aldrich).

Following lysis of the peroxisomes, the lysate may be purified for the protein of interest. After bulk purification, the protein may be separated from the lysed peroxisomes. Techniques of purification are known to those of skill in the art. Depending on the type of protein and characteristics of the protein, different types of purification techniques may be considered. Without being limiting steps may be taken, such as ammonium sulfate precipitation, in order to isolate proteins by precipitation. Sucrose gradient centrifugation may also be used to separate different sizes of molecules in a sample. Size exclusion chromatography is largely used in non-denaturing or denaturing conditions depending if there are known methods to refold a protein. Proteins may also be separated based on their charge or hydrophobicity. If the protein is tagged, a protein may also be separated by affinity chromatography or immobilization to a column or resin.

Proteins of interest may then be analyzed by mass spectrometry for the modifications, for example. Proteins such as enzymes may also be analyzed in an activity assay.

Types of proteins may also be analyzed for translocation in the peroxisome. Methods to engineer proteins for stability are known to those of skill in the art. Without being limiting, this may include attaching cleavable tags in order to artificially change the pH of a protein, or creating several mutations in order to artificially change the pH of a protein that will be translocated into the peroxisome.

Other tags that may be considered are tags of proteins that are known to be translocated into the protein, or a domain thereof. As described in Purdue et al., the consensus sequence XX(K/R)(K/R)X₍₃₋₇₎(T/S)XX(D/E)X (SEQ ID NO: 4), where X is any amino acid, and where X₍₃₋₇₎ represents a range of 3-7 amino acids of any amino acid at the indicated position, is a conserved sequence in peroxisome proteins that may allow translocation or stability of a protein in the peroxisome.

In some embodiments of the methods, cells or compositions as described herein, a protein such as a heterologous protein fused to a peroxisome targeting sequence localizes to a peroxisome in a cell such as a eukaryotic or yeast cell. In some embodiments, an enzyme such as a modification enzyme fused to a peroxisome targeting sequence localizes, and/or co-localizes with the heterologous protein fused to a peroxisome targeting sequence, to a peroxisome in a cell such as a eukaryotic or yeast cell. In some embodiments, the protein and/or enzyme is fused to a peroxisome targeting signal such as PTS1 or ePTS1. For example, ePTS1 is the peroxisome targeting sequence in some embodiments. Examples of an ePTS1 tag and a nucleic acid sequence encoding an ePTS1 tag are provided in SEQ ID NO: 3 (LGRGRRSKL) and SEQ ID NO: 12 (TTGGGAAGAGGTAGAAGATCCAAATTG).

Various proteins and enzymes can be targeted to peroxisomes by use of a peroxisome targeting sequence. For example, proteins and enzymes with molecular weights between 1-5, 5-10, 10-25, 25-50, 50-75, 75-100 kDa 100-200 kDa, or 200-300 kDa, or higher, or a range of values encompassing any of the aforementioned kDa ranges can be targeted to a peroxisome with a peroxisome targeting sequence. In some embodiments, a nucleic acid with a sequence encoding the protein and/or enzyme to be targeted to the peroxisome, and encoding a peroxisome targeting sequence is transferred to a cell comprising a peroxisome, and the cell translates the protein and/or enzyme and transports it into the peroxisome. Additional examples of proteins and enzymes that may be targeted to peroxisomes include but are not limited to structural proteins, collagens, kinases, phosphatases, hydroxylases, isomerases, cleavage enzymes, fluorescent proteins, and hormones. In some embodiments, the protein and/or enzyme to be targeted includes a tag such as a fluorescent tag (for example, GFP, YFP, or CFP), a flag tag (for example DYKDDDDK where D=aspartic acid, Y=tyrosine, and K=lysine, SEQ ID NO: 5), or a histidine tag (for example, His-His-His-His-His-His, SEQ ID NO: 6). Such tags may be used for, without limitation, purifying and/or identifying a location of the protein and/or enzyme. Purification techniques may include but are not limited to affinity purification or use of ionic columns such as nickel columns to purify the protein and/or enzyme using the tag(s). Other tags that may be used include calmodulin (KRRWKKNFIAVSAANRFKKISSSGAL, SEQ ID NO: 7), HA (YPYDVPDYA, SEQ ID NO: 8), Myc (EQKLISEEDL, SEQ ID NO: 9), SBP (MDEKTTGWRGGHVVEGLAGELEQLRARLEHHPQGQREP, SEQ ID NO: 10), and/or Strp (WSHPQFEK, SEQ ID NO: 11) tags.

An example of a GFP tag is provided in SEQ ID NO: 13 (MRKGEELFTGVVPILVELDGDVNGHKFSVRGEGEGDATNGKLTLKFICTTGKLPVPWP TLVTTLTYGVQCFARYPDHMKQHDFFKSAMPEGYVQERTISFKDDGTYKTRAEVKFEG DTLVNRIELKGIDFKEDGNILGHKLEYNFNSHNVYITADKQKNGIKANFKIRHNVEDGSV QLADHYQQNTPIGDGPVLLPDNHYLSTQSVLSKDPNEKRDHMVLLEFVTAAGITHGMD ELYK). Some embodiments include a nucleic acid encoding a GFP tag, such as the nucleic acid sequence of SEQ ID NO: 14 (ATGCGTAAAGGCGAAGAGCTGTTCACTGGTGTCGTCCCTATTCTGGTGGAACTGGA TGGTGATGTCAACGGTCATAAGTTTTCCGTGCGTGGCGAGGGTGAAGGTGACGCAA CTAATGGTAAACTGACGCTGAAGTTCATCTGTACTACTGGTAAACTGCCGGTTCCTT GGCCGACTCTGGTAACGACGCTGACTTATGGTGTTCAGTGCTTTGCTCGTTATCCGG ACCATATGAAGCAGCATGACTTCTTCAAGTCCGCCATGCCGGAAGGCTATGTGCAGG AACGCACGATTTCCTTTAAGGATGACGGCACGTACAAAACGCGTGCGGAAGTGAAA TTTGAAGGCGATACCCTGGTAAACCGCATTGAGCTGAAAGGCATTGACTTTAAAGA GGACGGCAATATCCTGGGCCATAAGCTGGAATACAATTTTAACAGCCACAATGTTTA CATCACCGCCGATAAACAAAAAAATGGCATTAAAGCGAATTTTAAAATTCGCCACA ACGTGGAGGATGGCAGCGTGCAGCTGGCTGATCACTACCAGCAAAACACTCCAATC GGTGATGGTCCTGTTCTGCTGCCAGACAATCACTATCTGAGCACGCAAAGCGTTCTG TCTAAAGATCCGAACGAGAAACGCGATCATATGGTTCTGCTGGAGTTCGTAACCGCA GCGGGCATCACGCATGGTATGGATGAACTGTACAAA), or a fragment thereof.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Localization of Collagen Variants or P4HB to Peroxisome in Multiple Yeast Hosts

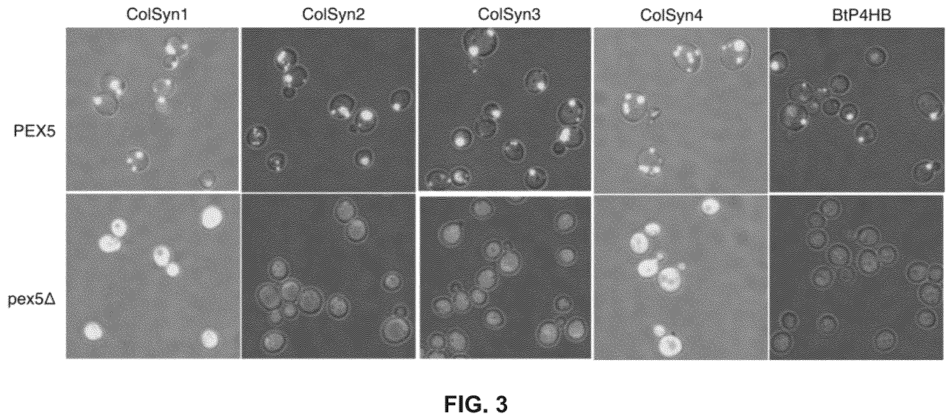

A GFP-x-ePTS1 construct was produced in which GFP was included for visualization of localization, ePTS1 was included for targeting to peroxisome), and where x is a protein of interest. Non-limiting examples of proteins of interest include synthetic collagen peptides COLsyn1a, COLsyn2, COLsyn3, COLsyn4, COLsyn5 and COLsyn6, and the protein disulfide-isomerase P4HB (see Table 1). In some embodiments, the P4HB is BantP4HB, ApmiP4HB, BtauP4HA1, BtauP4HB, BtP4HB, or GFP-B5P4HB-ePTS1, or a fragment or derivative thereof. Nucleic acids encoding these proteins of interest were included in separate constructs. The constructs produced peptides with each of the proteins of interest were imported into peroxisomes of wild-type (WT) S. cerevisiae strains visualized as fluorescent foci in the cell (FIG. 3). In strains that lack the peroxisome import receptor (pex5Δ), only diffuse cytoplasmic localization was seen. These results indicate that in some embodiments a peroxisome targeting peptide such as is described herein may be used to target a protein or enzyme to a peroxisome in a cell such as a yeast cell. Other non-limiting examples of proteins of interest and some examples of encoding nucleotide sequences are also shown in Table 1. In some embodiments, the protein of interest or an encoding nucleic acid consists of or comprises an amino acid or nucleotide sequence that is 50%, 55%, 60%, 65%, 70%, 75%, 80%, 85%, 90%, 91%, 92%, 93%, 94%, 95%, 96%, 97%, 98%, 99%, or 100%, or a range defined by any two of the aforementioned percentages, identical to any one or more of SEQ ID NOS: 15-70. Some embodiments include multiple proteins of interest that may be targeted to the peroxisome.

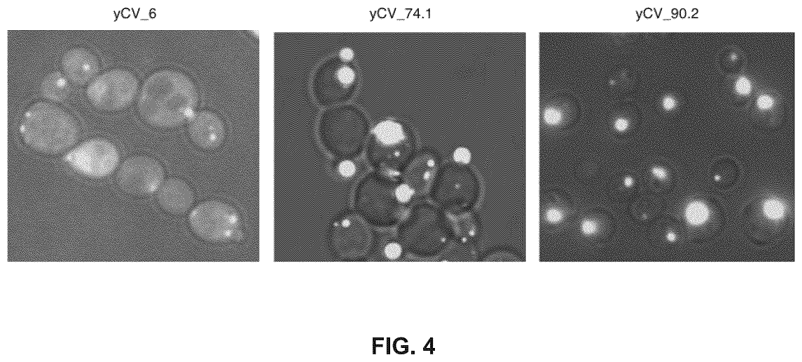

Various collagen variants have been observed to localize in multiple industrial yeast hosts (FIG. 4). Non-limiting examples of full-length collagen include AmCOL1A1, AmCOL1A2, BtCOL1A1, BtCOL1A2, and fragments thereof. Non-limiting examples of smaller collagen fragments include COLsyn1, COLsyn2, COLsyn3, COLsyn4, COLsyn, COLsyn5, and COLsyn6, BtCol1A1 403-11P, and BtCol1A1 403-OP. FIG. 4 shows the ePTS1-dependent fluorescence localization of GFP-collagen variants in three different industrial yeast hosts, PBH001, PBH002, PBH004. Common industrial yeast hosts include but are not limited to genera of Arxula, Candida, Hansenula, Kluyveromyces, Komagataella, Ogataea, Pichia, Saccharomyces, or Yarrowia.

The sizes of proteins observed to localize to the peroxisome range from 31 kDa (GFP-COLSyn1) to 195 kDa (BtCol1A2). Therefore, a substantial range of protein sizes can be imported into peroxisomes.

TABLE 1
Exemplary Nucleic Acid/Amino Acid Sequences

| SEQ | | |
|---|---|---|
| ID | | |
| NO: | Name | Sequence |
| 15 | Btau | ATGTTCAGCTTTGTGGACCTCCGGCTCCTGCTCCTCTTAGCGGCCACCGCC |
| | COL1A1 | CTCCTGACGCACGGCCAAGAGGAGGGCCAGGAAGAAGGCCAAGAAGAAG |
| | (DNA) | ACATCCCACCAGTCACCTGCGTACAGAACGGCCTCAGGTACCATGACCGA |
| | | GACGTGTGGAAACCCGTGCCCTGCCAGATCTGTGTCTGCGACAACGGCAA |
| | | CGTGCTGTGCGATGACGTGATCTGCGACGAACTTAAGGACTGTCCTAACGC |
| | | CAAAGTCCCCACGGACGAATGCTGCCCCGTCTGCCCCGAAGGCCAGGAAT |
| | | CACCCACGGACCAAGAAACCACCGGAGTCGAGGGACCGAAAGGAGACAC |
| | | TGGCCCCCGAGGCCCAAGGGGACCCGCCGGCCCCCCCGGCCGAGATGGCA |
| | | TCCCTGGACAACCTGGACTTCCCGGACCCCCTGGACCCCCCGGACCTCCCG |
| | | GACCCCCTGGCCTCGGAGGAAACTTTGCTCCCCAGTTGTCTTACGGCTATG |
| | | ATGAGAAATCAACAGGAATTTCCGTGCCTGGTCCCATGGGTCCTTCTGGTC |
| | | CTCGTGGTCTCCCTGGCCCCCCTGGCGCACCTGGTCCCCAAGGTTTCCAAG |
| | | GCCCCCCTGGTGAGCCTGGCGAGCCAGGAGCCTCAGGTCCCATGGGTCCC |
| | | CGTGGTCCCCCTGGCCCCCCTGGCAAGAACGGAGATGATGGCGAAGCTGG |
| | | AAAGCCTGGTCGTCCTGGTGAGCGCGGGCCTCCCGGACCTCAGGGTGCTC |
| | | GGGGATTGCCTGGAACAGCTGGCCTCCCTGGAATGAAGGGACACAGAGGT |
| | | TTCAGTGGTTTGGATGGTGCCAAGGGAGATGCTGGTCCTGCTGGCCCCAAG |
| | | GGCGAGCCTGGTAGCCCCGGTGAAAATGGAGCTCCTGGTCAGATGGGCCC |
| | | CCGTGGTCTGCCTGGTGAGAGAGGTCGCCCTGGAGCCCCTGGCCCTGCTGG |
| | | TGCTCGAGGAAATGATGGTGCGACTGGTGCTGCTGGGCCCCCTGGTCCCAC |
| | | TGGCCCCGCTGGTCCTCCTGGTTTCCCTGGTGCTGTGGGTGCTAAGGGTGA |
| | | AGGTGGTCCCCAAGGACCCCGAGGTTCTGAAGGTCCCCAGGGTGTACGTG |
| | | GTGAGCCTGGCCCCCCTGGCCCTGCTGGTGCTGCTGGCCCTGCTGGCAACC |
| | | CTGGTGCTGATGGACAGCCTGGTGCTAAAGGAGCCAATGGCGCTCCTGGT |
| | | ATTGCTGGTGCTCCTGGCTTCCCTGGTGCCCGAGGCCCCTCTGGACCCCAG |
| | | GGCCCCAGCGGCCCCCCTGGCCCCAAGGGTAACAGCGGTGAACCTGGTGC |
| | | TCCTGGCAGCAAAGGAGACACTGGCGCCAAGGGAGAACCCGGTCCCACTG |
| | | GTATTCAAGGCCCCCCTGGCCCCGCTGGGGAAGAAGGAAAGCGAGGAGCC |
| | | CGAGGTGAACCTGGACCTGCTGGCCTGCCTGGACCCCCTGGCGAGCGTGG |
| | | TGGACCTGGAAGCCGTGGTTTCCCTGGCGCCGACGGTGTTGCTGGTCCCAA |
| | | GGGTCCTGCTGGTGAACGCGGTGCTCCTGGCCCTGCTGGCCCCAAAGGTTC |
| | | TCCTGGTGAAGCTGGTCGCCCCGGTGAAGCTGGTCTGCCCGGTGCCAAGG |
| | | GTCTGACTGGAAGCCCTGGCAGCCCGGGTCCTGATGGCAAAACTGGCCCC |
| | | CCTGGTCCCGCCGGTCAAGATGGCCGCCCTGGACCTCCAGGCCCTCCCGGT |
| | | GCCCGTGGTCAGGCTGGCGTGATGGGTTTCCCTGGACCTAAAGGTGCTGCT |
| | | GGAGAGCCTGGAAAAGCTGGAGAGCGAGGTGTTCCTGGACCCCCTGGCGC |
| | | TGTTGGTCCTGCTGGCAAAGACGGAGAAGCTGGAGCTCAGGGACCCCCAG |
| | | GACCTGCTGGCCCCGCTGGTGAGAGAGGCGAACAAGGCCCTGCTGGCTCC |
| | | CCTGGATTCCAGGGTCTCCCCGGCCCTGCTGGTCCTCCTGGTGAAGCAGGC |
| | | AAACCTGGTGAACAGGGTGTTCCTGGAGATCTTGGTGCCCCCGGCCCCTCT |
| | | GGAGCAAGAGGCGAGAGAGGTTTCCCCGGCGAGCGTGGTGTGCAAGGGC |
| | | CGCCCGGTCCTGCAGGTCCCCGTGGGGCCAATGGTGCCCCTGGCAACGAT |
| | | GGTGCTAAGGGTGATGCTGGTGCCCCTGGAGCCCCCGGTAGCCAGGGTGC |
| | | CCCTGGCCTTCAAGGAATGCCTGGTGAACGAGGTGCAGCTGGTCTTCCAG |
| | | GCCCTAAGGGTGACAGAGGGGATGCTGGTCCCAAAGGTGCTGATGGTGCT |
| | | CCTGGCAAAGATGGCGTCCGTGGTCTGACTGGTCCCATCGGTCCTCCTGGC |
| | | CCCGCTGGTGCCCCTGGTGACAAGGGTGAAGCTGGTCCTAGTGGCCCAGC |
| | | CGGTCCCACTGGAGCTCGTGGTGCCCCCGGTGACCGTGGTGAGCCTGGTCC |
| | | CCCCGGCCCTGCTGGCTTCGCTGGCCCCCCTGGTGCTGATGGCCAACCTGG |
| | | TGCTAAAGGCGAACCTGGTGATGCTGGTGCTAAAGGTGACGCTGGTCCCC |
| | | CCGGCCCTGCTGGGCCCGCTGGACCCCCCGGCCCCATTGGTAACGTTGGTG |
| | | CTCCCGGACCCAAAGGTGCTCGTGGCAGCGCTGGTCCCCCTGGTGCTACTG |
| | | GTTTCCCAGGTGCTGCTGGCCGAGTCGGTCCCCCCGGCCCCTCTGGAAATG |
| | | CTGGACCCCCTGGCCCTCCTGGCCCTGCTGGCAAAGAAGGCAGCAAAGGC |
| | | CCCCGCGGTGAGACTGGCCCCGCTGGGCGTCCCGGTGAAGTCGGTCCCCCT |
| | | GGTCCCCCTGGCCCCGCTGGTGAGAAAGGAGCCCCTGGTGCTGACGGACC |
| | | TGCTGGAGCTCCTGGCACTCCTGGACCTCAAGGTATTGCTGGACAGCGTGG |
| | | TGTGGTCGGCCTGCCTGGTCAGAGAGGAGAAAGAGGCTTCCCTGGTCTTCC |
| | | TGGCCCCTCTGGTGAACCCGGCAAACAAGGTCCTTCTGGAGCAAGTGGTG |
| | | AACGTGGCCCCCCTGGTCCCATGGGCCCCCCTGGATTGGCTGGACCCCCTG |
| | | GCGAGTCTGGACGTGAGGGAGCTCCTGGTGCTGAAGGATCCCCTGGACGA |
| | | GATGGTTCTCCTGGCGCCAAGGGTGACCGTGGTGAGACCGGCCCTGCTGG |
| | | ACCTCCTGGTGCTCCTGGCGCTCCCGGTGCCCCCGGCCCTGTCGGACCTGC |
| | | CGGCAAGAGCGGTGATCGTGGTGAGACCGGTCCTGCTGGTCCTGCTGGTC |
| | | CCATTGGCCCCGTTGGTGCCCGTGGCCCCGCTGGACCCCAAGGCCCCCGTG |
| | | GTGACAAGGGTGAGACAGGCGAACAGGGCGACAGAGGCATTAAGGGTCA |
| | | CCGTGGCTTCTCTGGTCTCCAGGGTCCCCCCGGCCCTCCCGGCTCTCCTGGT |
| | | GAGCAAGGTCCTTCCGGAGCCTCTGGTCCTGCTGGTCCCCGCGGTCCCCCT |
| | | GGCTCTGCTGGTTCTCCCGGCAAAGATGGACTCAATGGTCTCCCAGGCCCC |
| | | ATCGGTCCCCCTGGGCCTCGAGGTCGCACTGGTGATGCTGGTCCTGCTGGT |
| | | CCTCCCGGCCCTCCTGGACCCCCTGGTCCCCCAGGTCCTCCCAGCGGCGGC |
| | | TACGACTTGAGCTTCCTGCCCCAGCCACCTCAAGAGAAGGCTCACGATGGT |
| | | GGCCGCTACTACCGGGCTGATGATGCCAATGTGGTCCGTGACCGTGACCTC |
| | | GAGGTGGACACCACCCTCAAGAGCCTGAGCCAGCAGATCGAGAACATCCG |
| | | GAGCCCTGAAGGCAGCCGCAAGAACCCCGCCCGCACCTGCCGTGACCTCA |
| | | AGATGTGCCACTCTGACTGGAAGAGCGGAGAATACTGGATTGACCCCAAC |
| | | CAAGGCTGCAACCTGGATGCCATTAAGGTCTTCTGCAACATGGAAACCGG |
| | | TGAGACCTGTGTATACCCCACTCAGCCCAGCGTGGCCCAGAAGAACTGGT |
| | | ATATCAGCAAGAACCCCAAGGAAAAGAGGCACGTCTGGTACGGCGAGAG |
| | | CATGACCGGCGGATTCCAGTTCGAGTATGGCGGCCAGGGGTCCGATCCTG |
| | | CCGATGTGGCCATCCAGCTGACTTTCCTGCGCCTGATGTCCACCGAGGCCT |
| | | CCCAGAACATCACCTACCACTGCAAGAACAGCGTGGCCTACATGGACCAG |
| | | CAGACTGGCAACCTCAAGAAGGCCCTGCTCCTCCAGGGCTCCAACGAGAT |
| | | CGAGATCCGGGCCGAGGGCAACAGCCGCTTCACCTACAGCGTCACCTACG |
| | | ATGGCTGCACGAGTCACACCGGAGCCTGGGGCAAGACAGTGATCGAATAC |
| | | AAAACCACCAAGACCTCCCGCTTGCCCATCATCGATGTGGCCCCCTTGGAC |
| | | GTTGGCGCCCCAGACCAGGAATTCGGCTTCGACGTTGGCCCTGCCTGCTTC |
| | | CTGTAA |
| 16 | Btau | MFSFVDLRLLLLLAATALLTHGQEEGQEEGQEEDIPPVTCVQNGLRYHDRDV |
| | COL1A1 | WKPVPCQ1CVCDNGNVLCDDVICDELKDCPNAKVPTDECCPVCPEGQESPTD |
| | (protein) | QETTGVEGPKGDTGPRGPRGPAGPPGRDGIPGQPGLPGPPGPPGPPGPPGLGG |
| | | NFAPQLSYGYDEKSTGTSVPGPMGPSGPRGLPGPPGAPGPQGFQGPPGEPGEPG |
| | | ASGPMGPRGPPGPPGKNGDDGEAGKPGRPGERGPPGPQGARGLPGTAGLPGM |
| | | KGHRGFSGLDGAKGDAGPAGPKGEPGSPGENGAPGQMGPRGLPGERGRPGA |
| | | PGPAGARGNDGATGAAGPPGPTGPAGPPGFPGAVGAKGEGGPQGPRGSEGPQ |
| | | GVRGEPGPPGPAGAAGPAGNPGADGQPGAKGANGAPGIAGAPGFPGARGPSG |
| | | PQGPSGPPGPKGNSGEPGAPGSKGDTGAKGEPGPTGIQGPPGPAGEEGKRGAR |
| | | GEPGPAGLPGPPGERGGPGSRGFPGADGVAGPKGPAGERGAPGPAGPKGSPG |
| | | EAGRPGEAGLPGAKGLTGSPGSPGPDGKTGPPGPAGQDGRPGPPGPPGARGQ |
| | | AGVMGFPGPKGAAGEPGKAGERGVPGPPGAVGPAGKDGEAGAQGPPGPAGP |
| | | AGERGEQGPAGSPGFQGLPGPAGPPGEAGKPGEQGVPGDLGAPGPSGARGER |
| | | GFPGERGVQGPPGPAGPRGANGAPGNDGAKGDAGAPGAPGSQGAPGLQGMP |
| | | GERGAAGLPGPKGDRGDAGPKGADGAPGKDGVRGLTGPIGPPGPAGAPGDK |
| | | GEAGPSGPAGPTGARGAPGDRGEPGPPGPAGFAGPPGADGQPGAKGEPGDAG |
| | | AKGDAGPPGPAGPAGPPGPIGNVGAPGPKGARGSAGPPGATGFPGAAGRVGP |
| | | PGPSGNAGPPGPPGPAGKEGSKGPRGETGPAGRPGEVGPPGPPGPAGEKGAPG |
| | | ADGPAGAPGTPGPQGIAGQRGVVGLPGQRGERGFPGLPGPSGEPGKQGPSGA |
| | | SGERGPPGPMGPPGLAGPPGESGREGAPGAEGSPGRDGSPGAKGDRGETGPA |
| | | GPPGAPGAPGAPGPVGPAGKSGDRGETGPAGPAGPIGPVGARGPAGPQGPRG |
| | | DKGETGEQGDRGIKGHRGFSGLQGPPGPPGSPGEQGPSGASGPAGPRGPPGSA |
| | | GSPGKDGLiNGLPGPIGPPGPRGRTGDAGPAGPPGPPGPPGPPGPPSGGYDLSFL |
| | | PQPPQEKAHDGGRYYRADDANWRDRDLEVDTTLKSLSQQIENIRSPEGSRK |
| | | NPARTCRDLKMCHSDWKSGEYWIDPNQGCNLDAIKVFCNMETGETCVYPTQ |
| | | PSVAQKNWYISKNPKEKRHVWYGESMTGGFQFEYGGQGSDPADVAIQLTFL |
| | | RLMSTEASQNITYHCKNSVAYMDQQTGNLKKALLLQGSNEIEIRAEGNSRFTY |
| | | SVTYDGCTSHTGAWGKTVIEYKTTKTSRLPIIDVAPLDVGAPDQEFGFDVGPA |
| | | CFL |
| 17 | Btau | ATGCTCAGCTTTGTGGATACGCGGACTTTGTTGCTGCTTGCAGTAACTTCG |
| | COL1A2 | TGCCTAGCAACATGCCAATCCTTACAAGAGGCAACTGCAAGAAAGGGCCC |
| | (DNA) | AAGTGGAGATAGAGGACCACGCGGAGAAAGGGGTCCACCAGGCCCACCA |
| | | GGCAGAGATGGTGATGACGGCATCCCAGGCCCTCCTGGCCCCCCTGGCCC |
| | | TCCTGGCCCCCCTGGTCTTGGCGGGAACTTTGCTGCTCAGTTTGATGCAAA |
| | | AGGAGGTGGCCCTGGACCAATGGGGCTGATGGGACCTCGCGGCCCTCCTG |
| | | GGGCTTCTGGAGCCCCTGGCCCTCAAGGTTTCCAGGGACCTCCGGGTGAGC |
| | | CTGGTGAACCTGGTCAGACTGGTCCTGCAGGTGCTCGTGGCCCGCCTGGCC |
| | | CTCCTGGCAAGGCTGGTGAGGATGGTCACCCTGGAAAACCTGGACGACCT |
| | | GGTGAGAGAGGGGTTGTTGGACCACAGGGTGCTCGTGGCTTTCCTGGAAC |
| | | TCCTGGACTCCCTGGCTTCAAGGGCATTAGGGGTCACAATGGTCTGGATGG |
| | | ATTGAAGGGACAGCCTGGTGCTCCAGGTGPGAAGGGTGAACCTGGTGCCC |
| | | CTGGTGAAAATGGAACTCCAGGTCAAACGGGAGCCCGTGGTCTTCCTGGT |
| | | GAGAGAGGACGTGTTGGTGCCCCTGGCCCAGCTGGTGCCCGTGGAAGTGA |
| | | TGGAAGTGTGGGTCCTGTGGGCCCTGCTGGTCCCATTGGGTCTGCTGGCCC |
| | | TCCAGGCTTCCCAGGTGCTCCTGGCCCCAAGGGTGAACTCGGACCTGTTGG |
| | | TAACCCTGGCCCTGCTGGTCCCGCGGGTCCCCGTGGTGAAGTGGGTCTCCC |
| | | AGGCCTTTCTGGCCCTGTCGGACCTCCTGGAAACCCCGGAGCCAATGGGCT |
| | | TCCTGGCGCTAAGGGTGCTGCTGGCCTTCCCGGTGTTGCTGGGGCTCCCGG |
| | | CCTCCCTGGACCCCGGGGTATTCCTGGCCCTGTTGGCGCTGCTGGTGCTAC |
| | | TGGCGCCAGAGGACTTGTTGGTGAGCCCGGCCCAGCTGGTTCGAAAGGAG |
| | | AGAGCGGCAACAAGGGCGAGCCTGGTGCTGTrGGGCAGCCAGGTCCTCCT |
| | | GGCCCCAGTGGTGAAGAAGGAAAGAGAGGCTCCACTGGAGAAATCGGAC |
| | | CCGCTGGCCCCCCAGGACCTCCTGGGCTGAGGGGAAATCCTGGCTCCCGT |
| | | GGTCTACCTGGAGCTGACGGCAGAGCTGGTGTCATGGGTCCTGCTGGTAG |
| | | CCGTGGTGCAACTGGCCCTGCTGGTGTGCGAGGTCCCAATGGAGATTCTGG |
| | | TCGCCCTGGAGAGCCTGGCCTCATGGGACCCCGAGGTTTCCCAGGTTCCCC |
| | | TGGAAATATCGGCCCAGCTGGTAAAGAAGGTCCTGTGGGTCTCCCTGGTAT |
| | | TGACGGCAGACCTGGGCCCATTGGCCCAGCGGGAGCAAGAGGAGAGCCTG |
| | | GCAACATTGGATTCCCTGGACCCAAAGGCCCCAGTGGTGATCCTGGCAAA |
| | | GCTGGTGAAAAAGGTCATGCTGGTCTTGCTGGTGCTCGGGGCGCTCCAGGT |
| | | CCCGATGGCAACAACGGTGCTCAGGGACCCCCTGGACTACAGGGTGTCCA |
| | | AGGTGGAAAAGGTGAACAGGGTCCTGCTGGTCCTCCAGGCTTCCAGGGTC |
| | | TGCCTGGCCCTGCAGGCACAGCTGGTGAAGCTGGCAAACCAGGAGAAAGG |
| | | GGTATCCCTGGTGAATTTGGTCTCCCTGGCCCTGCTGGTGCAAGAGGGGAG |
| | | CGGGGGCCCCCAGGTGAAAGTGGTGCTGCTGGGCCTACTGGGCCTATTGG |
| | | AAGCCGAGGTCCTTCTGGACCCCCAGGGCCTGATGGAAACAAGGGTGAAC |
| | | CGGGTGTGGTTGGCGCTCCAGGCACTGCTGGCCCATCTGGTCCTAGCGGAC |
| | | TCCCAGGAGAGAGGGGTGCGGCTGGCATTCCTGGAGGCAAGGGAGAAAA |
| | | GGGTGAAACTGGTCTCAGAGGTGACATTGGTAGCCCTGGTAGAGATGGTG |
| | | CTCGTGGTGCTCCTGGTGCTATTGGTGCTCCTGGCCCTGCTGGAGCCAATG |
| | | GGGACCGGGGTGAAGCTGGTCCCGCTGGCCCTGCTGGCCCTGCTGGTCCTC |
| | | GTGGTAGCCCTGGTGAACGTGGTGAGGTCGGTCCCGCTGGCCCCAACGGA |
| | | TTTGCTGGTCCTGCTGGTGCTGCTGGTCAACCTGGTGCTAAAGGAGAGAGA |
| | | GGAACCAAAGGACCCAAGGGTGAAAATGGTCCTGTTGGTCCCACAGGCCC |
| | | CGTTGGAGCTGCCGGTCCGTCTGGTCCAAATGGCCCACCTGGTCCTGCTGG |
| | | AAGTCGTGGTGATGGAGGGCCCCCTGGGGCTACTGGTTTCCCTGGTGCTGC |
| | | TGGACGGACTGGTCCCCCTGGACCCTCTGGTATCTCTGGCCCCCCTGGCCC |
| | | CCCTGGTCCTGCTGGTAAAGAAGGGGTTCGTGGGCCTCGTGGTGACCAAG |
| | | GTCCAGTTGGTCGAAGTGGAGAGACAGGTGCCTCTGGCCCTCCTGGCTTTG |
| | | TTGGTGAGAAGGGTCCCTCTGGAGAGCCTGGTACTGCTGGGCCTCCTGGA |
| | | ACCCCAGGTCCACAAGGCCTTCTTGGTGCTCCTGGTTTTCTGGGTCTCCCA |
| | | GGCTCTAGAGGTGAGCGrGGTCTACCAGGTGrCGCTGGATCTGTGGGTGA |
| | | ACCTGGCCCCCTCGGCATCGCAGGCCCACCTGGGGCCCGTGGTCCCCCTGG |
| | | TAATGTCGGTAATCCTGGCGTCAATGGTGCTCCTGGTGAAGCCGGTCGTGA |
| | | CGGCAACCCTGGGAATGACGGTCCCCCAGGCCGCGATGGTCAACCCGGAC |
| | | ACAAGGGGGAGCGTGGTTACCCCGGTAACGCAGGTCCTGTTGGTGCTGCC |
| | | GGTGCTCCTGGCCCTCAAGGCCCTGTGGGTCCCGTTGGTAAACACGGAAA |
| | | CCGTGGTGAACCGGGTCCTGCCGGTGCTGTTGGTCCTGCTGGTGCCGTTGG |
| | | CCCAAGAGGTCCCAGTGGCCCACAAGGTATTCGAGGTGACAAGGGAGAGC |
| | | CTGGTGATAAGGGTCCCAGAGGTCTTCCTGGCTTAAAGGGACACAATGGG |
| | | TTGCAAGGTCTCCCGGGTCTTGCTGGTCATCATGGCGATCAAGGTGCTCCC |
| | | GGTGCTGTGGGTCCCGCTGGTCCCAGGGGCCCTGCTGGTCCTTCTGGCCCC |
| | | GCTGGCAAAGACGGTCGCATTGGACAGCCTGGTGCAGTCGGACCTGCTGG |
| | | CATTCGTGGCTCTCAGGGTAGCCAAGGTCCTGCTGGCCCTCCTGGTCCCCC |
| | | TGGCCCTCCTGGACCTCCTGGCCCAAGTGGTGGTGGTTACGAGTTTGGTTT |
| | | TGATGGAGACTTCTACAGGGCTGACCAGCCTCGCTCACCAACTTCTCTCAG |
| | | ACCCAAGGATTATGAAGTTGATGCTACTCTGAAATCTCTCAACAACCAGAT |
| | | TGAGACCCTTCTTACTCCAGAAGGCTCTAGGAAGAACCCAGCTCGCACAT |
| | | GCCGAGACTTGAGACTCAGCCACCCAGAATGGAGCAGTGGTTACTACTGG |
| | | ATTGACCCTAACCAAGGATGTACTATGGATGCTATCAAAGTATACTGTGAT |
| | | TTCTCTACTGGCGAAACCTGCATCCGGGCTCAACCTGAAGACATCCCAGTC |
| | | AAGAACTGGTACAGAAATTCCAAGGCCAAGAAGCATGTCTGGGTAGGAGA |
| | | AAC1ATCAACGGTGGTACCCAGTTTGAA1ATAATGTTGAAGGAGTAACCA |
| | | CCAAGGAAATGGCTACCCAACTTGCCTTCATGCGTCTGCTGGCCAACCATG |
| | | CCTCTCAGAACATCACCTACCATTGCAAGAACAGCATTGCATACATGGATG |
| | | AGGAAACTGGCAACCTGAAAAAGGCTGTCATTCTGCAAGGATCCAATGAT |
| | | GTCGAACTTGTTGCCGAGGGCAACAGCAGATTCACTTACACTGTTCTTGTA |
| | | GATGGCTGCTCTAAAAAGACAAATGAATGGCAGAAGACAATCAT'TGAATA |
| | | TAAAACAAACAAGCCATCTCGCCTGCCTATCCTTGATATTGCACCTTTGGA |
| | | CATCGGTGGCGCTGACCAAGAAATCAGATTGAACATTGGCCCAGTCTGTTT |
| | | CAAATAA |
| 18 | Btau | MLSFVDTRTLLLLAVTSCLATCQSLQEATARKGPSGDRGPRGERGPPGPPGRD |
| | COL1A2 | GDDGIPGPPGPPGPPGPPGLGGNFAAQFDAKGGGPGPMGLMGPRGPPGASGA |
| | (protein) | PGPQGFQGPPGEPGEPGQTGPAGARGPPGPPGKAGEDGHPGKPGRPGERGVV |
| | | GPQGARGFPGTPGLPGFKGIRGHNGLDGLKGQPGAPGVKGEPGAPGENGTPG |
| | | QTGARGLPGERGRVGAPGPAGARGSDGSVGPVGPAGPIGSAGPPGFPGAPGP |
| | | KGELGPVGNPGPAGPAGPRGEVGLPGLSGPVGPPGNPGANGLPGAKGAAGLP |
| | | GVAGAPGLPGPRGIPGPVGAAGATGARGLVGEPGPAGSKGESGNKGEPGAVG |
| | | QPGPPGPSGEEGKRGSTGEIGPAGPPGPPGLRGNPGSRGLPGADGRAGVMGPA |
| | | GSRGATGPAGVRGPNGDSGRPGEPGLMGPRGFPGSPGNIGPAGKEGPVGLPGI |
| | | DGRPGPIGPAGARGEPGNIGFPGPKGPSGDPGKAGEKGHAGLAGARGAPGPD |
| | | GNNGAQGPPGLQGVQGGKGEQGPAGPPGFQGLPGPAGTAGEAGKPGERGIPG |
| | | EFGLPGPAGARGERGPPGESGAAGPTGPIGSRGPSGPPGPDGNKGEPGVVGAP |
| | | GTAGPSGPSGLPGERGAAGIPGGKGEKGETGLRGDIGSPGRDGARGAPGAIGA |
| | | PGPAGANGDRGEAGPAGPAGPAGPRGSPGERGEVGPAGPNGFAGPAGAAGQ |
| | | PGAKGERGTKGPKGENGPVGPTGPVGAAGPSGPNGPPGPAGSRGDGGPPGAT |
| | | GFPGAAGRTGPPGPSGISGPPGPPGPAGKEGLRGPRGDQGPVGRSGETGASGP |
| | | PGFVGEKGPSGEPGTAGPPGTPGPQGLLGAPGFLGLPGSRGERGLPGVAGSVG |
| | | EPGPLGIAGPPGARGPPGNVGNPGVNGAPGEAGRDGNPGNDGPPGRDGQPGH |
| | | KGERGYPGNAGPVGAAGAPGPQGPVGPVGKHGNRGEPGPAGAVGPAGAVG |
| | | PRGPSGPQGIRGDKGEPGDKGPRGLPGLKGHNGLQGLPGLAGHHGDQGAPG |
| | | AVGPAGPRGPAGPSGPAGKDGRIGQPGAVGPAGIRGSQGSQGPAGPPGPPGPP |
| | | GPPGPSGGGYEFGFDGDFYRADQPRSPTSLRPKDYEVDATLKSLNNQIETLLTP |
| | | EGSRKNPARTCRDLRLSHPEWSSGYYWIDPNQGCTMDAIKVYCDFSTGETCIR |
| | | AQPEDIPVKNWYRNSKAKKHVWVGETINGGTQFEYNVEGVTTKEMATQLAF |
| | | MRLLANHASQNITYHCKNSIAYMDEETGNLKKAVILQGSNDVELVAEGNSRF |
| | | TYTVLVDGCSKKTNEWQKTIIEYKTNKPSRLPILDIAPLDTGGADQEIRLNIGPV |
| | | CFK |
| 19 | Amis | ATGTTCAGCTTTGTGGATTCTCGGTTACTGCTGTTGATAGCAGCGACTGTA |
| | COL1A1 | CTACTCACCAAAGGTCAAGGAGAAGAAGACATTCAAACTGGAAGCTGCAT |
| | (DNA) | ACAGGATGGACTAGCGTACAACAACACAGACGTATGGAAACCCGAGCCCT |
| | | GCCAGATCTGCGTATGCGACAATGGCAACATCCTGTGTGACGATGTCATCT |
| | | GTGATGATACCTCGGACTGTACCAATGCTGAGATCCCCTTTGGAGAATGCT |
| | | GTCCCATCTGTCCTGACACCGCTGGCTCTTCTACCTACCCCAAATCCACTG |
| | | GAGTAGAGGGTCCTAAGGGAGACACTGGCCCCAGAGGACAGAGGGGACT |
| | | CCCAGGCCCACCTGGCAGAGATGGCATTCCTGGACAGCCTGGTCTCCCTGG |
| | | ACTCCCAGGACCTCCAGGCCCTCCTGGCCTTGGTGGAAACTTCGCTCCTCA |
| | | AATGGCTTACGGTTACGGAGATGAAACCAAATCTGCTGGCATTTCTGTCCC |
| | | TGGACCCATGGGTCCAGCTGGCCCCCGTGGTCTCCCCGGCCCCCCTGGTTC |
| | | TCCTGGTCCTCAAGGTTTCCAAGGTCCTCCTGGAGAGCCTGGAGAGCCTGG |
| | | TGCTTCAGGTCCAATGGGTCCCCGTGGTCCAGCCGGCCCCCCTGGCAAGAA |
| | | CGGAGATGATGGTGAAGCTGGAAAGCCCGGCCGTCCCGGTGAGCGCGGCC |
| | | CTCCTGGCCCCCAGGGTGCACGTGGTCTGCCCGGAACTGCTGGCCTGCCAG |
| | | GCATGAAGGGTCACAGAGGTTTCAGTGGTCTGGATGGTGCTAAGGGTGAT |
| | | GCTGGTCCATCCGGCCCCAAGGGTGAGCCTGGTAGCCCTGGTGAGAACGG |
| | | AGCTCCTGGACAAATGGGCCCTCGTGGTCTTCCCGGTGAGAGAGGCCGCC |
| | | CTGGTCCATCTGGCCCTGCTGGTGCTCGTGGTAACGATGGTAGTCCTGGTG |
| | | CTGCTGGCCCTCCAGGTCCAACTGGCCCAGCTGGCCCCCCTGGCTTCCCTG |
| | | GTGCTGCTGGTGCTAAGGGTGAAACTGGTCCTCAAGGTTCTCGTGGTAGTG |
| | | AAGGCCCACAGGGTGCTCGTGGTGAGCCTGGTCCTCCTGGCCCTGCTGGTG |
| | | CTGCTGGTCCTGCTGGCAACCCTGGTTCTGATGGTCAAGCTGGTGCCAAAG |
| | | GTGCAACTGGTGCTCCTGGTATTGCTGGTGCTCCTGGCTTCCCTGGCGCTC |
| | | GTGGCCCATCTGGACCCCAGGGTCCCAGCGGTGCTCCTGGCCCCAAGGGT |
| | | AACAGTGGTGAACCCGGTGCTCAAGGCAACAAGGGAGACACTGGTGCAA |
| | | AAGGAGAGCCTGGTCCTGCTGGTGTCCAAGGCCCACCTGGTCCAGCTGGT |
| | | GAAGAAGGCAAGAGAGGAGCCCGTGGTGAGCCCGGCCCTGGAGGTCTTCC |
| | | TGGCCCTGCTGGCGAACGTGGTGCTCCTGGAAGCCGTGGTTTCCCTGGCGC |
| | | TGATGGCATTTCTGGTCCCAAGGGTCCCCCTGGTGAACGTGGTTCCCCTGG |
| | | CCCTGCTGGTCCCAAAGGATCTACTGGTGAATCTGGACGCCCTGGTGAGCC |
| | | TGGTCTCCCTGGTGCCAAGGGTCTTACTGGAAGCCCAGGTAGCCCAGGTCC |
| | | TGATGGCAAGACTGGTCCACCTGGCCCCGCTGGTCAAGATGGTCGCCCAG |
| | | GACCCCCAGGCCCACCTGGTGCCAGAGGTCAGGCTGGTGTGATGGGTTTC |
| | | CCTGGACCTAAAGGTGCTGCTGGTGAGCCTGGCAAACCTGGTGAGAGAGG |
| | | AGCTCCTGGACCCCCTGGTGCTGTTGGCGCAGCTGGTAAGGATGGTGAAG |
| | | CTGGTGCCCAAGGTTCTCCTGGCGCTGCTGGTCCTGCTGGAGAGAGAGGTG |
| | | AACAAGGTCCTGCTGGTGCTCCTGGATTCCAGGGTCTGCCCGGTCCTGCTG |
| | | GCCCATCTGGTGAATCTGGCAAGCCTGGTGAACAGGGTGTTCCTGGAGAT |
| | | GCTGGTGCTCCTGGTCCAGCTGGTGCAAGAGGCGAGAGAGGTTTCCCTGG |
| | | TGAGCGTGGTGTCCAAGGTCAACCAGGTCCACAGGGTCCACGTGGTGCTA |
| | | ACGGTGCTCCCGGTAACGATGGTGCTAAGGGTGATGCTGGTGCTCCTGGTG |
| | | CTCCTGGTGGCCAAGGTCCTCCCGGTCTGCAGGGTATGCCTGGTGAGCGTG |
| | | GTGCTGCTGGTCTGCCTGGTTCCAAGGGTGACAGAGGCGATCCTGGTCCCA |
| | | AAGGCACTGATGGTGCTCCTGGCAAAGATGGCGTCAGAGGTCTAACTGGC |
| | | CCTATTGGTCCTCCTGGCCCAGCTGGTGCCCCTGGTGACAAGGGTGAAGCT |
| | | GGTCCTTCTGGCCCTGCTGGTCCCACTGGTTCTCGTGGTGCCCCTGGAGAT |
| | | CGTGGTGAGCCTGGTCCACCTGGCCCTGCTGGATTCGCTGGTCCCCCTGGT |
| | | GCTGATGGACAACCTGGTGCTAAAGGTGAATCTGGTGATGCTGGTGCTAA |
| | | AGGTGATGCTGGTCCTCCAGGCCCTGCTGGACCCACTGGTGCTCCTGGACC |
| | | TTCTGGCGCTGTTGGTGCTCCTGGACCCAAAGGTGCTCGTGGTAGTGCTGG |
| | | ACCCCCTGGTGCTACTGGTTTCCCTGGTGCTGCTGGAAGAGTTGGTCCACC |
| | | TGGCCCTGCTGGTAACGTCGGTCTTCCTGGCCCATCAGGCCCCAGTGGAAA |
| | | AGAAGGCTCTAAAGGACCCCGTGGTGAGACTGGCCCTGCTGGACGCCCCG |
| | | GTGAACCTGGACCTGCTGGCCCACCAGGACCTTCTGGCGAGAAGGGCTCT |
| | | CCTGGTGGTGATGGTCCCGCTGGTGCTCCTGGTACTCCAGGCCCACAGGGT |
| | | ATTGCTGGACAGCGTGGTGTAGTTGGTCTTCCTGGACAGAGAGGCGAGAG |
| | | AGGTTTCCCTGGTCTCCCCGGCCCATCTGGCGAACCTGGCAAACAAGGTCC |
| | | ATCTGGCTCCTCTGGTGAACGCGGTCCTCCTGGTCCAATGGGACCACCTGG |
| | | CTTGGCTGGACCTCCTGGTGAAGCTGGACGTGAGGGTGCTCCTGGTTCTGA |
| | | AGGTGCTCCTGGTCGCGATGGCGCTGCTGGTCCCAAGGGTGACCGTGGTG |
| | | AGACTGGCCCCTCTGGTCCTCCTGGTGCTCCCGGTGCCCCTGGAGCTCCTG |
| | | GCCCTATTGGCCCTGCTGGCAAGAATGGAGATCGTGGTGAGACTGGTCCTT |
| | | CTGGTCCTGCTGGCCCTGCCGGTCCTGCTGGTGCTCGTGGTCCTGCTGGTC |
| | | CACAAGGTGCCCGTGGTGACAAAGGTGAAACTGGAGAACATGGTGACAG |
| | | AGGCATGAAGGGTCACAGAGGATTCCCTGGTCCCCAGGGTCCCTCTGGTC |
| | | CTGCTGGCTCTCCTGGTGAACAAGGTCCTTCTGGAGCTTCCGGCCCTGCTG |
| | | GTCCAAGAGGTCCTCCTGGCTCTGCTGGCACCCCTGGCAAAGATGGTCTGA |
| | | ATGGTCTCCCTGGCCCTATTGGTCCACCTGGTCCCCGGGGTCGCACTGGTG |
| | | ATGTTGGTCCTGCTGGTCCCCCTGGACCTCCTGGGCCCCCAGGTCCTCCTG |
| | | GTGCACCCAGCGGCGGCTTTGACTTCAGCTTCATGCCCCAGCCTCCTCAGG |
| | | AGAAAGCCCATGATCCTGGCCGCTACTACAGAGCTGATGACGCCAACGTG |
| | | ATGCGTGACCGTGACCTGGAGGTGGACACCACCCTCAAGAGCCTGAGCCA |
| | | GCAGATCGAGAACATCCGCAGCCCCGAGGGCACCAGGAAGAACCCTGCCC |
| | | GCACCTGCCGTGACCTGAAGATGTGCCACAATGACTGGAAGAGCGGCGAG |
| | | TACTGGATTGACCCCAACCAGGGCTGCAATCTGGATGCCATCAAGGTCTAC |
| | | TGTAACATGGAGACTGGCGAGACTTGCGTCCACCCAACCCAGGCCACCAT |
| | | CGCTCAGAAGAACTGGTACATGAGCAAGAACCCCAAGGAGAAGAAACAC |
| | | ATCTGGTTTGGCGAGACAATGAGCGATGGCTTCCAGTTCGAATATGGTGG |
| | | GGAGGGCTCCAACCCAGCTGACGTTGCCATCCAACTGACCTTCCTGCGCCT |
| | | GATGTCCACTGAGGCCTCCCAGAACATCACCTACCACTGCAAGAACAGCG |
| | | TGGCTTACATGGACCAGGAGACTGGCAACCTGAAGAAGGCTCTGCTCCTT |
| | | CAGGGCTCCAACGAGATCGAGATCAGAGCAGAAGGCAACAGCCGCTTCAC |
| | | CTATGGAGTCACTGAGGATGGCTGCACAACTCACACCGGTGCCTGGGGCA |
| | | AGACAGTCATTGAATACAAAACAACAAAAACCTCTCGCCTGCCCGTCATT |
| | | GACGTGGCTCCCATGGACGTTGGAGCACAAGATCAGGAATTCGGAATTGT |
| | | CATCGGACCTGTCTGCTTCTTGTAA |
| 20 | Amis | MFSFVDSRLLLLIAATVLLTKGQGEEDIQTGSCIQDGLAYNNTDVWKPEPCQI |
| | COL1A1 | CVCDNGNILCDDVICDDTSDCTNAEIPFGECCPICPDTAGSSTYPKSTGVEGPK |
| | (protein) | GDTGPRGQRGLPGPPGRDGIPGQPGLPGLPGPPGPPGLGGNFAPQMAYGYGD |
| | | ETKSAGISVPGPMGPAGPRGLPGPPGSPGPQGFQGPPGEPGEPGASGPMGPRGP |
| | | AGPPGKNGDDGEAGKPGRPGERGPPGPQGARGLPGTAGLPGMKGHRGFSGL |
| | | DGAKGDAGPSGPKGEPGSPGENGAPGQMGPRGLPGERGRPGPSGPAGARGN |
| | | DGSPGAAGPPGPTGPAGPPGFPGAAGAKGETGPQGSRGSEGPQGARGEPGPPG |
| | | PAGAAGPAGNPGSDGQAGAKGATGAPGIAGAPGFPGARGPSGPQGPSGAPGP |
| | | KGNSGEPGAQGNKGDTGAKGEPGPAGVQGPPGPAGEEGKRGARGEPGPGGL |
| | | PGPAGERGAPGSRGFPGADGISGPKGPPGERGSPGPAGPKGSTGESGRPGEPGL |
| | | PGAKGLTGSPGSPGPDGKTGPPGPAGQDGRPGPPGPPGARGQAGVMGFPGPK |
| | | GAAGEPGKPGERGAPGPPGAVGAAGKDGEAGAQGSPGAAGPAGERGEQGPA |
| | | GAPGFQGLPGPAGPSGESGKPGEQGVPGDAGAPGPAGARGERGFPGERGVQG |
| | | QPGPQGPRGANGAPGNDGAKGDAGAPGAPGGQGPPGLQGMPGERGAAGLP |
| | | GSKGDRGDPGPKGTDGAPGKDGVRGLTGPIGPPGPAGAPGDKGEAGPSGPAG |
| | | PTGSRGAPGDRGEPGPPGPAGFAGPPGADGQPGAKGESGDAGAKGDAGPPGP |
| | | AGPTGAPGPSGAVGAPGPKGARGSAGPPGATGFPGAAGRVGPPGPAGNVGLP |
| | | GPSGPSGKEGSKGPRGETGPAGRPGEPGPAGPPGPSGEKGSPGGDGPAGAPGT |
| | | PGPQGIAGQRGVVGLPGQRGERGFPGLPGPSGEPGKQGPSGSSGERGPPGPMG |
| | | PPGLAGPPGEAGREGAPGSEGAPGRDGAAGPKGDRGETGPSGPPGAPGAPGA |
| | | PGPIGPAGKNGDRGETGPSGPAGPAGPAGARGPAGPQGARGDKGETGEHGDR |
| | | GMKGHRGFPGPQGPSGPAGSPGEQGPSGASGPAGPRGPPGSAGTPGKDGLNG |
| | | LPGPIGPPGPRGRTGDVGPAGPPGPPGPPGPPGAPSGGFDFSFMPQPPQEKAHD |
| | | PGRYYRADDANVMRDRDLEVDTTLKSLSQQIENIRSPEGTRKNPARTCRDLK |
| | | MCHNDWKSGEYWIDPNQGCNLDAIKVYCNMETGETCVHPTQATIAQKNWY |
| | | MSKNPKEKKHIWFGETMSDGFQFEYGGEGSNPADVAIQLTFLRLMSTEASQNI |
| | | TYHCKNSVAYMDQETGNLKKALLLQGSNEIEIRAEGNSRFTYGVTEDGCTTH |
| | | TGAWGKTVIEYKTTKTSRLPVIDVAPMDVGAQDQEFGIVIGPVCFL |
| 21 | Amis | ATGCTCAGCTTTGTGGATACACGGATTTTGTTGCTGCTCGCAGTAACTTCG |
| | COL1A2 | TACCTAGCAACATGTCAACAAGCAAAFGAGGCAACFGCAGGACGGAAGG |
| | (DNA) | GCCCAAGAGGAGACAAAGGGCCACAGGGAGAAAGGGGTCCACCAGGTCC |
| | | ACCAGGCAGAGATGGTGAAGATGGTCCACCAGGGCCTCCAGGGCCCCCTG |
| | | GTCCTCCAGGTCTTGGCGGAAACTTTGCTGCTCAGTATGACGGAGCAAAA |
| | | GCAGGTGACTATGGCFCAGGACCAATGGGTTTAATGGGACCCAGAGGCCC |
| | | ACCTGGAACAAGTGGACCTCCTGGTCCTCCTGGCTTCCAAGGACCTCATGG |
| | | TGAGCCTGGTGAACCTGGTCAAACAGGTCCCCAGGGTCCCCGTGGTCCATC |
| | | TGGTCCTCCTGGAAAGGCTGGTGAAGATGGCCATCCTGGAAAATCTGGAC |
| | | GATCTGGTGAGAGGGGCGTCTCTGGTCCTCAGGGTGCTCGTGGTTTCCCTG |
| | | GAACTCCTGGTCTGCCTGGCTTTAAGGGAATTAGAGGACACAATGGTCTG |
| | | GATGGTCAGAAGGGACAACCTGGTACTCCAGGCATTAAGGGFGAATCCGG |
| | | TGCCCCTGGTGAAAATGGTACCCCAGGACAATCTGGTGCTCGTGGCCTTCC |
| | | CGGTGAAAGAGGAAGAATTGGTGCACCTGGCCCAGCTGGTGCCCGTGGCA |
| | | GCGATGGTAGCACTGGTCCCACTGGTCCTGCTGGCCCTATCGGTTCTGCTG |
| | | GTGCTCCAGGTTTCCCAGGTGCTCCTGGAGCCAAGGGTGAAATTGGAGCT |
| | | GCTGGTAATGFAGGTCCTTCTGGCCCTGCTGGFCCACGAGGAGAGGCTGG |
| | | ACTTCCTGGTTCTTCTGGTCCCGTTGGCCCTCCTGGAAACCCTGGTTCTAAT |
| | | GGTCTTGCTGGTGCTAAAGGTGCAACTGGTCTTCCTGGTGTTGCTGGTGCT |
| | | CCTGGCTTGCCTGGTCCACGTGGTATTCCTGGACCTTCTGGCCCTGCCGGA |
| | | GCTGCTGGCACCAGAGGTCTTGTTGGTGAACCAGGCCCTGCTGGTGCCAA |
| | | GGGAGAAAGTGGTAACAAGGGTGAACCCGGTGCTGCTGGTCCATCAGGTC |
| | | CCGCTGGTCCAAGTGGTGAAGAAGGCAAGAAAGGTACFACTGGTGAACCT |
| | | GGCTCTTCTGGCCCCCCTGGTCCAGCTGGTCTAAGAGGCGTTCCTGGATCT |
| | | CGTGGTCTCCCTGGAGCTGACGGCAGAGCTGGTGTTATGGGACCTGCTGGC |
| | | AGCCGTGGTGCTACTGGTCCTGCTGGTGCTAAAGGTCCTAGTGGTGATAAT |
| | | GGTCGCCCTGGTGAGCCTGGCCTTATGGGTCCAAGAGGTCTCCCTGGTCAA |
| | | CCTGGAAGCTCAGGCCCTGCTGGCAAGGAAGGTCCTGTTGGTTTCCCTGGT |
| | | GCAGATGGTAGAGTTGGCCCAACTGGFCCAGCTGGFGCAAGAGGTGAGCC |
| | | TGGCAACATTGGATTCCCTGGACCCAAAGGCCCCACTGGTGACCCTGGCA |
| | | AACCTGGTGACAGAGGCCATGCTGGTCTTGCTGGTGCTCGGGGTGCGCCTG |
| | | GTCCTGAGGGCAACAATGGGGCTCAAGGTCCTCCTGGTGTTGCTGGCAAC |
| | | CCTGGTGCAAAAGGTGAACAAGGFCCAGCTGGFCCTCCCGGTTTCCAGGG |
| | | TCTCCCAGGCCCCTCAGGTCCAGCTGGTGAAGCTGGCAAACCAGGTGAAA |
| | | GGGGTATGGCTGGTGAATTTGGTGCCCCTGGCCCTGCGGGTTCAAGAGGT |
| | | GAACGTGGTCCTCCAGGCGAAAGTGGTGCTGTTGGTCCTGTAGGTCCCATT |
| | | GGAAGCCGTGGTCCATCTGGTCCACCAGGCACTGATGGCAACAAGGGTGA |
| | | ACCTGGTAATGTTGGTAATGCTGGTACTGCAGGCCCCTCTGGCGCTGGTGG |
| | | AGCCCCAGGAGAGAGAGGCATTGCTGGTATTCCAGGACCCAAGGGTGAAA |
| | | AGGGTGCTACAGGTCTGAGAGGGGATACTGGCGCAACAGGAAGAGATGG |
| | | TGCTCGTGGTGCTCCTGGTGCTATTGGAGCCCCTGGCCCCGCTGGTGGAGC |
| | | TGGTGAGCGGGGTGAAGGTGGTCCTGCTGGTGCTGCTGGCCCTTCTGGTGC |
| | | CCGTGGTATTCCTGGTGAACGTGGTGAGCCTGGTCCTGCTGGCCCTACTGG |
| | | ATTTGCTGGACCTGCTGGTGCAGCTGGCCAACCTGGTGCTAAAGGTGAAC |
| | | GAGGTACAAAAGGACCCAAGGGTGAAAATGGTCCACAAGGTGCTGTTGGC |
| | | CCAGTTGGTTCTTCTGGACCATCAGGTCCTGTTGGTGCCTCTGGTCCTGCTG |
| | | GTCCTCGTGGTGATGGTGGTCCTCCTGGTGTCACTGGTTTCCCTGGAGCTG |
| | | CTGGCAGAACTGGTCCTCCCGGCCCCTCTGGTATCACTGGCCCCCCTGGTC |
| | | CCCCTGGCTCAGCTGGCAAAGATGGTATGAGAGGCCCACGTGGTGATACT |
| | | GGTCCAGTTGGCCGCACTGGAGAACAAGGCATTGTTGGCCCACCTGGCTTC |
| | | AGTGGTGAGAAAGGTCCATCTGGAGAGCCTGGTGCTGCTGGTCCCCCTGG |
| | | TACCCCAGGTCCTCAGGGTATTCTTGGTGCTCCTGGTATCCTTGGTCTGCCT |
| | | GGCTCTCGGGGAGAACGTGGTCTTCCAGGCATCTCTGGAGCAACAGGTGA |
| | | ACCAGGTCCTCTTGGTATTTCCGGTCCTCCTGGTGCACGTGGTCCCTCTGGC |
| | | CCCGTGGGTTCTGCTGGTCTGAATGGTGCCCCTGGTGAAGCTGGCCGTGAT |
| | | GGCAATCCTGGCCATGATGGTGCTCCAGGCCGTGATGGTGCTCCTGGTTTC |
| | | AAGGGTGAGCGTGGTGCTCCTGGGAACAATGGACCTGCTGGTGCTGTTGG |
| | | TGCTCCTGGCGCCCATGGTCAAGTTGGTCCTGCTGGAAAGCCTGGAAATCG |
| | | TGGTGATCCTGGTCCTGTTGGTCCTTCTGGTCCTGCTGGTGCTTTTGGTGCA |
| | | AGGGGTCCTTCTGGCCCACAAGGTGCACGTGGTGAGAAGGGAGAAACAGG |
| | | TGAAAAGGGACACAGAGGTATGCCTGGATTTAAGGGGCACAATGGACTTC |
| | | AGGGTCTGCCTGGTCTTGCTGGCCAACATGGAGATCAAGGTCCTCCAGGTT |
| | | CTACTGGCCCCGCTGGCCCAAGGGGTCCCTCTGGTCCTTCTGGTCCTGCTG |
| | | GAAAAGATGGTCGCAATGGACTCCCTGGCCCTATTGGACCTGCTGGTGTGC |
| | | GTGGTTCTCAGGGTAGCCAAGGTCCTTCGGGTCCACCTGGCCCACCTGGTC |
| | | TCCCTGGTCCCCCTGGTGCAAATGGTGGTGGATACGAAGTTGGCTATGATC |
| | | TTGAATACTACCGGGCTGATCAGCCTGCTCTCAGACCTAAGGACTATGAAG |
| | | TTGATGCCACTCTGAAAACATTGAACAACCAAATTGAGACCCTCCTGACCC |
| | | CAGAAGGCTCCAGGAAGAACCCAGCTCGCACCTGCCGTGACCTGAGACTC |
| | | AGCCACCCAGAATGGACCAGTGGTTTCTACTGGATTGATCCCAACCAGGG |
| | | CTGTACTATGGATGCCATTAGAGTGTATTGTGACTTCTCCACTGGTGAGAC |
| | | TTGCATACATGCCAATCTAGAAAACATCCCCACTAAGAACTGGTATGTCAG |
| | | CAAGAACTCCAAGGAAAAGAAGCACATGTGGTTTGGTGAAACTATCAATG |
| | | GTGGTACCCAGTTTGAATATAACGATGAAGGAGTGACTTCCAAGGACATG |
| | | GCTACCCAACTTGCCTTCATGCGTCTGCTGGCCAACCATGCCTCCCAGAAC |
| | | ATCACCTACCACTGCAAGAACAGTATTGCATACATGGATGAAGAAACTGG |
| | | CAACCTTAAGAAGGCTGTAATACTGCAGGGATCCAATGATGTTGAACTAC |
| | | GAGCTGAAGGCAACAGCAGATTCACTTTCAGTGTTCTGGAAGATGGCTGC |
| | | TCTAGAAAGAACAACGCATGGGGCAAAACAATCATTGAATATAGAACAAA |
| | | CAAACCATCTCGCTTGCCCATCCTTGACATTGCACCTTTGGACATTGGTGG |
| | | AGCTGATCAAGAATTCGGTTTGGACATTGGCCCAGTCTGTTTCAAATGA |
| 22 | Amis | MLSFVDTRILLILAVTSYLATCQQANEATAGRKGPRGDKGPQGERGPPGPPG |
| | COL1A2 | RDGEDGPPGPPGPPGPPGLGGNFAAQYDGAKAGDYGSGPMGLMGPRGPPGT |
| | (protein) | SGPPGPPGFQGPHGEPGEPGQTGPQGPRGPSGPPGKAGEDGHPGKSGRSGERG |
| | | VSGPQGARGFPGTPGLPGFKGIRGHNGLDGQKGQPGTPGIKGESGAPGENGTP |
| | | GQSGARGLPGERGRIGAPGPAGARGSDGSTGPTGPAGPIGSAGAPGFPGAPGA |
| | | KGEIGAAGNVGPSGPAGPRGEAGLPGSSGPVGPPGNPGSNGLAGAKGATGLP |
| | | GVAGAPGLPGPRGIPGPSGPAGAAGTRGLVGEPGPAGAKGESGNKGEPGAAG |
| | | PSGPAGPSGEEGKKGTTGEPGSSGPPGPAGLRGVPGSRGLPGADGRAGVMGP |
| | | AGSRGATGPAGAKGPSGDNGRPGEPGLMGPRGLPGQPGSSGPAGKEGPVGFP |
| | | GADGRVGPTGPAGARGEPGNIGFPGPKGPTGDPGKPGDRGHAGLAGARGAP |
| | | GPEGNNGAQGPPGVAGNPGAKGEQGPAGPPGFQGLPGPSGPAGEAGKPGERG |
| | | MAGEFGAPGPAGSRGERGPPGESGAVGPVGPIGSRGPSGPPGTDGNKGEPGN |
| | | VGNAGTAGPSGAGGAPGERGIAGIPGPKGEKGATGLRGDTGATGRDGARGAP |
| | | GAIGAPGPAGGAGERGEGGPAGAAGPSGARGIPGERGEPGPAGPTGFAGPAG |
| | | AAGQPGAKGERGTKGPKGENGPQGAVGPVGSSGPSGPVGASGPAGPRGDGG |
| | | PPGVTGFPGAAGRTGPPGPSG1TGPPGPPGSAGKDGMRGPRGDTGPVGRTGEQ |
| | | GIVGPPGFSGEKGPSGEPGAAGPPGTPGPQGILGAPGILGLPGSRGERGLPGISG |
| | | ATGEPGPLGISGPPGARGPSGPVGSAGLNGAPGEAGRDGNPGHDGAPGRDGA |
| | | PGFKGERGAPGNNGPAGAVGAPGAHGQVGPAGKPGNRGDPGPVGPSGPAGA |
| | | FGARGPSGPQGARGEKGETGEKGHRGMPGFKGHNGLQGLPGLAGQHGDQGP |
| | | PGSTGPAGPRGPSGPSGPAGKDGRNGLPGPIGPAGVRGSQGSQGPSGPPGPPGL |
| | | PGPPGANGGGYEVGYDLEYYRADQPALRPKDYEVDATLKTLNNQIETLLTPE |
| | | GSRKNPARTCRDIRLSHPEWTSGFYWIDPNQGCTMDAIRVYCDFSTGETCIHA |
| | | NLENIPTKNWYVSKNSKEKKHMWFGETTNGGTQFEYNDEGVTSKDMATQLA |
| | | FMRLLANHASQNITYHCKNSIAYMDEETGNLKKAVILQGSNDVELRAEGNSR |
| | | FTFSVLEDGCSRKNNAWGKTIIEYRTNKPSRLPILDIAPLDIGGADQEFGLDIGP |
| | | VCFK |
| 23 | COLsyn1a | GGTCCTAAGGGTCCAAAGGGCCCTAAGGGACCCAAAGGTCCACCTGGCCC |
| | (DNA) | TCCAGGCGATCCAGGTGACCCTGGCGACCCCGGAGATCCA |
| 24 | COLsyn1a | GPKGPKGPKGPKGPPGPPGDPGDPGDPGDP |
| | (protein) | |
| 25 | COLsyn2 | GCATCGTCTCATCGGTCTCATTCTGGTCCTAAAGGACCCGACGGACCAAAG |
| | (DNA) | GGCCCAGACGGACCCCCTGGTCCACCAGGTGACCCCGGCAAGCCAGGAGA |
| | | TCCCGGTAAACCAATCCTGAGACCTGAGACGGCAT |
| 26 | COLsyn2 | GPKGPDGPKGPDGPPGPPGDPGKPGDPGKP |
| | (protein) | |
| 27 | COLsyn3 | GGACCAAAGGGACCCAAAGGACCAGACGGCCCAGATGGCCCCCCAGGAC |
| | (DNA) | CTCCTGGCGACCCAGGTGACCCAGGTAAGCCTGGCAAGCCT |
| 28 | COLsyn3 | GPKGPKGPDGPDGPPGPPGDPGDPGKPGKP |
| | (protein) | |
| 29 | COLsyn4 | GGTCCTAAAGGACCAAAGGGTCCCAAGGGCCCAAAGGGTCCTCCAGGAGC |
| | (DNA) | TCCTGGACCACCTGGCCCTCCAGGTGTCCCAGGTCCACCA |
| 30 | COLsyn4 | GPKGPKGPKGPKGPPGAPGPPGPPGVPGPP |
| | (protein) | |
| 31 | COLsyn5 | GGTCCTGACGGACCTGATGGACCAGATGGTCCTGATGGTCCTCCAGGAGC |
| | (DNA) | TCCTGGACCACCTGGCCCTCCAGGTGTCCCAGGTCCACCA |
| 32 | COLsyn5 | GPDGPDGPDGPDGPPGAPGPPGPPGVPGPP |
| | (protein) | |
| 33 | COLsyn6 | GGTTTAGCTGGTCCCCCAGGTCCTGCAGGAGCTCCCGGTCCTCCAGGAGCT |
| | (DNA) | CCTGGACCACCTGGCCCTCCAGGTGTCCCAGGTCCACCA |
| 34 | COLsyn6 | GLAGPPGPAGAPGPPGAPGPPGPPGVPGPP |
| | (protein) | |
| 35 | GFP- | ATGCGTAAAGGCGAAGAGCTGTTCACTGGTGTCGTCCCTATTCTGGTGGAA |
| | COLsyn2- | CTGGATGGTGATGTCAACGGTCATAAGTTTTCCGTGCGTGGCGAGGGTGA |
| | ePTS1 | AGGTGACGCAACTAATGGTAAACTGACGCTGAAGTTCATCTGTACTACTG |
| | (DNA) | GTAAACTGCCGGTTCCTTGGCCGACTCTGGTAACGACGCTGACTTATGGTG |
| | | TTCAGTGCTTTGCTCGTTATCCGGACCATATGAAGCAGCATGACTTCTTCA |
| | | AGTCCGCCATGCCGGAAGGCTATGTGCAGGAACGCACGATTTCCTTTAAG |
| | | GATGACGGCACGTACAAAACGCGTGCGGAAGTGAAATTTGAAGGCGATAC |
| | | CCTGGTAAACCGCATTGAGCTGAAAGGCATTGACTTTAAAGAGGACGGCA |
| | | ATATCCTGGGCCATAAGCTGGAATACAATTTTAACAGCCACAATGTTTACA |
| | | TCACCGCCGATAAACAAAAAAATGGCATTAAAGCGAATTTTAAAATTCGC |
| | | CACAACGTGGAGGATGGCAGCGTGCAGCTGGCTGATCACTACCAGCAAAA |
| | | CACTCCAATCGGTGATGGTCCTGTTCTGCTGCCAGACAATCACTATCTGAG |
| | | CACGCAAAGCGTTCTGTCTAAAGATCCGAACGAGAAACGCGATCATATGG |
| | | TTCTGCTGGAGTTCGTAACCGCAGCGGGCATCACGCATGGTATGGATGAA |
| | | CTGTACAAAGCATCGTCTCATCGGTCTCATTCTGGTCCTAAAGGACCCGAC |
| | | GGACCAAAGGGCCCAGACGGACCCCCTGGTCCACCAGGTGACCCCGGCAA |
| | | GCCAGGAGATCCCGGTAAACCAATCCTGAGACCTGAGACGGCATTTGGGA |
| | | AGAGGTAGAAGATCCAAATTG |
| 36 | GFP- | MRKGEELFTGVVPILVELDGDVNGHKFSVRGEGEGDATNGKLTLKFICTTGK |
| | COLsyn2- | LPVPWPTLVTTLTYGVQCFARYPDHMKQHDFFKSAMPEGYVQERTISFKDDG |
| | ePTS1 | TYKTRAEVKFEGDTLVNRIELKGIDFKEDGNILGHKLEYNFNSHNVYITADKQ |
| | (protein) | KNGIKANFKIRHNVEDGSVQLADHYQQNTPIGDGPVLLPDNHYLSTQSVLSK |
| | | DPNEKRDHMVLLEFVTAAGITHGMDELYKGPKGPDGPKGPDGPPGPPGDPGK |
| | | PGDPGKPLGRGRRSKL |
| 37 | GFP- | ATGCGTAAAGGCGAAGAGGTGTTCACTGGTGTCGTCCCTATTCTGGTGGAA |
| | COLsyn3- | CTGGATGGTGATGTCAACGGTCATAAGTTTTCCGTGCGTGGCGAGGGTGA |
| | ePTS1 | AGGTGACGCAACTAATGGTAAACTGACGCTGAAGTTCATCTGTACTACTG |
| | (DNA) | GTAAACTGCCGGTTCCTTGGCCGACTCTGGTAACGACGCTGACTTATGGTG |
| | | TTCAGTGCTTTGCTCGTTATCCGGACCATATGAAGCAGCATGACTTCTTCA |
| | | AGTCCGCCATGCCGGAAGGCTATGTGCAGGAACGCACGATTTCCTTTAAG |
| | | GATGACGGCACGTACAAAACGCGTGCGGAAGTGAAATTTGAAGGCGATAC |
| | | CCTGGTAAACCGCATTGAGCTGAAAGGCATTGACTTTAAAGAGGACGGCA |
| | | ATATCCTGGGCCATAAGCTGGAATACAATTTTAACAGCCACAATGTTTACA |
| | | TCACCGCCGATAAACAAAAAAATGGCATTAAAGCGAATTTTAAAATTCGC |
| | | CACAACGTGGAGGATGGCAGCGTGCAGCTGGCTGATCACTACCAGCAAAA |
| | | CACTCCAATCGGTGATGGTCCTGTTCTGCTGCCAGACAATCACTATCTGAG |
| | | CACGCAAAGCGTTCTGTCTAAAGATCCGAACGAGAAACGCGATCATATGG |
| | | TTCTGCTGGAGTTCGTAACCGCAGCGGGCATCACGCATGGTATGGATGAA |
| | | CTGTACAAAGGACCAAAGGGACCCAAAGGACCAGACGGCCCAGATGGCC |
| | | CCCCAGGACCTCCTGGCGACCCAGGTGACCCAGGTAAGCCTGGCAAGCCT |
| | | TTGGGAAGAGGTAGAAGATCCAAATTG |
| 38 | GFP- | MRKGEELFTGVVPILVELDGDVNGHKFSVRGEGEGDATNGKLTLKFICTTGK |
| | COLsyn3- | LPVPWPTLVTTLTYGVQCFARYPDHMKQHDFFKSAMPEGYVQERTISFKDDG |
| | ePTS1 | TYKTRAEVKFEGDTLVNRIELKGTDFKEDGNILGHKLEYNFNSHNVYTTADKQ |
| | (protein) | KNGIKANFKIRHNVEDGSVQLADHYQQNTPIGDGPVLLPDNHYLSTQSVLSK |
| | | DPNEKRDHMVLLEFVTAAGITHGMDELYKGPKGPKGPDGPDGPPGPPGDPGD |
| | | PGKPGKPLGRGRRSKL |
| 39 | GFP- | ATGCGTAAAGGCGAAGAGCTGTTCACTGGTGTCGTCCCTATTCTGGTGGAA |
| | COLsyn6- | CTGGATGGTGATGTCAACGGTCATAAGTTTTCCGTGCGTGGCGAGGGTGA |
| | ePTS1 | AGGTGACGCAACTAATGGTAAACTGACGCTGAAGTTCATCTGTACTACTG |
| | (DNA) | GTAAACTGCCGGTTCCTTGGCCGACTCTGGTAACGACGCTGACTTATGGTG |
| | | TTCAGTGCTTTGCTCGTTATCCGGACCATATGAAGCAGCATGACTTCTTCA |
| | | AGTCCGCCATGCCGGAAGGCTATGTGCAGGAACGCACGATTTCCTTTAAG |
| | | GATGACGGCACGTACAAAACGCGTGCGGAAGTGAAATTTGAAGGCGATAC |
| | | CCTGGTAAACCGCATTGAGCTGAAAGGCATTGACTTTAAAGAGGACGGCA |
| | | ATATCCTGGGCCATAAGCTGGAATACAATTTTAACAGCCACAATGTTTACA |
| | | TCACCGCCGATAAACAAAAAAATGGCATTAAAGCGAATTTTAAATTCGC |
| | | CACAACGTGGAGGATGGCAGCGTGCAGCTGGCTGATCACTACCAGCAAAA |
| | | CACTCCAATCGGTGATGGTCCTGTTCTGCTGCCAGACAATCACTATCTGAG |
| | | CACGCAAAGCGTTCTGTCTAAAGATCCGAACGAGAAACGCGATCATATGG |
| | | TTCTGCTGGAGTTCGTAACCGCAGCGGGCATCACGCATGGTATGGATGAA |
| | | CTGTACAAAGGTTTAGCTGGTCCCCCAGGTCCTGCAGGAGCTCCCGGTCCT |
| | | CCAGGAGCTCCTGGACCACCTGGCCCTCCAGGTGTCCCAGGTCCACCATTG |
| | | GGAAGAGGTAGAAGATCCAAATTG |
| 40 | GFP- | MRKGEELFTGVVPILVELDGDVNGHKFSVRGEGEGDATNGKLTLKFICTTGK |
| | COLsyn6- | LPVPWPTLVTTLTYGVQCFARYPDHMKQHDFFKSAMPEGYVQERTISFKDDG |
| | ePTS1 | TYKTRAEVKFEGDTLVNRIELKGIDFKEDGNILGHKLEYNFNSHNVYITADKQ |
| | (protein) | KNGIKANFKIRHNVEDGSVQLADHYQQNTPIGDGPVLLPDNHYLSTQSVLSK |
| | | DPNEKRDHMVLLEFVTAAGITHGMDELYKGLAGPPGPAGAPGPPGAPGPPGP |
| | | PGVPGPPLGRGRRSKL |
| 41 | Btau | ATGATCTGGTATATTTTAGTTGTAGGGATTCTACTTCCCCAGTCTTTGGCCC |
| | P4HA1 | ATCCAGGCTTTTTTACTTCTATTGGTCAGATGACTGATTTGATTCATACTGA |
| | (DNA) | AAAAGATCTGGTGACTTCCCTGAAAGACTATATAAAGGCAGAAGAGGACA |
| | | AATTAGAACAAATAAAAAAATGGGCAGAGAAATTAGATCGATTAACCAGC |
| | | ACAGCGACAAAAGATCCAGAAGGATTTGTTGGACACCCTGTAAATGCATT |
| | | CAAATTAATGAAACGTCTGAACACTGAGTGGAGTGAGTTGGAGAATCTGG |
| | | TCCTTAAGGATATGTCAGATGGTTTTATCTCTAACCTAACCATTCAGAGAC |
| | | AGTACTTCCCTAATGATGAAGATCAGGTTGGGGCAGCCAAAGCTCTGTTGC |
| | | GTCTACAGGACACCTACAATTTGGATACAGATACCATCTCAAAGGGTGAT |
| | | CTTCCAGGAGTAAAACACAAATCTTTTCTAACAGTTGAGGACTGTTTTGAG |
| | | TTGGGCAAAGTGGCCTACACAGAAGCAGATTATTACCATACAGAGCTGTG |
| | | GATGGAACAAGCACTGAGGCAGCTGGATGAAGGCGAGGTTTCTACCGTTG |
| | | ATAAAGTCTCTGTTCTGGATTATTTGAGCTATGCAGTATACCAGCAGGGAG |
| | | ACCTGGATAAGGCGCTTTTGCTCACAAAGAAGCTTCTTGAACTAGATCCTG |
| | | AACATCAGAGAGCTAACGGTAACTTAAAATACTTTGAGTATATAATGGCT |
| | | AAAGAAAAAGATGCCAATAAGTCTTCTTCAGATGACCAATCTGATCAGAA |
| | | AACCACACTGAAGAAGAAAGGTGCTGCTGTGGATTACCTGCCAGAGAGAC |
| | | AGAAGTACGAAATGCTGTGCCGTGGGGAGGGTATCAAAATGACTCCTCGG |
| | | AGACAGAAAAAACTCTTCTGTCGCTACCATGATGGAAACCGGAATCCTAA |
| | | ATTTATCCTGGCTCCAGCCAAACAGGAGGATGAGTGGGACAAGCCTCGTA |
| | | TTATCCGCTTCCATGATATTATTTCTGATGCAGAAATTGAAGTCGTTAAAG |
| | | ATCTAGCAAAACCAAGGCTGAGGCGAGCCACCATTTCAAACCCAATAACA |
| | | GGAGACTTGGAGACGGTACATTACAGAATTAGCAAAAGTGCCTGGCTGTC |
| | | TGGCTATGAAAACCCTGTGGTGTCACGAATTAATATGAGAATCCAAGATCT |
| | | GACAGGACTAGATGTCTCCACAGCAGAGGAATTACAGGTAGCAAATTATG |
| | | GAGTTGGAGGACAGTATGAACCCCATTTTGATTTTGCACGGAAAGATGAG |
| | | CCAGATGCTTTCAAAGAGCTGGGGACAGGAAATAGAATTGCTACATGGCT |
| | | GTTTTATATGAGTGATGTGTTAGCAGGAGGAGCCACTGTTTTTCCTGAAGT |
| | | AGGAGCTAGTGTTTGGCCCAAAAAGGGAACTGCTGTTTTCTGGTATAATCT |
| | | GTTTGCCAGTGGAGAAGGAGATTATAGTACACGGCATGCAGCCTGTCCAG |
| | | TGCTGGTTGGAAACAAATGGGTATCCAATAAATGGCTCCATGAACGTGGA |
| | | CAGGAATTTCGAAGACCATGCACCTTGTCAGAATTGGAATGA |
| 42 | Btau | MIWYILVVGILLPQSLAHPGFFTSIGQMTDLIHTEKDLVTSLKDYIKAEEDKLE |
| | P4HA1 | QIKKWAEKLDRLTSTATKDPEGFVGHPVNAFKLMKRLNTEWSELENLVLKD |
| | (protein) | MSDGFISNLTIQRQYFPNDEDQVGAAKALLRLQDTYNLDTDTISKGDLPGVKH |
| | | KSFLTVEDCFELGKVAYTEADYYHTELWMEQALRQLDEGEVSTVDKVSVLD |
| | | YLSYAVYQQGDLDKALLLTKKLLELDPEHQRANGNLKYFEYIMAKEKDANK |
| | | SSSDDQSDQKTTLKKKGAAVDYLPERQKYEMLCRGEGIKMTPRRQKKLFCR |
| | | YHDGNRNPKFILAPAKQEDEWDKPRIIRFHDIISDAEIEVVKDLAKPRLRRATIS |
| | | NPITGDLETVHYRISKSAWLSGYENPVVSRINMRIQDLTGLDVSTAEELQVAN |
| | | YGVGGQYEPHFDFARKDEPDAFKELGTGNRIATWLFYMSDVLAGGATVFPE |
| | | VGASVWPKKGTAVFWYNLFASGEGDYSTRHAACPVLVGNKWVSNKWLHER |
| | | GQEFRRPCTLSELE |
| 43 | BtauP4HB | ATGCTGCGCCGCGCTCTGCTCTGCCTGGCCCTGACCGCGCTATTCCGCGCG |
| | (DNA) | GGTGCCGGCGCCCCCGACGAGGAGGACCACGTCCTGGTGCTCCATAAGGG |
| | | CAACTTCGACGAGGCGCTGGCGGCCCACAAGTACCTGCTGGTGGAGTTCT |
| | | ACGCCCCATGGTGCGGCCACTGCAAGGCTCTGGCCCCGGAGTATGCCAAA |
| | | GCAGCTGGGAAGCTGAAGGCAGAAGGTTCTGAGATCAGACTGGCCAAGGT |
| | | GGATGCCACTGAAGAGTCTGACCTGGCCCAGCAGTATGGTGTCCGAGGCT |
| | | ACCCCACCATCAAGTTCTTCAAGAATGGAGACACAGCTTCCCCCAAAGAG |
| | | TACACAGCTGGCCGAGAAGCGGATGATATCGTGAACTGGCTGAAGAAGCG |
| | | CACGGGCCCCGCTGCCAGCACGCTGTCCGACGGGGCTGCTGCAGAGGCCT |
| | | TGGTGGAGTCCAGTGAGGTGGCCGTCATTGGCTTCTTCAAGGACATGGAGT |
| | | CGGACTCCGCAAAGCAGTTCTTCTTGGCAGCAGAGGTCATTGATGACATCC |
| | | CCTTCGGGATCACATCTAACAGCGATGTGTTCTCCAAATACCAGCTGGACA |
| | | AGGATGGGGTTGTCCTCTTAAGAAGTTTGACGAAGGCCGGAACAACTTT |
| | | GAGGGGGAGGTCACCAAAGAAAAGCTTCTGGACTTCATCAAGCACAACCA |
| | | GTTGCCCCTGGTCATTGAGTTCACCGAGCAGACAGCCCCGAAGATCTTCGG |
| | | AGGGGAAATCAAGACTCACATCCTGCTGTTCCTGCCGAAAAGCGTGTCTG |
| | | ACTATGAGGGCAAGCTGAGCAACTTCAAAAAAGCGGCTGAGAGCTTCAAG |
| | | GGCAAGATCCTGTTTATCTTCATCGACAGCGACCACACTGACAACCAGCGC |
| | | ATCCTGGAATTCTTCGGCCTAAAGAAAGAGGAGTGCCCGGCCGTGCGCCT |
| | | CATCACGCTGGAGGAGGAGATGACCAAATATAAGCCAGAGTCAGATGAGC |
| | | TGACGGCAGAGAAGATCACCGAGTTCTGCCACCGCTTCCTGGAGGGCAAG |
| | | ATTAAGCCCCACCTGATGAGCCAGGAGCTGCCTGACGACTGGGACAAGCA |
| | | GCCTGTCAAAGTGCTGGTTGGGAAGAACTTTGAAGAGGTTGCTTTTGATGA |
| | | GAAAAAGAACGTCTTTGTAGAGTTCTATGCCCCGTGGTGCGGTCACTGCAA |
| | | GCAGCTGGCCCCCATCTGGGATAAGCTGGGAGAGACGTACAAGGACCACG |
| | | AGAACATAGTCATCGCCAAGATGGACTCCACGGCCAACGAGGTGGAGGCG |
| | | GTGAAAGTGCACAGCTTCCCCACGCTCAAGTTCTTCCCCGCCAGCGCCGAC |
| | | AGGACGGTCATCGACTACAATGGGGAGCGGACACTGGATGGTTTTAAGAA |
| | | GTTCCTGGAGAGTGGTGGCCAGGATGGGGCCGGAGATGATGACGATCTAG |
| | | AAGATCTTGAAGAAGCAGAAGAGCCTGATCTGGAGGAAGATGATGATCAA |
| | | AAAGCTGTGAAAGATGAACTGTAA |
| 44 | BtauP4HB | MLRRALLCLALTALFRAGAGAPDEEDHVLVLHKGNFDEALAAHKYLLVEFY |
| | (protein) | APWCGHCKALAPEYAKAAGKLKAEGSEIRLAKVDATEESDLAQQYGVRGYP |
| | | TIKFFKNGDTASPKEYTAGREADDIVNWLKKRTGPAASTLSDGAAAEALVESS |
| | | EVAVIGFFKDMESDSAKQFFLAAEVIDDIPFGITSNSDVFSKYQLDKDGVVLFK |
| | | KFDEGRNNFEGEVTKEKLLDFIKHNQLPLVIEFTEQTAPKIFGGEIKTHILLFLP |
| | | KSVSDYEGKLSNFKKAAESFKGKILFIFIDSDHTDNQRILEFFGLKKEECPAVRL |
| | | ITLEEEMTKYKPESDELTAEKITEFCHRFLEGKIKPHLMSQELPDDWDKQPVK |
| | | VLVGKNFEEVAFDEKKNVFVEFYAPWCGHCKQLAPIWDKLGETYKDHENIVI |
| | | AKMDSTANEVEAVKVHSFPTLKFFPASADRTVIDYNGERTLDGFKKFLESGG |
| | | QDGAGDDDDLEDLEEAEEPDLEEDDDQKAVKDEL |
| 45 | BtP4HB | GCCCCCGACGAGGAGGACCACGTCCTGGTGCTCCATAAGGGCAACTTCGA |
| | (DNA) | CGAGGCGCTGGCGGCCCACAAGTACCTGCTGGTGGAGTTCTACGCCCCAT |
| | | GGTGCGGCCACTGCAAGGCTCTGGCCCCGGAGTATGCCAAAGCAGCTGGG |
| | | AAGCTGAAGGCAGAAGGTTCTGAGATCAGACTGGCCAAGGTGGATGCCAC |
| | | TGAAGAGTCTGACCTGGCCCAGCAGTATGGTGTCCGAGGCTACCCCACCA |
| | | TCAAGTTCTTCAAGAATGGAGACACAGCTTCCCCCAAAGAGTACACAGCT |
| | | GGCCGAGAAGCGGATGATATCGTGAACTGGCTGAAGAAGCGCACGGGCCC |
| | | CGCTGCCAGCACGCTGTCCGACGGGGCTGCTGCAGAGGCCTTGGTGGAGT |
| | | CCAGTGAGGTGGCCGTCATTGGCTTCTTCAAGGACATGGAGTCGGACTCCG |
| | | CAAAGCAGTTCTTCTTGGCAGCAGAGGTCATTGATGACATCCCCTTCGGGA |
| | | TCACATCTAACAGCGATGTGTTCTCCAAATACCAGCTGGACAAGGATGGG |
| | | GTTGTCCTCTTTAAGAAGTTTGACGAAGGCCGGAACAACTTTGAGGGGGA |
| | | GGTCACCAAAGAAAAGCTTCTGGACTTCATCAAGCACAACCAGTTGCCCC |
| | | TGGTCATTGAGTTCACCGAGCAGACAGCCCCGAAGATCTTCGGAGGGGAA |
| | | ATCAAGACTCACATCCTGGTGTTCCTGCCGAAAAGCGTGTCTGACTATGAG |
| | | GGCAAGCTGAGCAACTTCAAAAAAGCGGCTGAGAGCTTCAAGGGCAAGAT |
| | | CCTGTTTATCTTCATCGACAGCGACCACACTGACAACCAGCGCATCCTGGA |
| | | ATTCTTCGGCCTAAAGAAAGAGGAGTGCCCGGCCGTGCGCCTCATCACGC |
| | | TGGAGGAGGAGATGACCAAATATAAGCCAGAGTCAGATGAGCTGACGGC |
| | | AGAGAAGATCACCGAGTTCTGCCACCGCTTCCTGGAGGGCAAGATTAAGC |
| | | CCCACCTGATGAGCCAGGAGCTGCCTGACGACTGGGACAAGCAGCCTGTC |
| | | AAAGTGCTGGTTGGGAAGAACTTTGAAGAGGTTGCTTTTGATGAGAAAAA |
| | | GAACGTCTTTGTAGAGTTCTATGCCCCGTGGTGCGGTCACTGCAAGCAGCT |
| | | GGCCCCCATCTGGGATAAGCTGGGAGAGACGTACAAGGACCACGAGAAC |
| | | ATAGTCATCGCCAAGATGGACTCCACGGCCAACGAGGTGGAGGCGGTGAA |
| | | AGTGCACAGCTTCCCCACGCTCAAGTTCTTCCCCGCCAGCGCCGACAGGAC |
| | | GGTCATCGACTACAATGGGGAGCGGACACTGGATGGTTTTAAGAAGTTCC |
| | | TGGAGAGTGGTGGCCAGGATGGGGCCGGAGATGATGACGATCTAGAAGAT |
| | | CTTGAAGAAGCAGAAGAGCCTGATCTGGAGGAAGATGATGATCAAAAAG |
| | | CTGTGAAAGATGAACTG |
| 46 | BtP4HB | APDEEDHVLVLHKGNFDEALAAHKYLLVEFYAPWCGHCKALAPEYAKAAG |
| | (protein) | KLKAEGSEIRLAKVDATEESDLAQQYGVRGYPTIKFFKNGDTASPKEYTAGRE |
| | | ADDIVNWLKKRTGPAASTLSDGAAAEALVESSEVAVIGFFKDMESDSAKQFF |
| | | LAAEVIDDIPFGITSNSDVFSKYQLDKDGVVLFKKFDEGRNNFEGEVTKEKLL |
| | | DFIKHNQLPLVIEFTEQTAPKIFGGEIKTHILLFLPKSVSDYEGKLSNFKKAAESF |
| | | KGKILFIFIDSDHTDNQRILEFFGLKKEECPAVRLITLEEEMTKYKPESDELTAE |
| | | KITEFCHRFLEGKIKPHLMSQELPDDWDKQPVKVLVGKNFEEVAFDEKKNVF |
| | | VEFYAPWCGHCKQLAPIWDKLGETYKDHENIVIAKMDSTANEVEAVKVHSFP |
| | | TLKFFPASADRTVIDYNGERTLDGFKKFLESGGQDGAGDDDDLEDLEEAEEPD |
| | | LEEDDDQKAVKDEL |
| 47 | GFP- | ATGCGTAAAGGCGAAGAGCTGTTCACTGGTGTCGTCCCTATTCTGGTGGAA |
| | BtP4HB- | CTGGATGGTGATGTCAACGGTCATAAGTTTTCCGTGCGTGGCGAGGGTGA |
| | ePTSl | AGGTGACGCAACTAATGGTAAACTGACGCTGAAGTTCATCTGTACTACTG |
| | (DNA) | GTAAACTGCCGGTTCCTGGCCGACTCTGGTAACGACGCTGACTTATGGTG |
| | | TTCAGTGCTTTGCTCGTTATCCGGACCATATGAAGCAGCATGACTTCTTCA |
| | | AGTCCGCCATGCCGGAAGGCTATGTGCAGGAACGCACGATTTCCTTTAAG |
| | | GATGACGGCACGTACAAAACGCGTGCGGAAGTGAAATTTGAAGGCGATAC |
| | | CCTGGTAAACCGCATTGAGCTGAAAGGCATTGACTTTAAAGAGGACGGCA |
| | | ATATCCTGGGCCATAAGCTGGAATACAATTTTAACAGCCACAATGTTTACA |
| | | TCACCGCCGATAAACAAAAAAATGGCATTAAAGCGAATTTTAAAATTCGC |
| | | CACAACGTGGAGGATGGCAGCGTGCAGCTGGCTGATCACTACCAGCAAAA |
| | | CACTCCAATCGGTGATGGTCCTGTTCTGCTGCCAGACAATCACTATCTGAG |
| | | CACGCAAAGCGTTCTGTCTAAAGATCCGAACGAGAAACGCGATCATATGG |
| | | TTCTGCTGGAGTTCGTAACCGCAGCGGGCATCACGCATGGTATGGATGAA |
| | | CTGTACAAAGCCCCCGACGAGGAGGACCACGTCCTGGTGCTCCATAAGGG |
| | | CAACTTCGACGAGGCGCTGGCGGCCCACAAGTACCTGCTGGTGGAGTTCT |
| | | ACGCCCCATGGTGCGGCCACTGCAAGGCTCTGGCCCCGGAGTATGCCAAA |
| | | GCAGCTGGGAAGCTGAAGGCAGAAGGTTCTGAGATCAGACTGGCCAAGGT |
| | | GGATGCCACTGAAGAGTCTGACCTGGCCCAGCAGTATGGTGTCCGAGGCT |
| | | ACCCCACCATCAAGTTCTTCAAGAATGGAGACACAGCTTCCCCCAAAGAG |
| | | TACACAGCTGGCCGAGAAGCGGATGATATCGTGAACTGGCTGAAGAAGCG |
| | | CACGGGCCCCGCTGCCAGCACGCTGTCCGACGGGGCTGCTGCAGAGGCCT |
| | | TGGTGGAGTCCAGTGAGGTGGCCGTCATTGGCTTCTTCAAGGACATGGAGT |
| | | CGGACTCCGCAAAGCAGTTCTTCTTGGCAGCAGAGGTCATTGATGACATCC |
| | | CCTTCGGGATCACATCTAACAGCGATGTGTTCTCCAAATACCAGCTGGACA |
| | | AGGATGGGGTTGTCCTCTTTAAGAAGTTTGACGAAGGCCGGAACAACTTT |
| | | GAGGGGGAGGTCACCAAAGAAAAGCTTCTGGACTTCATCAAGCACAACCA |
| | | GTTGCCCCTGGTCATTGAGTTCACCGAGCAGACAGCCCCGAAGATCTTCGG |
| | | AGGGGAAATCAAGACTCACATCCTGCTGTTCCTGCCGAAAAGCGTGTCTG |
| | | ACTATGAGGGCAAGCTGAGCAACTTCAAAAAAGCGGCTGAGAGCTTCAAG |
| | | GGCAAGATCCTGTTTATCTTCATCGACAGCGACCACACTGACAACCAGCGC |
| | | ATCCTGGAATTCTTCGGCCTAAAGAAAGAGGAGTGCCCGGCCGTGCGCCT |
| | | CATCACGCTGGAGGAGGAGATGACCAAATATAAGCCAGAGTCAGATGAGC |
| | | TGACGGCAGAGAAGATCACCGAGTTCTGCCACCGCTTCCTGGAGGGCAAG |
| | | ATTAAGCCCCACCTGATGAGCCAGGAGCTGCCTGACGACTGGGACAAGCA |
| | | GCCTGTCAAAGTGCTGGTTGGGAAGAACTTTGAAGAGGTTGCTTTTGATGA |
| | | GAAAAAGAACGTCTTTGTAGAGTTCTATGCCCCGTGGTGCGGTCACTGCAA |
| | | GCAGCTGGCCCCCATCTGGGATAAGCTGGGAGAGACGTACAAGGACCACG |
| | | AGAACATAGTCATCGCCAAGATGGACTCCACGGCCAACGAGGTGGAGGCG |
| | | GTGAAAGTGCACAGCTTCCCCACGCTCAAGTTCTTCCCCGCCAGCGCCGAC |
| | | AGGACGGTCATCGACTACAATGGGGAGCGGACACTGGATGGTTTTAAGAA |
| | | GTTCCTGGAGAGTGGTGGCCAGGATGGGGCCGGAGATGATGACGATCTAG |
| | | AAGATCTTGAAGAAGCAGAAGAGCCTGATCTGGAGGAAGATGATGATCAA |
| | | AAAGCTGTGAAAGATGAACTGTGGGAAGAGGTAGAAGATCCAAATTG |
| 48 | GFP- | MRKGEELFTGVVPILVELDGDVNGHKFSVRGEGEGDATNGKLTLKFICTTGK |
| | BtP4HB- | LPVPWPTLVTTLTYGVQCFARYPDHMKQHDFFKSAMPEGYVQERTISFKDDG |
| | ePTS1 | TYKTRAEVKFEGDTLVNRIELKGIDFKEDGNILGHKLEYNFNSHNVYITADKQ |
| | (protein) | KNGIKANFKIRHNVEDGSVQLADHYQQNTPIGDGPVLLPDNHYLSTQSVLSK |
| | | DPNEKRDHMVLLEFVTAAGITHGMDELYKAPDEEDHVLVLHKGNFDEALAA |
| | | HKYLLVEFYAPWCGHCKALAPEYAKAAGKLKAEGSEIRLAKVDATEESDLA |
| | | QQYGVRGYPTIKFFKNGDTASPKEYTAGREADDIVNWLKKRTGPAASTLSDG |
| | | AAAEALVESSEVAVIGFFKDMESDSAKQFFLAAEVTDDIPFGITSNSDVFSKYQ |
| | | LDKDGVVLFKKFDEGRNNFEGEVTKEKLLDFIKHNQLPLVIEFTEQTAPKIFGG |
| | | EIKTHILLFLPKSVSDYEGKLSNFKKAAESFKGKILFIFIDSDHTDNQRILEFFGL |
| | | KKEECPAVRLITLEEEMTKYKPESDELTAEKITEFCHRFLEGKIKPHLMSQELP |
| | | DDWDKQPVKVLVGKNFEEVAFDEKKNVFVEFYAPWCGHCKQLAPIWDKLG |
| | | ETYKDHENIVIAKMDSTANEVEAVKVHSFPTLKFFPASADRTVIDYNGERTLD |
| | | GFKKFLESGGQDGAGDDDDLEDLEEAEEPDLEEDDDQKAVKDELLGRGRRS |
| | | KL |
| 49 | TEV | GGAGAGTCCCTGTTTAAAGGACCCAGAGACTATAACCCGATTAGTAGCAC |
| | protease | TATTTGTCATCTTACAAACGAAAGTGATGGTCACACGACTAGTCTTTACGG |
| | (DNA) | AATCGGATTCGGCCCATTTATTATCACAAACAAGCATCTGTTCAGAAGAAA |
| | | TAACGGGACGTTGTTGGTCCAATCTCTTCATGGAGTATTTAAGGTAAAGAA |
| | | CACTACAACTCTTCAGCAGCATCTGATCGACGGTAGGGATATGATCATCAT |
| | | CCGTATGCCGAAAGACTTTCCACCTTTTCCTCAGAAGTTGAAGTTTAGAGA |
| | | ACCCCAGCGTGAGGAGCGTATCTGTTTAGTAACAACAAATTTCCAAACGA |
| | | AATCTATGTCATCAATGGTTAGCGATACCAGTTGTACTTTCCCCAGTTCAG |
| | | ATGGGATTTTCTGGAAGCACTGGATTCAGACAAAGGACGGTCAGTGTGGT |
| | | AGTCCGCTTGTTTCTACAAGGGACGGATTTATTGTCGGGATACACAGTGCT |
| | | TCTAACTTTACGAATACAAACAACTACTTCACGTCTGTCCCTAAAAATTTT |
| | | ATGGAGCTGTTGACTAATCAGGAAGCCCAACAGTGGGTATCTGGCTGGCG |
| | | TTTGAACGCGGATTCCGTACTGTGGGGTGGCCACAAGGTTTTTATGGTTAA |
| | | GCCTGAAGAGCCGTTCCAACCTGTGAAGGAGGCAACACAGCTAATGAAT |
| 50 | TEV | GESLFKGPRDYNPISSTICHLTNESDGHTTSLYGIGFGPFIITNKHLFRRNNGTLL |
| | protease | VQSLHGVFKVKNTTTLQQHLIDGRDMIIIRMPKDFPPFPQKLKFREPQREERIC |
| | (protein) | LVTTNFQTKSMSSMVSDTSCTFPSSDGIFWKHWIQTKDGQCGSPLVSTRDGFI |
| | | VGIHSASNFTNTNNYFTSVPKNFMELLTNQEAQQWVSGWRLNADSVLWGGH |
| | | KVFMVKPEEPFQPVKEATQLMN |
| 69 | T4 | GGTTACATCCCCGAAGCTCCTCGTGACGGCCAGGCTTACGTCAGGAAAGA |
| | fibritin | TGGCGAGTGGGTTCTTTTGTCCACTTTTCTG |
| | foldon | |
| | domain | |
| | (DNA) | |
| 70 | T4 | GYIPEAPRDGQAYVRKDGEWVLLSTFL |
| | fibritin | |
| | foldon | |
| | domain | |
| | (protein) | |

TABLE 2
Details for Sequences of Table 1

| SEQ ID | | |
|---|---|---|
| NO: | Type | Details |
| 15 + 16 | cargo to peroxisome, | NM_001034039, Bos taurus |
| | substrate for modification | collagen type I alpha |
| | | 1 chain (COL1A1) |
| 17 + 18 | cargo to peroxisome, | NM_174520, Bos taurus |
| | substrate for modification | collagen type I alpha |
| | | 2 chain (COL1A2) |
| 19 + 20 | cargo to peroxisome, | XM_006277058, PREDICTED: |
| | substrate for modification | Alligator mississippiensis |
| | | collagen type I alpha |
| | | 1 chain (COL1A1) |
| 21 + 22 | cargo to peroxisome, | XM_006258452, PREDICTED: |
| | substrate for modification | Alligator mississippiensis |
| | | collagen type I alpha |
| | | 2 chain (COL1A2), |
| | | transcript variant X1 |
| 23 + 24 | cargo to peroxisome, | synthetic collagen peptide |
| | substrate for modification | |
| 25 + 26 | cargo to peroxisome, | synthetic collagen peptide |
| | substrate for modification | |
| 27 + 28 | cargo to peroxisome, | synthetic collagen peptide |
| | substrate for modification | |
| 29 + 30 | cargo to peroxisome, | synthetic collagen peptide |
| | substrate for modification | |
| 31 + 32 | cargo to peroxisome, | synthetic collagen peptide |
| | substrate for modification | |
| 33 + 34 | cargo to peroxisome, | synthetic collagen peptide |
| | substrate for modification | |
| 35 + 36 | cargo to peroxisome, | fusion protein, GFP for |
| | substrate for modification | Western and fluorescence, |
| | | ePTS1 for peroxisome |
| | | localization |
| 37 + 38 | cargo to peroxisome, | fusion protein, GFP for |
| | substrate for modification | Western and fluorescence, |
| | | ePTS1 for peroxisome |
| | | localization |
| 39 + 40 | cargo to peroxisome, | fusion protein, GFP for |
| | substrate for modification | Western and fluorescence, |
| | | ePTS1 for peroxisome |
| | | localization |
| 41 + 42 | cargo to peroxisome, | NM_001075770, Bos taurus |
| | modification enzyme | prolyl 4-hydroxylase subunit |
| | (hydroxylation) | alpha 1 (P4HA1) |
| 43 + 44 | cargo to peroxisome, | NM_174135, Bos taurus |
| | modification enzyme | prolyl 4-hydroxylase subunit |
| | (hydroxylation, protein | beta (P4HB) |
| | disulfide isomerization) | |
| 45 + 46 | cargo to peroxisome, | lacks N-term SS |
| | substrate for modification | |
| 47 + 48 | cargo to peroxisome, | fusion protein, GFP for |
| | substrate for modification | Western and fluorescence, |
| | | ePTS1 for peroxisome |
| | | localization |
| 49 + 50 | modifying enzyme, | |
| | protease | |

### Example 2: Protection from Toxic Compound

In some embodiments, targeting a protein and/or enzyme to a peroxisome compartmentalizes it by physically separating from another enzyme or substrate. This may be used to prevent interaction or activity between the separated protein(s), enzyme(s), and/or substrate(s). For example, a toxic or inhibitory protein such as SigD may be compartmentalized.

Peroxisome compartmentalization of an enzyme to physically separate it from its substrate is used in some embodiments to prevent activity on the substrate. To illustrate the ability to compartmentalize activity, cell viability is rescued when a toxic protein is expressed by sequestering the toxic protein in the peroxisome.

The pathogen bacteria Salmonella is a common cause of gastroenteritis by invading the intestinal mucosa. One of the pathogenic factors secreted by Salmonella is SigD, a putative inositol phosphatase that has been demonstrated to cause severe growth inhibition when expressed in S. cerevisiae. The toxicity is linked to the SigD N-terminal domain (SigD1-351) that lacks the phosphatase domain but affects the organization of the actin cytoskeleton in both yeast and human cells (doi:10.1111/j.1462-5822.2005.00568.x).

By removing access of SigD1-351 to its cytoplasmic actin cytoskeleton substrate by peroxisome compartmentalization, S. cerevisiae can be protected from SigD inhibitory growth effects.

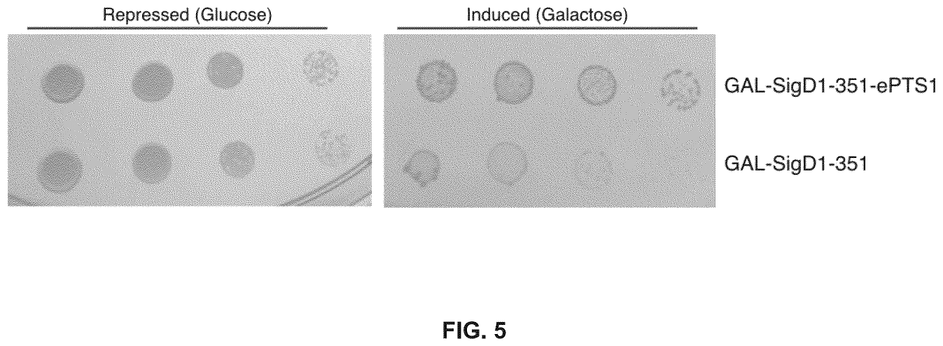

FIG. 5 is an example to demonstrate the protection conferred to the host S. cerevisiae when the toxic protein SigD1-351 is sequestered in the peroxisome. The strains, integrated with either SigD1-351-eTPS1 or SigD1-351 under the control of the inducible GAL promoter, were serially diluted on YPD plates to repress expression or YPGalactose place to induce expression. When repressed, both strains grew equally well. When expression was induced, the strain with the peroxisome localized toxin (SigD1-351-eTPS1) was able to grow but the cytoplasmically expressed toxin (SigD1-351) was lethal to the host.

An example includes the following design: use of expression cassettes with an inducible GAL promoter to control toxic SigD expression, expression of a toxic (SigD1-351) and non-toxic variant (SigD1-351(118-142A)) of SigD in separate expression cassettes transformed into yeast cells, production of fusion proteins GFP-x-ePTS1 by the expression cassettes, where x is a toxic or a non-toxic SigD variant, and transformation of separate groups of yeast cells each with one of the following strain backgrounds: PEX5 (peroxisome import) and pex5Δ (lacks peroxisome import. In this example, the following laboratory techniques are performed: serial dilutions of cells on glucose (repressed) and galactose (induced) plates to show growth defects, and demonstration of localization by GFP fluorescence.

### Example 3: Co-Localization of Enzyme and Substrate to Perform Post-Translational Modification in Peroxisome

Various classes of post-translational modifications (PTMs) can be demonstrated to occur in peroxisomes. Separation of an enzyme and its substrate or protein substrate by peroxisome barrier is used to prevent activity of the enzyme on the substrate in some embodiments. Thus, sequestration of a substrate or enzyme can be used. For example, this may be an example of protection of cellular content from peroxisome-sequestered protein or vice versa.

In some embodiments, a modification enzyme that performs a post-translational modification (PTM) on another protein is co-localized with the other protein in the peroxisome of a cell. Examples of PTMs include but are not limited to glycosylation (or other sugar additions), isomerization, cleavage, protease cleavage, proteolytic degradation, hydroxylation, proteolysis, phosphorylation, dephosphorylation, ubiquitination (and ubiquitin-like modifications like neddylation, sumoylation), methylation, nitrosylation, acetylation, and lipidation (including GPI anchoring, prenylation, myristolation). Other PTM reactions are also contemplated. In some embodiments, an enzyme, any of the enzyme's co-factors, and the enzyme's substrate are co-localized to the cytoplasm and/or peroxisome.

In some embodiments, an enzyme, any of the enzyme's co-factors, and the enzyme's substrate are co-localized to the cytoplasm and/or peroxisome. This is used in some embodiments to demonstrate that when the enzyme and substrate are co-localized in the same region, the modification occurs. Thus, co-localization may be used to perform a modification such as a PTM.

Examples of PTMs suitable for use in the methods and compositions disclosed herein include protease cleavage, phosphorylation, dephosphorylation, hydroxylation, isomerization, glycosylation, and prenylation. In some embodiments, one or more of protease cleavage, phosphorylation and dephosphorylation are preferred PTMs.

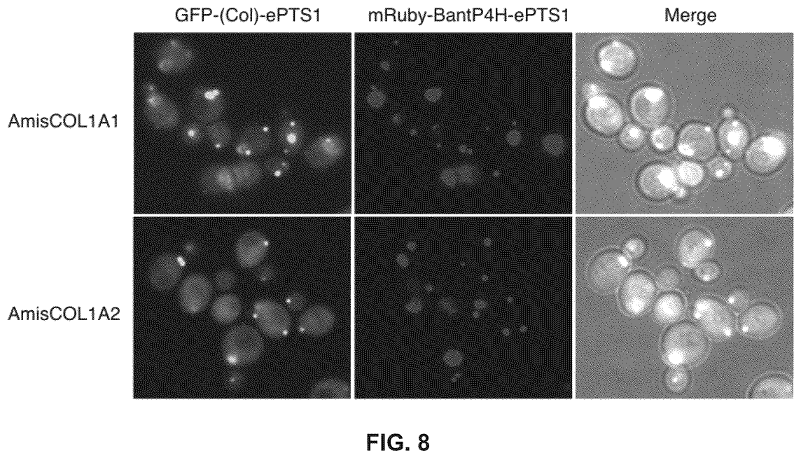

FIG. 8 demonstrates the in vivo co-localization of a hydroxylase enzyme (BantP4H) and a collagen substrate (AmisCOL1A1 or Amis COL1A2) in the S. cerevisiae. BantP4H contains a mRuby fusion tag and the collagen substrate with GFP fusion tag to monitor localization by fluorescence microscopy. Fluorescent foci are observed with the ePTS1 peroxisome localization signal and the merged images demonstrate the overlapping localization of the hydroxylase and collagen. Exemplary sequences having mRuby may include, for example, SEQ ID NOs: 51-52.

### Example 4: Proteolysis

In some embodiments, TEV protease is used to demonstrate that peptide cleavage can occur in the peroxisome. For example, in some embodiments, cleavage can only occurs when both the protease and substrate are in the same subcellular compartment (such as the cytoplasm or peroxisome). The example demonstrating the TEV protease is sequestered in the peroxisome and cannot cleave its target in the cytoplasm shows that other potential targets in the cytoplasm are also not subject to TEV-cleavage and are thus protected from the peroxisome compartmentalized enzyme. In some embodiments, if an expressed protein/enzyme is toxic to the cell, then separating it from its cellular substrate by peroxisome compartmentalization provides protection to the cell from the protein/enzyme. The example that the substrate/protein is sequestered in the peroxisome and cannot be cleaved by the TEV protease in the cytoplasm suggests that the substrate will also not be subject to other enzymes in the cytoplasm, and thus the substrate/protein is protected from unwanted modifications from the cell such as proteolytic degradation. Thus, in some embodiments, selective targeting of some proteins and not others results in desired modifications of some proteins and/or prevents unwanted modifications.

In some embodiments, in S. cerevisiae, the TEV protease and a substrate containing the TEV recognition site (TEVrs) for cleavage are to be expressed from strong promoters. Fusions to YFP or RFP will demonstrate localization to cytoplasm or peroxisome by microscopy. Proteolysis of substrate (YFP-TEVrs-IGF2-FLAG) will be analyzed by Western blot.

In some embodiments, other modifying proteases that can be targeted to the peroxisome include but not limited to matrix metalloproteinases MMP-1. MMP-2, MMP-8, MMP-13, and MMP-14; N-proteinases ADAMTS-2, ADAMTS-3, ADAMTS-14; and C-proteinases BMP-1, mTLS, and TLL-1.

In some embodiments, proteins targeted to the peroxisome contain a TEV-cleavable tag. By way of example, an example of a protein with a cleavable tag is BtCol1A2-TEV-GFP-HIS-ePTS1 (SEQ ID NO: 64), where the full-length bovine collagen type1 alpha 2 protein can be separated by TEV protease from an N-terminal tag that can be used for peroxisome localization, visualization, and purification. Additional examples can include any protein sequence as disclosed herein in combination with any tag sequence, targeting sequence, domain, or fragment, or derivative thereof. Examples of such sequences can include, for example SEQ ID NOs: 57-68.

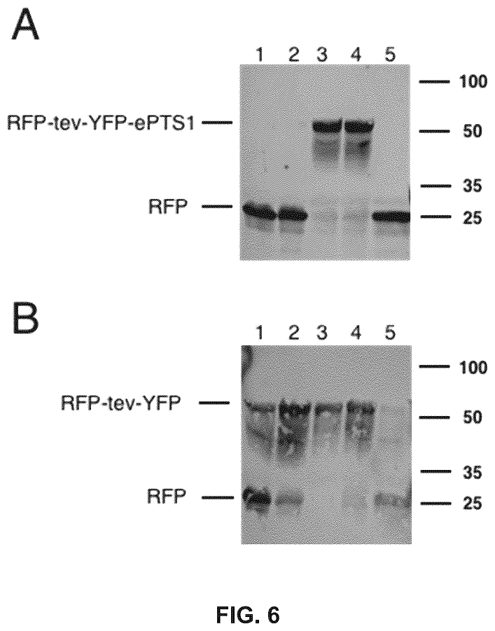

The TEV protease is a sequence specific cysteine protease from the Tobacco Etch Virus (TEV). In this example to demonstrate heterologous enzyme activity could be achieved in the peroxisome, the TEV protease was expressed in S. cerevisiae with an N-terminal ePTS1 signal sequence to direct its localization to the peroxisome. The substrate created to test for TEV activity was created by flanking the TEV recognition amino acid sequence, Glu-Asn-Leu-Tyr-Phe-Gln-Ser, by an N-terminal RFP and C-terminal YFP. This substrate was expressed either with (FIG. 6, panel A) or without the ePTS1 sequence (FIG. 6, panel B). When the TEV protease and substrate were both expressed and co-localized in the peroxisome, the substrate was completely cleaved as evidenced by the disappearance of the 54 kDa full-sized substrate band and appearance of the 27 kDa RFP cleavage product on the Western blot (FIG. 6, panel A, lanes 1, 2, and 5). However, when the expression of TEV protease was repressed, the peroxisome-localized substrate remained uncut (FIG. 6, panel A, lanes 3 and 4). As a control, the substrate was expressed in the cytoplasm but TEV protease targeted to the peroxisome. Varying amounts of substrate cleavage were observed and were directly correlated to the strength of the promoter driving TEV protease expression, pRPL18B<pTEF1<pGAL1 (FIG. 6, panel B, lanes 1, 2, and 5). These results suggest that TEV protease was still active in the cytoplasm as it was being imported into the peroxisome but was dependent on high expression to access the substrate. Comparatively, TEV cleavage activity was complete when the substrate and protease were co-localized in the peroxisome despite differences in expression levels of the TEV protease demonstrating an example of how co-compartmentalization can also improve the efficiency of substrate modification.

### Example 5: Phosphorylation and Dephosphorylation

In some embodiments, a specific kinase (such as a serine/threonine kinase or a tyrosine kinase) and/or a phosphatase and their substrates are identified to co-express. For example, MEK and its substrate MAPK1 may be encoded in a nucleic acid or in separate nucleic acids to produce fusion peptides of MEK and MAPK1 with peroxisome-targeting peptides to target the MEK and MAPK1 to the peroxisome where MEK phosphorylates MAPK1. Additionally, further enzymes and substrates may be added, for example, Raf-1.

### Example 6: Hydroxylation

In some embodiments, collagen hydroxylation in a peroxisome by a P4H dioxygenase is demonstrated. For example, a design with bovine P4H subunits may be used. Alternatively, a single bacterial P4H (Bacillus anthracis or mimivirus) may be used. In some embodiments, media is supplemented with ascorbic acid and/or α-ketoglutarate and iron(II), and it is demonstrated that if co-factors and/or supplements and can enter the peroxisome then specific chemical modifications can occur there. In such a case, collagen is analyzed for oxidation by mass-spectroscopy. In some embodiments, an in vitro assay is used to further demonstrate enzyme activity.

To demonstrate heterologous hydroxylation activity could be achieved in the peroxisome in vivo, a prolyl-4-hydroxylase (P4H) enzyme and a collagen substrate were co-expressed in S. cerevisiae. The P4H enzyme from Bacillus anthracis has previously been demonstrated to hydroxylate synthetic collagen-like peptides in vitro (Schnicker and Dey, 2016) and was expressed either in the cytoplasm (BantP4H) or the peroxisome (BantP4H-ePTS1). The collagen helix is composed of GXY repeats, where G is glycine, X is any amino acid but often proline, and Y is any amino acid but often proline. Prolines in the Y position are preferentially hydroxylated for helical stability (Gorres and Raines, 2010). The substrate designed for this study was a 99 amino acid fragment of the helical region of bovine collagen type 1 alpha 1 that contains 11 Y-position prolines (BtCol1A1 403-11P). To control for Y-position proline hydroxylation, the 11 prolines were mutated to alanine or valine (BtCol1A1 403-OP). These substrates were expressed with an N-terminal GFP to monitor in vivo localization (see FIG. 8) and for purification as well as a C-terminal ePTS1 peroxisome-localization sequence.

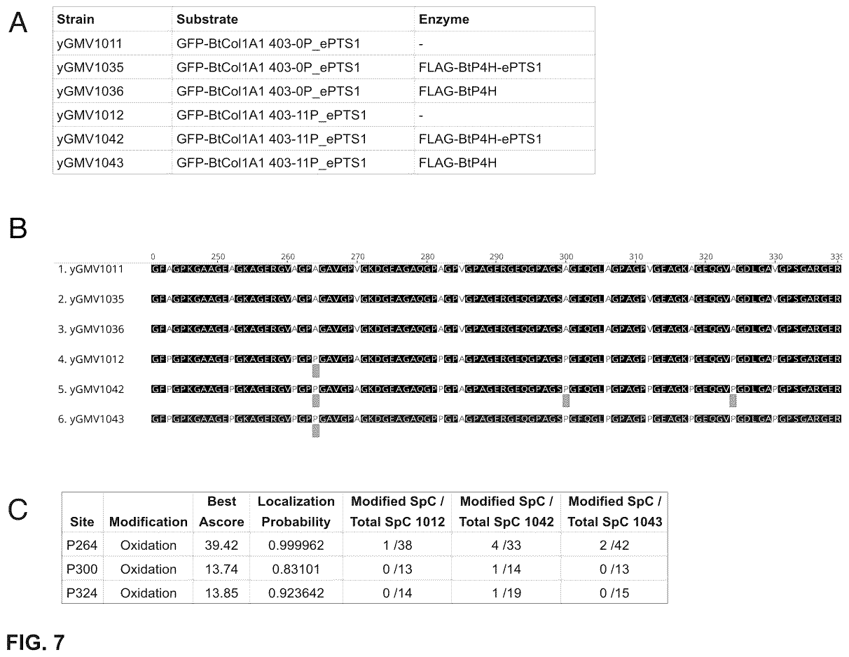

Cells expressing a combination of the BantP4H enzyme and collagen substrate (FIG. 7, panel A) were grown in YPD in baffled shake flasks at 30 C to early log phase and then harvested. Following cell lysis, the substrates were purified on GFP-Trap beads, run on a 10% PAGE gel, stained with Coomassie Blue, excised from the gel, and sent to MS Bioworks for analysis by LCMSMS for oxidation of proline residues.

Mass spectroscopy results revealed BantP4H-specific oxidation at three sites on the collagen substrate when co-expressed in the peroxisome. The BtCol1A1 403-11P_ePTS1 substrate was oxidized in on position P264, a Y-position proline, in strains PB000225, PB000254, and PB000255. The corresponding position in the BtCol1A1 403-0P_ePTS1 control substrate was mutated to alanine (A264) and no oxidation was observed (FIG. 7, panel B). Upon closer inspection of the modifications identified at P264, there is 12.1% oxidation at this position in strain PB000254 (four modified/33 total) in which the BantP4H is co-localized in the peroxisome compared to 2.6% and 4.8% in strains PB000225 (one modified/38 total) and PB000225 (two modified/42 total), respectively. Similarly, oxidation at two additional Y-position prolines, P300 and P324, was only observed in strain PB000254 and not in the other five strains (FIG. 7, panel C). Together, these results show three Y-position prolines on the collagen substrate to be specifically hydroxylated by the Bant-P4H when both enzyme and substrate are co-localized to the peroxisome. Exemplary sequences having a 403-OP-ePTS1 or 403-11P-ePTS1 include, for example, SEQ ID NOs: 53-56 and 65-68.

### Example 7: Expression of Collagen in Yeast Peroxisome

Collagen protein is imported into the peroxisome via a peroxisome targeting tag. A prolyl hydroxylase and prolyl isomerase are similarly imported into the peroxisome using a peroxisome targeting tag. Co-incubation of the prolyl hydroxylase enzyme with collagen in the peroxisome allows the formation of the proper triple helix conformation. Type I heterotrimer, Type 1 alpha homotrimer, and Type III homotrimer collagen are all produced in the manner described. For collagen type I, both full-length Col1A1 (pro-alpha1 chain) and Col1A2 (pro-alpha2 chain) are expressed as well as truncations of both the N- and C-termini to isolate the teloprotein shown by Olsen et al (2001) for improved expression of Col1A1 (alpha1 chain) and Col1A2 (alpha2 chain) in S. cerevisiae. Similarly, prolyl-4-hydroxylase is expressed as full-length as well as a truncation of the PDI domain (Toman 2000) for improved expression and import into the peroxisome.

### Example 8: Increasing Cargo of the Peroxisome

Yeast is grown in a fermenter using any of a variety of conventional protocols. Peroxisome capacity can be increased through induction. In the case of S. cerevisiae this may be through the use of oleate and for Pichia pastoris and Ogataea polymorpha this may be through the use of methanol. Proteins desired to be compartmentalized and purified are tagged with a peroxisome-targeting tag: PTS1, PTS2, or enhanced versions of these tags. Post-fermentation, the plasma membranes of the yeast cells can be lysed using many conventional lysing methods such as French press or cell wall digestion using a lyticase followed by homogenization. Low-speed centrifugation is used to remove nuclei and plasma membrane and other cellular debris. The peroxisomes may be further purified from the resultant supernatant by other methods such as a density gradient centrifugation. An alternative method of peroxisome purification is to genetically tag a peroxisome membrane protein with an affinity tag such as streptavidin or a polyhistidine peptide to allow affinity purification. These purified peroxisomes are then lysed; for example, using an osmotic lysis (J Cell Biol. 2007 Apr. 23; 177(2): 289-303; included by reference in its entirety herein). The peroxisome debris can be removed via a high-speed centrifugation and the soluble fraction containing the desired cargo protein collected. If desired, this desired protein can be further purified using an affinity purification. Without being limiting, cargo proteins may be tagged with any of a number of available peptide or protein fold affinity tags such as, for example, a poly-histidine, maltose-binding protein, glutathione S-transferase, and purified using their respective protocols. Alternatively, other purification methods such as ion chromatography or gel filtration may be used.

### Example 9: Expression of Post-Translationally Modified Proteins in Yeast Peroxisome—Localization of Individual Proteins to Peroxisome (ePTS1-Based Targeting)

Different classes of proteins based on size and function are demonstrated to localize to peroxisomes in a typical yeast cell through the use a peroxisome targeting sequence. Non-limiting examples of proteins and types of proteins that can be targeted are listed in Table 3. The mechanism of peroxisome targeting is conserved, and therefore the platform can be used in other organisms including methylotrophic yeasts such as Pichia pastoris/Komagataella phaffii, Hansenula polymorpha/Ogataea parapolymorpha, and Candida boidinii. GFP-x-ePTS 1 and x-FLAG-ePTS1 constructs are produced. In the constructs, GFP is used for visualization of localization, FLAG-ePTS1 for protein expression and in case GFP interferes with function), and “x” represents the protein or enzyme of interest to be targeted. Some construct sequences and details of some embodiments are provided in Tables 1 and 2.

TABLE 3

| Protein (x) | Function | Size (kDa) |
|---|---|---|
| TEV | Modifying enzyme- protease | 52 |
| RFP-TEV | Modifying enzyme- protease RFO | 78 |
| | fusion to demonstrate localization | |
| IGF-II | Protein hormone similar to insulin | 20.7 |
| YFP-TEVrs-IFGII | Protease substrate | 27 |
| GFP | | 26 |
| Tyrosine kinase | Modifying enzyme- phosphorylation | |
| Tyrosine kinase | Kinase/phosphatase substrate | |
| substrate | | |
| Tyrosine | Modifying enzyme- | |
| phosphatase | dephosphorylation | |
| BtauP4HA1 | Modifying enzyme- hydroxylase | 59 |
| BtauP4HB | Modifying enzyme- isomerase | 55 |
| Collagen peptides | | 5 |

### Example 10: Disulfide Bond Formation

In some embodiments, the modification is a disulfide bond formation. For example, a design wherein a heterologous protein and a protein disulfide isomerase (PDI) are co-expressed and targeted to the peroxisome is used. In such a case, the heterologous protein is analyzed for disulfides by mass-spectroscopy.

To demonstrate disulfide bond formation in the peroxisome in vivo, heterologous genes expressing human insulin, alpha interferon, and mapacalcine are co-expressed along with a PDI. An Ogataea PDI (OgPDI) that is usually targeted to the ER is designed to be overexpressed and targeted to the peroxisome. Human insulin precursor (Baeshan et al, 2014), alpha interferon (Shi et al, 2007) and mapacalcine (Noubhani et al, 2015) are synthesized using optimized codons from Pichia pastoris. The constructs are designed with three expression cassettes, including an expression cassette for the target gene of interest, an expression cassette for the modifying enzyme, and an expression cassette for the selectable marker.

Each cassette has a promoter, the expressed gene (gene of interest or modifying enzyme gene or selectable marker gene) and a terminator. The gene of interest and the modifying enzyme genes are designed to include fluorescent tags GFP and mRuby, respectively, as translational fusions. Both the gene of interest and the modifying enzyme are targeted to the peroxisome by the introduction of the ePTS1 sequence at the 3′ end. The sequence of the entire construct co-expressing mapacalcine and OgPDI is set forth in SEQ ID NO: 73. Additional cassettes include an nucleic acid sequence for human insulin precursor (SEQ ID NO: 74), alpha interferon (SEQ ID NO: 75), mapacalcine (SEQ ID NO: 76), OgPDI (SEQ ID NO: 77)

The transgenics expressing these cassettes are screened initially for the fluorescence markers confirming targeting to the peroxisomes. The heterologous proteins of interest purified from the transgenic strains are analyzed for disulfide formation by mass spectrometry.

### Example 11: Phosphorylation

In some embodiments, the modification is a phosphorylation. For example, human beta-casein II (Greenberg et al, 1984; Thurmond et al, 1997) and a specific protein kinase, namely human casein kinase (Voss et al, 1991) that phosphorylates specific serine and threonine amino acids on the casein are identified for co-expression. Codon optimized sequences of the human beta-casein II is set forth in SEQ ID NO: 78 and of the casein kinase II subunit beta is set forth in SEQ ID NO: 79.

The constructs for transformation are generated using the same backbone used for the demonstration of the disulfide bond formation (as set forth in Example 10). Casein is used as the gene of interest and casein kinase is used as the modifying enzyme. Phosphorylation is a major form of regulation in the peroxisome, and the target casein expressed in the peroxisome may not even require the co-expression of the casein kinase. Once generated, the recombinant casein is purified and analyzed for phosphorylated forms of threonine and serine by mass-spectroscopy. In some embodiments, phosphorylation activity is assayed in vitro.

### Example 12: Acetylation

In some embodiments, the modification is N-terminal acetylation. For example, hen egg ovalbumin (Ito & Matsudomi, 2005) and a specific acetylation complex NatB (Rovere et al, 2008) that facilitates acetylation of N-terminal glycine are identified for co-expression. Codon optimized sequences of the ovalbumin is set forth in SEQ ID NO: 80 and two genes corresponding to the yeast NatB complex (Naa20 and Naa25) are set forth in SEQ ID NOs: 81 and 82, respectively.

The constructs for transformation are generated using the same backbone used for the demonstration of the disulfide bond formation (as described in Example 10). Ovalbumin is used as the gene of interest and the two genes of the NatB complex constitute the modifying enzyme. Many proteins in yeasts are acetylated at the N-terminus, and the target ovalbumin expressed in the peroxisome may show N-terminal acetylation even in the absence of the casein kinase. Once generated the recombinant casein is purified and analyzed for acetylation of the N-terminal glycine by mass-spectroscopy.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of one aspect is applicable to all aspects and embodiments identified herein. Moreover, any of the features of an embodiment of one aspect is independently combinable, partly or wholly with other embodiments described herein in any way, e.g., one, two, or three or more embodiments may be combinable in whole or in part. Further, any of the features of an embodiment of one aspect may be made optional to other aspects or embodiments.

1. (canceled)

2. A method of producing a modified protein in a peroxisome of a eukaryotic cell, the method comprising:
culturing a eukaryotic cell comprising a peroxisome;
expressing in the eukaryotic cell a heterologous protein comprising a peroxisome-targeting sequence, whereby the heterologous protein is localized to the peroxisome;
expressing in the eukaryotic cell a heterologous modification enzyme comprising a peroxisome-targeting sequence, whereby the heterologous modification enzyme is localized to the peroxisome; and
modifying the heterologous protein in the peroxisome with the heterologous modification enzyme, thereby producing the modified protein that comprises a modification.

3. The method of claim 2, wherein the eukaryotic cell is a yeast cell.

4. The method of claim 3, wherein the yeast cell is a genus of Arxula, Candida, Hansenula, Kluyveromyces, Komagataella, Ogataea, Pichia, Saccharomyces, or Yarrowia.

5. The method of claim 2, wherein the peroxisome-targeting sequence comprises a sequence as set forth in SEQ ID NO: 1, SEQ ID NO: 2, or SEQ ID NO: 3.

6. The method of claim 2, wherein the peroxisome-targeting sequence comprises an enhanced PTS1 sequence comprising LGRGRRSKL.

7. The method of claim 2, wherein the heterologous modification enzyme is a prolyl hydroxylase, prolyl-4-hydroxylase, lysyl oxidase, prolyl isomerase, protein disulfide isomerase, chaperone protein, hydroxyl transferase, oxidase, protease, kinase, phosphatase, or glycosyltransferase.

8. The method of claim 2, wherein the modification comprises hydroxylation, oxidation, proteolysis, phosphorylation, dephosphorylation, isomerization, disulfide bond formation or rearrangement, folding, assembly, or glycosylation.

9. The method of claim 2, further comprising expressing in the eukaryotic cell a second heterologous modification enzyme comprising a peroxisome-targeting sequence, whereby the second heterologous modification enzyme is localized to the peroxisome.

10. The method of claim 2, wherein the heterologous protein is collagen, gelatin, silk protein, a collagen fragment, a collagen peptide, a collagen-like peptide, or a synthetic collagen-like protein.

11. The method of claim 2, wherein the heterologous protein comprises collagen or a collagen-like protein, wherein the heterologous modification enzyme comprises a prolyl hydroxylase, and wherein the modification comprises hydroxylation of one or more proline residues in the collagen or collagen-like protein.

12. The method of claim 11, wherein the prolyl hydroxylase comprises prolyl-4-hydroxylase.

13. The method of claim 11, wherein the collagen or collagen-like protein comprises COL1A1, COL1A2, COLsyn2, COLsyn3, or an amino acid sequence at least 80%, 85%, 90%, 95%, 97%, 98%, or 99% identical to COLsyn2 or COLsyn3, wherein COLsyn2 and COLsyn3 comprise the amino acid sequences set forth in SEQ ID NO: 26 and SEQ ID NO: 28, respectively.

14. The method of claim 11, wherein the modified protein comprises a Type I collagen heterotrimer, a Type I collagen alpha homotrimer, a Type III collagen homotrimer, or a collagen chain modified by hydroxylation at one or more Y-position proline residues.

15. The method of claim 2, wherein localization of the heterologous protein and the heterologous modification enzyme to the peroxisome increases yield of the modified protein relative to a corresponding cell in which the heterologous protein or the heterologous modification enzyme is not localized to the peroxisome.

16. The method of claim 15, further comprising increasing cargo capacity of the peroxisome.

17. The method of claim 16, wherein increasing cargo capacity of the peroxisome comprises providing oleic acid or methanol to the eukaryotic cell.

18. An engineered eukaryotic cell comprising a peroxisome and comprising:
a first nucleic acid encoding a heterologous protein comprising a peroxisome-targeting sequence; and
a second nucleic acid encoding a heterologous modification enzyme comprising a peroxisome-targeting sequence;
wherein the engineered eukaryotic cell is configured to express the heterologous protein and the heterologous modification enzyme such that the heterologous protein and the heterologous modification enzyme are localized to the peroxisome and the heterologous modification enzyme modifies the heterologous protein in the peroxisome.

19. The engineered eukaryotic cell of claim 18, wherein the engineered eukaryotic cell is a yeast cell of a genus of Arxula, Candida, Hansenula, Kluyveromyces, Komagataella, Ogataea, Pichia, Saccharomyces, and Yarrowia.

20. The engineered eukaryotic cell of claim 18, wherein:
the heterologous protein comprises collagen, gelatin, silk protein, COL1A1, COL1A2, COLsyn2, COLsyn3, or an amino acid sequence at least 80%, 85%, 90%, 95%, 97%, 98%, or 99% identical to COLsyn2 or COLsyn3; and/or
the heterologous modification enzyme comprises a prolyl hydroxylase, prolyl-4-hydroxylase, protein disulfide isomerase, prolyl isomerase, lysyl oxidase, protein chaperone, hydroxyl transferase, oxidase, protease, kinase, phosphatase, or glycosyltransferase.

21. A modified protein comprising:
a collagen or collagen-like protein modified by hydroxylation at one or more proline residues;
wherein the modified protein is produced by:
culturing a eukaryotic cell comprising a peroxisome;
expressing in the eukaryotic cell a heterologous protein comprising a peroxisome-targeting sequence, whereby the heterologous protein is localized to the peroxisome;
expressing in the eukaryotic cell a heterologous modification enzyme comprising a peroxisome-targeting sequence, whereby the heterologous modification enzyme is localized to the peroxisome; and
modifying the heterologous protein in the peroxisome with the heterologous modification enzyme, thereby producing the modified protein.
